(19) United States
(12) Patent Application Publication
(10) Pub. No.: US 2013/0089560 A1
(43) Pub. Date: Apr. 11, 2013

(54) BONE MORPHOGENETIC PROTEIN RECEPTOR BINDING AGENTS AND METHODS OF THEIR USE

(71) Applicants: Cecile Chartier-Courtaud, Palo Alto, CA (US); Austin L. Gurney, San Francisco, CA (US)

(72) Inventors: Cecile Chartier-Courtaud, Palo Alto, CA (US); Austin L. Gurney, San Francisco, CA (US)

(73) Assignee: OncoMed Pharmaceuticals Inc, Redwood City, CA (US)

(21) Appl. No.: 13/635,116

(22) PCT Filed: Mar. 17, 2011
(86) PCT No.: PCT/US11/28850

§ 371 Date: Dec. 14, 2012

Related U.S. Application Data
(60) Provisional application No. 61/314,894, filed on Mar. 17, 2010.
(60) Provisional application No. 61/359,610, filed on Jun. 29, 2010.

(51) Int. Cl. C07K 16/28; A61K 45/06; C07K 16/46; A61K 39/395

(52) U.S. Cl. CPC C07K 16/2863; A61K 39/39558; A61K 45/06; C07K 16/468
USPC 424/143.1; 530/388.22; 530/387.3; 530/388.15; 536/23.53; 435/375; 530/389.1; 435/334

(57) ABSTRACT

The present invention provides bone morphogenetic protein receptor (BMPR) binding agents, such as antibodies, and compositions comprising said binding agents. The binding agents are useful to treat diseases such as cancer.

### CROSS REFERENCE TO RELATED APPLICATIONS

This application claims the priority benefit of U.S. Provisional Application No. 61/314,894, filed Mar. 17, 2010 and U.S. Provisional Application No. 61/359,610, filed Jun. 29, 2010, each of which is hereby incorporated by reference herein in its entirety.

### FIELD OF THE INVENTION

The present invention provides bone morphogenetic protein receptor (BMPR) binding agents, such as monoclonal antibodies, and compositions comprising said binding agents. Also provided are methods of using the BMPR-binding agents for the treatment of diseases such as cancer.

### BACKGROUND OF THE INVENTION

Cancer is one of the leading causes of death in the developed world, resulting in over 550,000 deaths per year in the United States alone. Almost one and half million people are diagnosed with cancer in the U.S. each year, and currently one in four deaths in the U.S. is due to cancer. (Jemal et al., 2008, Cancer J. Clin. 58:71-96). Although there are many drugs and compounds currently available and in use, these numbers show that a need continues to exist for new therapeutic agents for the treatment of cancer.

Cancers and tumors consist of a heterogeneous population of cells. Emerging evidence has shown that only a small subset of cells, referred to as “cancer stem cells” or “CSCs”, have high tumorigenic capacity. The rest of the cancer cells, called non-tumorigenic cancer cells, have little or no tumorigenic capacity. The cancer stem cells, like normal stem cells, have the capability for self-renewal, while the non-tumorigenic cells which constitute the bulk of a tumor are often more differentiated and do not have this capability. Studies have demonstrated that tumors arising from purified tumorigenic cancer stem cells contain a mixture of both tumorigenic and non-tumorigenic cells, similar to the original tumor. (See, e.g., Al-Hajj et al., 2004, Oncogene 23:7274-7282; Dalerba et al., Annual Rev. Med. 2007, 58:267-284). Recently, cancer stem cells have been isolated from breast cancer, prostate cancer, pancreatic cancer and brain cancer (See, e.g., Al-Hajj et al., 2003, PNAS, 100:3983-3988; Singh et al., 2004, Nature, 432:396-401; Patrawala et al., 2006, Oncogene 25:1696-1708; Li et al., 2007, Cancer Research 67:1030-1037).

It is believed by those of skill in the art that both chemo- and radiation therapies reduce tumor size by eliminating the hulk of the non-tumorigenic cells while mostly sparing the CSCs. This provides a strong basis for the observed recurrence of most cancers, and a clear rationale for the development of therapeutic agents that target the CSC population.

The behavior of CSCs may be caused by the malfunction and/or alteration of a number of signaling pathways involved in normal stem cell biology that underlies embryonic development and adult tissue homeostasis. Among these signaling pathways are the Wnt, Hedgehog, Notch and TGF-β/BMP pathways. There can be interplay and/or cross-talk between these pathways which is usually tightly regulated, both spatially and temporally, and can give rise to very complex signaling interactions.

Bone morphogenetic proteins (BMPs) are extracellular signaling molecules that belong to the transforming growth factor-β (TGF-β) superfamily, which in mammals includes approximately 33 members. Of these TGF-β members, more than a dozen have been classified into the BMP subfamily. The BMPs include BMP2, BMP3, BMP4, BMP5, BMP6, BMP7, BMP8a, BMP8b, BMP9, BMP10 and BMP15. Although BMPs were originally identified as factors involved in the formation of bone and cartilage tissue, they have been shown to demonstrate a wide range of biological effects. BMPs affect structures and processes throughout the entire body, including embryonic patterning and development, tissue homeostasis and regeneration, and stem cell maintenance, function and environment (Varga et al., 2005; Oncogene 24:5713-5721; Wagner, 2007, FEBS J. 274:2968-2976; Miyazono et al., 2010, J. Biochem. 147:35-51). Furthermore, BMPs have been shown to regulate proliferation, differentiation, and apoptosis in many different cell types by modulating the transcription of specific target genes.

At the molecular level, BMPs transduce their signals primarily through a heterotetrameric complex comprising transmembrane type I and type II serine/threonine kinase receptors. In mammals there are three type I receptors and three type II receptors that have been identified for BMPs. They include type I receptors BMPR1A (ALK-3), BMPR1B (ALK-6) and ACVR1A (ALK-2) and type II receptors BMPR2 (BMPR-II), ACVR2A (ACTRII or ACTRIIA) and ACVR2B (ACTRIIB).

Evidence has shown that both type I and type II receptors are required for signal transduction. Upon ligand binding, constitutively active type II receptors phosphorylate type I receptors, triggering activation of the type I receptor and subsequent intracellular SMAD signal transduction cascades. For example, an activated type I receptor phosphorylates intracellular receptor-associated SMADs (SMAD-1, SMAD-5 and/or SMAD-8) which allows SMAD-1, 5, 8 to interact with common partner SMAD4. This complex of SMADs translocates to the nucleus and regulates gene transcription of target genes, including proteins such as p21/Cip1/Waf1, bax, p53, Id1-3, OASIS, Prx2, TIEG, Snail, Hey 1 and Tcf7. (See e.g. reviews Massague, 1998, Annu. Rev. Biochem. 67:753-791; Miyazono et al., 2010, J. Biochem. 147:35-51.)

The complexity of the BMP signaling cascade is partly due to the presence of multiple ligands and multiple receptors, with considerable mixing and matching occurring both at the level of ligand-receptor interactions and type I-type II receptor interactions (Schmierer & Hill, 2007, Nature Rev. Mol. Cell Biol. 8:970-982). In addition, there are a number of molecules in the extracellular space that act as negative regulators of the BMP pathway. Some are BMP-binding proteins that inhibit BMP signaling by sequestering BMPs from their receptors. Examples of such negative regulators include, but are not limited to, Gremlin, Noggin, and Chordin. Regulators of the BMP pathway may also inhibit the activity of BMPs by fostering their retention in the endoplasmic reticulum. Other regulators of the BMP pathway, such as BAMBI, interact with various type I and type II receptors and inhibit signaling by the receptors. (See, e.g., Walsh et al. 2010, TICB 20:244-256; Blish et al. 2008, Mol. Biol. Cell, 19:457-464).

The importance of BMPs and their receptors in cancer biology has emerged from several genetic analyses and a variety of in vitro and in vivo studies. BMPs and the BMP pathway have been implicated in both the promotion and the inhibition of tumorigenesis and/or tumor progression. This dual role appears to be dependent upon the BMP, the BMPR, the cancer type, and/or the stage of the cancer.

For example, in primary human NSCLCs, BMP2 was shown to be over-expressed when compared with normal lung or benign lung tumor tissues. Subcutaneous injections of BMP2 with A549 human epithelial NSCLC cells into nude mice were shown to enhance tumor growth, a finding reversed by administration of BMP antagonists. Furthermore, BMP2 was shown to enhance angiogenesis in an in vivo tumor model. (Langenfeld et al. 2003, Carcinogenesis 24:1445-1454 and Langenfeld et al., 2004, Mol. Cancer Res. 2:141-149). BMP6 expression has beer found to be increased in prostate adenocarcinoma and studies have shown a correlation between elevated BMP6 and osteoblastic metastases.

Diminution of ALK1 receptor gene dosage or systemic treatment with a ALK1-Fc fusion protein retarded tumor growth and progression by inhibition of angiogenesis in a transgenic mouse model of multistep tumorigenesis. The effect was shown to result from a signaling synergy between BMP-9 and TGF-β. (Cunha et al. 2010, J. Exp. Med. 207:85-100). It has been shown that BMP9 acts as a proliferative factor for immortalized ovarian surface epithelial cells and ovarian cancer cell lines, signaling predominantly through an ALK2 pathway. In addition, immunohistochemistry analysis revealed that 25% of epithelial ovarian cancers express BMP-9, whereas normal human ovarian surface epithelial specimens do not (Herrera et al. 2009, Cancer Res. 69:9254-9262).

Several reports highlight a change in the BMP response as the tumors progress from primary to metastatic lesions. BMP-7 is expressed at the highest level in advanced castration-resistant PCa cells and the inhibitory effects of BMP-7 are dependent on the differentiation status of PCa cells and the tumor microenvironment (Morrissey et al. 2010, Neoplasia 12:192-205). Increased levels of serum BMP-2 were detected in locally advanced gastric cancer relative to early localized gastric cancer (Park et al. 2009, Med. Oncol. online). Activation of the BMP pathway could be detected in breast cancer bone metastases in vivo. BMP was shown to promote invasion in the same model (Katsuno et al., 2008 Oncogene 27:6322-6333).

In contrast, BMP4 has demonstrated tumor suppressor characteristics in a number of studies. For example, germline mutations in the SMAD4 gene and/or in the BMPR1A gene have been associated with some cases of juvenile polyposis syndrome and Cowden syndrome (Howe et al., 1998, Science 280:1086-1088; Zhou et al., 2001, Am. J. Hum. Genet. 69:704-711). BMP4 treatment of human cancer cells was shown to abrogate the human cancer cells' ability to form xenograft tumors in immunodeficient mice. The cells were a pluripotent, undifferentiated human cancer cell line. (Nishanian et al., 2004, Cancer Biol. & Therapy 3:667-675.) In addition, BMP4 was demonstrated to inhibit the intracerebral grafting of human adult glioblastoma cells in mice, with concurrent reduction in mortality. Reduction of the tumor-initiating cell pool in the treated glioblastoma was shown to be responsible for the effect (Piccirillo et al., 2006, Nature 444:761-765). In a nude mouse model for breast cancer, treatment with BMP7 was shown to inhibit growth of MDA-231-B cells both within bone and orthotopically (Buijs et al., 2007, Cancer Res. 67:8742-8751).

Although BMPs have been demonstrated to have tumor suppressive capabilities, BMP ligands are poorly suited as therapeutic candidates. For example, BMPs possess avid binding to heparin sulfate glycoproteins which may limit systemic delivery, and appear to have inferior pharmacokinetics (PK). Furthermore, BMPs have been shown to possess promiscuous homo- and heterodimerization, and distinct specificity for distinct type I and type II receptor complexes. To overcome these obstacles, the present invention focuses on the generation of highly selective BMP receptor-binding agents directed against specific BMPRs which have the capability to modulate the BMP signaling pathway.

### BRIEF SUMMARY OF THE INVENTION

The present invention provides binding agents, such as antibodies, that specifically recognize at least one bone morphogenetic protein receptor (BMPR), as well as compositions, such as pharmaceutical compositions, comprising the binding agents. In certain embodiments, the binding agents are novel polypeptides, such as antibodies, fragments of such antibodies, and other polypeptides related to such antibodies. In certain embodiments, the binding agents are antibodies that specifically bind BMPR1A, BMPR1B, BMPR2, ACVR2A and/or ACVR2B. In certain embodiments, the binding agents are bispecific antibodies. In some embodiments, the bispecific antibodies bind two different BMPRs. The invention further provides methods of inhibiting the growth of a tumor by administering the binding agents to a subject with a tumor. The invention farther provides methods of treating cancer by administering the binding agents to a subject in need thereof. In some embodiments, the methods of treating cancer or inhibiting tumor growth comprise targeting cancer stem cells with the binding agents. In certain embodiments, the methods comprise reducing the frequency of cancer stem cells in a tumor, reducing the number of cancer stem cells in a tumor, reducing the tumorigenicity of a tumor, and/or reducing the tumorigenicity of a tumor by reducing the number or frequency of cancer stem cells in the tumor. The invention also provides methods of using the binding agents in the treatment of cancer and/or in the inhibition of the growth of tumors comprising cancer stem cells.

In one aspect, the invention provides a binding agent that specifically binds at least one BMPR. In certain embodiments, the agent binding agent is an antibody that specifically binds an extracellular domain of at least one BMPR. In some embodiments, the antibody binds at least one BMPR selected from the group consisting of BMPR1A, BMPR1B, BMPR2, AVRR2A and ACRV2B. In some embodiments, the antibody modulates BMP pathway activity. In some embodiments, the antibody is an agonist of the BMP pathway. In some embodiments, the antibody stimulates and/or enhances signaling of the BMP pathway. In some embodiment, the antibody stimulates and/or enhances activation of the BMP pathway. In certain embodiments, the antibody binds a BMPR which binds a BMP. The BMP may include, but is not limited to, BMP2, BMP3, BMP4, BMP5, BMP6, BMP7, BMP8a, BMP8b, BMP9, BMP10 and BMP15. In certain embodiments, the antibody binds a BMPR which binds BMP4. In certain embodiments, the antibody binds a BMPR which is activated by BMP4.

In certain embodiments, the antibody is an antagonist of the BMP pathway. In some embodiments, the antibody inhibits or interferes with binding of a BMP to a BMPR.

In certain embodiments, the binding agent is an antibody that specifically binds an extracellular domain of BMPR1A, wherein the antibody comprises a heavy chain CDR1 comprising TGYYMK (SEQ ID NO:14), a heavy chain CDR2 comprising RINPDNGGRTYNQIFKDK (SEQ ID NO:15), and a heavy chain CDR3 comprising RERGQYGNYGGFSD (SEQ ID NO:16). In some embodiments, the antibody comprises: (a) a heavy chain CDR1 comprising TGYYMH (SEQ ID NO:14), or a variant thereof comprising 1, 2, 3, or 4 amino acid substitutions; (b) a heavy chain CDR2 comprising RINPDNGGRTYNQIFKDK (SEQ ID NO:15), or a variant thereof comprising 1, 2, 3, or 4 amino acid substitutions; and (c) a heavy chain CDR3 comprising RERGQYGNYGGFSD (SEQ ID NO:16), or a variant thereof comprising 1, 2, 3, or 4 amino acid substitutions. In certain embodiments, the amino acid substitutions are conservative amino acid substitutions. In certain embodiments, the antibody is a bispecific antibody.

In some embodiments, the binding agent is an antibody that specifically binds an extracellular domain of BMPR1A, wherein the antibody comprises heavy chain variable region having at least about 90%, at least about 95%, 96%, 97%, 98% or 99% sequence identity to SEQ ID NO:13. In some embodiments, the antibody comprises a heavy chain variable region comprising the sequence of SEQ ID NO; 13.

In some embodiments, the BMPR-binding agent is antibody 5M107 and is produced by the hybridoma deposited with ATCC having deposit no. PTA-10720. In some embodiments, the BMPR-binding agent is a humanized form of antibody 5M107.

In another embodiment, the invention provides an isolated antibody that competes with the antibody 5M107, produced by the hybridoma deposited with ATCC having deposit number PTA-10720, for binding to BMPR1A. In some embodiments, the BMPR-binding agent is an antibody that specifically binds the same or an over-lapping BMPR epitope as the epitope to which 5M107 binds.

In another aspect, the invention provides a binding agent (e.g., an antibody) that competes for specific binding to an extracellular domain of a human BMPR with an antibody of the invention. In some embodiments, the binding agent (e.g., an antibody) competes for specific binding to an extracellular domain of a BMPR with an antibody that comprises a heavy chain variable region comprising SEQ ID NO:13. In some embodiments, the binding agent competes for specific binding to an extracellular domain of a BMPR with an antibody in an in vitro competitive binding assay.

In another aspect, the invention provides a binding agent that competes for specific binding to an extracellular domain of a human BMPR with antibody 5M107.

In certain embodiments of each of the aforementioned aspects or embodiments, as well as other aspects and/or embodiments described elsewhere herein, the antibody is a recombinant antibody. In certain embodiments, the antibody is a monoclonal antibody, a chimeric antibody, a humanized antibody, or a human antibody. In some embodiments, the antibody is an antibody fragment. In certain embodiments, the antibody or antibody fragment is monovalent, monospecific, bivalent, bispecific, or multispecific. In certain embodiments, the antibody is conjugated to a cytotoxic moiety. In certain embodiments, the antibody is isolated. In still further embodiments, the antibody is substantially pure.

In some embodiments, the BMPR-binding agent is a bispecific antibody. In certain embodiments, the bispecific antibody binds a type I BMPR and a type II BMPR. In some embodiments, the typed BMPR is BMPR1A or BMPR1B. In some embodiments, the type II BMPR is BMPR2, ACVR2A or ACRV2B. In certain embodiments, the bispecific antibody binds BMPR1A and BMPR2. In certain embodiments, the bispecific antibody binds BMPR1B and BMPR2.

In another aspect, the invention provides a binding agent that specifically binds the extracellular domain of a BMPR, wherein the binding agent comprises a polypeptide. In some embodiments, the polypeptide that binds the extracellular domain of a BMPR comprises a polypeptide having at least about 80% sequence identity to SEQ ID NO:13. In some embodiments, the polypeptide is isolated. In certain embodiments, the polypeptide is substantially pure.

In certain embodiments of each of the aforementioned aspects, as well as other aspects described herein, the BMPR-binding agent or polypeptide is an antibody.

In certain embodiments of each of the aforementioned aspects, as well as other aspects described herein, the BMPR-binding agent or polypeptide or antibody stimulates or increases binding of a BMP to a BMPR. In some embodiments, the BMP is BMP4. In some embodiments, the BMPR is BMPR1A. In some embodiments, the BMPR is BMPR1B. In some embodiments, the BMPR is BMPR2. In some embodiments, the BMPR is ACVR2A. In some embodiments, the BMPR is ACVR2B.

In certain embodiments of each of the aforementioned aspects, as well as other aspects described elsewhere herein, the BMPR-binding agent or antibody that specifically binds and/or modulates the activity of one BMPR further specifically binds and/or modulates the activity of a second BMPR.

In certain embodiments of each of the aforementioned aspects, as well as other aspects described elsewhere herein, the BMPR-binding agent is an agonist of the BMP pathway. In certain embodiments, the BMPR-binding agent or antibody stimulates and/or enhances BMP pathway signaling. In some embodiments, the BMPR-binding agent or antibody stimulates and/or enhances BMP pathway activation.

In certain embodiments of each of the aforementioned aspects, as well as other aspects described elsewhere herein, the BMPR-binding agent is an antagonist of the BMP pathway. In some embodiments, the BMPR-binding agent or antibody inhibits the binding of a BMP to a BMPR. In some embodiments, the BMPR-binding agent or antibody inhibits or blocks BMPR signaling. In some embodiments, the BMPR-binding agent or antibody inhibits or blocks BMPR activation.

In another aspect, the invention provides a polynucleotide molecule encoding any of the antibodies and/or polypeptides of the aforementioned aspects, as well as other aspects as described herein. In some embodiments, an expression vector comprises the polynucleotide molecule. In other embodiments, a host cell comprises the expression vector. In some embodiments, the host cell comprises the polynucleotide molecule. In some embodiments, the host cell is a hybridoma cell line.

In one aspect, the invention provides a method of inhibiting the growth of a tumor in a subject, comprising administering to the subject a therapeutically effective amount of a BMPR-binding agent. In some embodiments, the tumor is a solid tumor. In some embodiments, the tumor is a colorectal tumor, a breast tumor, a prostate tumor, a pancreatic tumor, a lung tumor, a glioblastoma tumor, a head and neck tumor or a melanoma tumor. In certain embodiments, the tumor comprises cancer stem cells. In certain embodiments, the BMPR-binding agent inhibits growth of the tumor by reducing the number and/or frequency of cancer stem cells in the tumor. In certain embodiments, the BMPR-binding agent is an antibody, such as an antibody that specifically binds at least one BMPR. In some embodiments, the BMPR is a type I receptor or a type II receptor, or a combination thereof. In some embodiments, the subject is a human.

In another aspect, the invention provides a method of reducing the tumorigenicity of a tumor comprising cancer stem cells by reducing the frequency of cancer stem cells in the tumor, wherein the method comprises contacting the tumor with an effective amount of a BMPR-binding agent. In certain embodiments, the agent is an antibody, such as an antibody that specifically binds at least one BMPR. In some embodiments, the BMPR-binding agent modulates the activity of the BMP pathway. In some embodiments, the BMPR-binding agent modulates the activity of a BMPR. In some embodiments, the modulation of BMPR activity stimulates or increases BMP pathway activity. In some embodiments, the modulation of a BMPR activity stimulates or increase BMP pathway signaling.

In another aspect, the invention provides a binding agent (e.g., an antibody) that specifically binds a BMPR and has an effect on cancer stem cells. In some embodiments, the BMP-binding agent reduces the frequency of cancer stem cells in a tumor, reduces the number of cancer stem cells in a tumor, reduces the tumorigenicity of a tumor, and/or reduces the tumorigenicity of a tumor by reducing the number and/or frequency of cancer stem cells in the tumor. In certain embodiments, the antibody specifically binds BMPR1A. In some embodiments, the antibody specifically binds BMPR1B. In some embodiments, the antibody specifically binds BMPR2. In some embodiments, the antibody specifically binds ACVR2A. In some embodiments, the antibody specifically binds ACVR2B.

In certain embodiments of each of the aforementioned aspects, as well as other aspects described elsewhere herein, the tumors which are targeted are breast, colorectal, hepatic, renal, lung, pancreatic, ovarian, prostate, brain, or head and neck tumors.

In another aspect, the invention provides a method of treating cancer in a subject. In some embodiments, the method comprises administering to a subject a BMPR-binding agent. In some embodiments, the method comprises administering to a subject a therapeutically effective amount of any of the antibodies or polypeptides or agents described in the aforementioned aspects, as well as other aspects and embodiments described elsewhere herein. In some embodiments, the cancer to be treated is breast cancer, colorectal cancer, hepatic cancer, kidney cancer, liver cancer, lung cancer, pancreatic cancer, gastrointestinal cancer, melanoma, ovarian cancer, prostate cancer, cervical cancer, bladder cancer, glioblastoma, and head and neck cancer.

In certain embodiments of each of the aforementioned aspects, as well as other aspects described elsewhere herein, the treatment methods further comprise administering at least one additional therapeutic agent appropriate for effecting combination therapy (e.g., a chemotherapeutic agent or other anticancer agent, if cancer is to be treated).

Pharmaceutical compositions comprising both a BMPR-binding agent as described herein and a pharmaceutically acceptable vehicle are further provided, as are cell lines that produce the BMPR-binding agents. Methods of treating cancer and/or inhibiting tumor growth in a subject (e.g., a human) comprising administering to the subject an effective amount of a composition comprising the BMPR-binding agents are also provided.

Where aspects or embodiments of the invention are described in terms of a Markush group or other grouping of alternatives, the present invention encompasses not only the entire group listed as a whole, but also each member of the group individually and all possible subgroups of the main group, and also the main group absent one or more of the group members. The present invention also envisages the explicit exclusion of one or more of any of the group members in the claimed invention.

### BRIEF DESCRIPTIONS OF THE DRAWINGS

FIG. 1. The impact of BMP4 over-expression on the growth of nine primary human tumors in mice. Primary human tumor cells were transduced with a lentiviral vector containing a CMV-BMP4-IRES-GFP expression cassette. GFP-positive cells were sorted and injected subcutaneously in the flank of NOD/SCID mice. Tumor volume was monitored weekly for BMP4-induced inhibition of tumor growth. A vector expressing only GFP was used as a negative control. (A) Responsive breast tumors (B) Responsive colon tumors (C) Non-responsive tumors.

FIG. 2. BMP4 treatment of pre-established colon tumors. NOD/SCID mice bearing ˜150-mm³ pre-established primary human colon tumors were treated with BMP4. 1×10⁹ pfus of AdBMP4 (squares) or Ad-Fc (circles) were administered to the tumor-bearing mice. Tumor growth was monitored for 11 days and mean tumor volumes were plotted as a function of time.

FIG. 3. FACS analysis of BMP4-treated colon tumors. BMP4-treated (FIG. 3B) and control Fc-treated (FIG. 3A) colon tumor cells were analyzed by FACS for ESA, CD44 and CD166 expression. Results are expressed as fluorescence intensities (3A and 3B) or as percentage of positive cells (3C).

FIG. 4. In vivo limiting dilution assay to determine the CSC frequency in BMP4-treated colon tumors. BMP4-treated and control Fc-treated colon tumor cells were tested for tumorigenicity in vivo. Cells were serially diluted to doses of 30, 90 and 270 cells. Individual tumors were measured 56 days post-injection. (A) Tumor volumes of control Fc-treated cells (circles) or BMP4-treated cells (squares) are plotted as a function of injected cell number. (B) Calculated CDC frequencies and error bars are plotted for the control Fc-treated (black) and BMP4-treated (grey) groups.

FIG. 5. BMP4 dose response in OMP-C18 colon xenograft model. 3.5×10⁸ (▪), 1.75×10⁸ (▴), 8.75×10⁷ (▾), 4.38×10⁷ (♦), 2.19×10⁷ (◯) and 1.09×10⁷ (□) pfus of AdBMP4 were injected into NOD/SCID mice on day 29 post-tumor cell injection. 3.5×10⁸ pfus of AdFc () were injected to the control group. 3 tumor measurements were taken after the injection. (A) Tumor averages and corresponding standard errors were plotted for each group as a function of time. (B) The final mouse weights were averaged per group and reported on a bar graph. (C) Percentages of ESA-positive and CD44-positive cells were calculated in 3 different gates, ESA+CD44+, CD44 High and CD44 Low. Treatment group averages and standard errors were plotted in a bar graph. (D) Representative dot plots for a control tumor and a 1.75×10⁸ pfu AdBMP4 tumor are shown side by side. The CD44 high and low cells were gated to highlight the changes within the CD44-positive population.

FIG. 6. Effect of anti-BMPR1A antibody 5M107 on BMP4-induced gene expression. TaqMan qPCR was performed RNA isolated from treated cells. Expression levels are expressed as Log 10 (relative quantity) along the y axis. (A) C2C12 cells were treated with BMP4, BMP4+BMPR1A-Fc and BMP4+BMPR1A-Fc+antibody 5M107. (B) Saos2 cells were treated with control antibody and anti-BMPR1A antibody 5M107. (+) indicates addition of the regent. (−) indicates the absence of the reagent.

FIG. 7. Effect of BMPR1A blockade on colon tumor growth. OMP-C18 tumor-bearing NOD/SCID mice were treated once weekly with the anti-BMPR1A antibody 5M107 or control antibody LZ1. Growth curves were established, and the tumors were analyzed for cell surface marker expression, and CSC frequency. (A) Tumor growth was monitored weekly, and the tumor volume averages and standard errors calculated for anti-BMPR1A antibody 5M107-treated mice (circles) and LZ1-treated mice (squares) were plotted as a function of time. (B) Percentages of ESA-positive and CD44-positive tumor cells were measured by FACS and are shown for control antibody LZ1 (black) and anti-BMPR1A antibody 5M107 (grey). (C) The volumes of each individual LZ1-treated tumor (circles) and anti-BMPR1A antibody 5M107-treated tumors (squares) resulting from the limiting dilution assay were plotted as a function of the number of re-injected cells.

FIG. 8. Effect of BMP activation and BMPR1A blockade on gene expression in colon tumor cells. Total RNA was extracted from whole tumors treated with a BMP4 Adenoviral vector or treated with an anti-BMPR1A antibody (5M107). Gene expression profiles were established for both samples using Affymetrix microarray technology. Two separate gene lists were established that contain the genes regulated 2-fold and more with a p value of at least 0.05 relative to their respective controls. A subset of these genes is shown that demonstrate the opposite impact of BMP4 over-expression and BMPR1A blockade on BMP target genes. Red is up-regulated and green is down-regulated.

FIG. 9. BMPR2 expression levels from primary human colon tumors. Total RNA was extracted from whole tumors. Gene expression profiles were established for each tumor type sample using Affymetrix microarray technology. The data corresponding to 2 different BMPR2 probes were extracted and are shown.

FIG. 10. Report cell lines. Stable mouse C2c12 and human HepG2 cells containing the BRE-Luc reporter were tested for their response to BMP4. Luciferase activity was plotted as a function of BMP4 concentration for 2 C2C12 clones before and after freezing (FIG. 10A) and 1 HepG2 clone before and after freezing (FIG. 10B). The specificity of the reporter system was evaluated in C2C12 clone #56, using increasing amounts of the anti-human BMPR1A antibody 5M107 to a mixture with BMP4 and BMPR1A-Fc decoy receptor.

### DETAILED DESCRIPTION OF THE INVENTION

The present invention provides bone morphogenetic protein receptor (BMPR) binding agents, such as antibodies, and compositions comprising the binding agents. The BMPR-binding agents include agonists of the BMP signaling pathway. The invention also provides methods of using the binding agents to treat cancer. Details of binding agents, compositions, and methods are provided herein.

It has been demonstrated that over-expression of BMP4 inhibits tumor growth in vivo in several xenograft models (Example 1). Treatment with BMP4 systematically delivered by an adenovirus vector inhibited tumor growth in vivo in a colon xenograft model, and a dose response curve demonstrated effective and non-toxic doses (Examples 2 and 5). FACS analysis showed that the percentage of ESA^{high} cells and CD44+CD166+cells was reduced in BMP4-treated tumor cells as compared to control treated tumor cells, suggesting a decrease in the number or frequency of CSC. In addition, limiting dilution analysis of BMP4-treated colon tumor cells demonstrated a 5-fold decrease in CSC frequency as compared to control-treated tumor cells (Examples 3 and 4). Microarray analysis showed that BMP4 over-expression effected gene expression, including several BMP target genes demonstrating that the BMP was activated by BMP4 over-expression (Example 6). Monoclonal antibodies that specifically bind a BMPR have been identified, including anti-BMPR1A antibody 5M107 (Example 7). Antibody 5M107 was shown to inhibit tumor growth in vivo in a xenograft model (Example 9). Microarray analysis demonstrated that antibody 5M107 effected gene expression in treated tumor cells, but in an opposite pattern as compared to cells treated with BMP4 (Example 9).

### I. DEFINITIONS

To facilitate an understanding of the present invention, a number of terms and phrases are defined below.

The terms “bone morphogenetic protein”, “bone morphogenic protein” and “BMP” are used interchangeably.

The terms “agonist” and “agonistic” as used herein refer to or describe a molecule which is capable of, directly or indirectly, substantially inducing, promoting, increasing or enhancing the biological activity of a target and/or a signaling pathway (e.g., the BMP pathway).

The terms “antagonist” and “antagonistic” as used herein refer to any molecule that partially or fully blocks, inhibits, reduces or neutralizes a biological activity of a target and/or signaling pathway (e.g., the BMP pathway). The term “antagonist” is used herein to include any molecule that partially or fully blocks, inhibits, reduces or neutralizes the activity of a protein (e.g., a BMP receptor). Suitable antagonist molecules specifically include antagonist antibodies or antibody fragments.

The terms “modulation” and “modulate” as used herein refer to a change or an alteration in a biological activity. Modulation includes, but is not limited to, stimulating or inhibiting an activity. Modulation may be an increase or a decrease in activity (e.g., protein signaling, pathway signaling), a change in binding characteristics, or any other change in the biological, functional, or immunological properties associated with the activity of a protein, pathway, or other biological point of interest.

The term “antibody” as used herein refers to an immunoglobulin molecule that recognizes and specifically binds a target, such as a protein, polypeptide, peptide, carbohydrate, polynucleotide, lipid, or combinations of the foregoing, through at least one antigen recognition site within the variable region of the immunoglobulin molecule. As used herein, the term encompasses intact polyclonal antibodies, intact monoclonal antibodies, antibody fragments (such as Fab, Fab′, F(ab′)2, and Fv fragments), single chain Fv (scFv) antibodies, multispecific antibodies such as bispecific antibodies generated from at least two intact antibodies, monospecific antibodies, monovalent antibodies, chimeric antibodies, humanized antibodies, human antibodies, fusion proteins comprising an antigen determination portion of an antibody, and any other modified immunoglobulin molecule comprising an antigen recognition site as long as the antibodies exhibit the desired biological activity. An antibody can be any of the five major classes of immunoglobulins: IgA, IgD, IgE, IgG, and IgM, or subclasses (isotypes) thereof (e.g., IgG1, IgG2, IgG3, IgG4, IgA1 and IgA2), based on the identity of their heavy-chain constant domains referred to as alpha delta, epsilon, gamma, and mu, respectively. The different classes of immunoglobulins have different and well-known subunit structures and three-dimensional configurations. Antibodies can be naked or conjugated to other molecules, including but not limited to, toxins and radioisotopes.

The term “antibody fragment” refers to a portion of an intact antibody and refers to the antigenic determining variable regions of an intact antibody. Examples of antibody fragments include, but are not limited to, Fab, Fab′, F(ab′)2, and Fv fragments, linear antibodies, single chain antibodies, and multispecific antibodies formed from antibody fragments.

The term “variable region” of an antibody refers to the variable region of the antibody light chain or the variable region of the antibody heavy chain, either alone or in combination. The variable regions of the heavy and light chain each consist of four framework regions (FR) connected by three complementarity determining regions (CDRs), also known as “hypervariable regions”. The CDRs in each chain are held together in close proximity by the framework regions and, with the CDRs from the other chain, contribute to the formation of the antigen-binding site of antibodies. There are at least two techniques for determining CDRs: (1) an approach based on cross-species sequence variability (i.e., Kabat et al., 1991, Sequences of Proteins of Immunological Interest, 5th ed., National Institutes of Health, Bethesda Md.), and (2) an approach based on crystallographic studies of antigen-antibody complexes (Al-Lazikani et al., 1997, J. Mol. Biol. 273:927-948). In addition, combinations of these two approaches are sometimes used in the art to determine CDRs.

The term “monoclonal antibody” as used herein refers to a homogenous antibody population involved in the highly specific recognition and binding of a single antigenic determinant or epitope. This is in contrast to polyclonal antibodies that typically include a mixture of different antibodies directed against different antigenic determinants. The term “monoclonal antibody” encompasses both intact and full-length monoclonal antibodies as well as antibody fragments (e.g., Fab, Fab′, F(ab′)2, Fv), single chain (scFv) antibodies, fusion proteins comprising an antibody portion, and any other modified immunoglobulin molecule comprising an antigen recognition site. Furthermore, “monoclonal antibody” refers to such antibodies made by any number of techniques, including but not limited to, hybridoma production, phage selection, recombinant expression, and transgenic animals.

The term “humanized antibody” as used herein refers to forms of non-human (e.g., murine) antibodies that are specific immunoglobulin chains, chimeric immunoglobulins, or fragments thereof that contain minimal non-human sequences.

The term “human antibody” as used herein refers to an antibody produced by a human or an antibody having an amino acid sequence corresponding to an antibody produced by a human made using any of the techniques known in the art. This definition of a human antibody specifically excludes a humanized antibody comprising non-human antigen-binding residues.

The term “chimeric antibody” as used herein refers to an antibody wherein the amino acid sequence of the immunoglobulin molecule is derived from two or more species. Typically, the variable region of both light and heavy chains corresponds, to the variable region of antibodies derived from one species of mammals (e.g., mouse, rat, rabbit, etc.) with the desired specificity, affinity, and/or capability, while the constant regions are homologous to the sequences in antibodies derived from another species (usually human) to avoid eliciting an immune response in that species.

The phrase “affinity matured antibody” as used herein refers to an antibody with one or more alterations in one or more CDRs thereof that result in an improvement in the affinity of the antibody for antigen, compared to a parent antibody that does not possess those alterations(s). Preferred affinity matured antibodies will have nanomolar or even picomolar affinities for the target antigen. Affinity matured antibodies are produced by procedures known in the art. For example, Marks et al., Bio/Technology 10:779-783 (1992), describes affinity maturation by VH and VL domain shuffling. Random mutagenesis of CDR and/or framework residues is described by: Barbas et al., 1994, PNAS, 91:3809-3813; Schier et al. 1995, Gene, 169:147-155; Yelton et al., 1995, J. Immunol. 155:1994-2004; Jackson et al., 1995, J. Immunol., 154:3310-9; and Hawkins et al, 1992, J. Mol. Biol., 226:889-896.

The terms “epitope” and “antigenic determinant” are used interchangeably herein and refer to that portion of an antigen capable of being recognized and specifically bound by a particular antibody. When the antigen is a polypeptide, epitopes can be formed both from contiguous amino acids and noncontiguous amino acids juxtaposed by tertiary folding of a protein. Epitopes formed from contiguous amino acids (also referred to as linear epitopes) are typically retained upon protein denaturing, whereas epitopes formed by tertiary folding (also referred to as conformational epitopes) are typically lost upon protein denaturing. An epitope typically includes at least 3, and more usually, at least 5 or 8-10 amino acids in a unique spatial conformation.

The terms “selectively binds” or “specifically binds” mean that a binding agent or an antibody reacts or associates more frequently, more rapidly, with greater duration, with greater affinity, or with some combination of the above to the epitope, protein or target molecule than with alternative substances, including unrelated proteins. In certain embodiments “specifically binds” means, for instance, that an antibody binds a protein with a Kd of about 0.1 mM or less, but more usually less than about 1 μM. In certain embodiments, “specifically binds” means that an antibody binds a target at times with a Kd of at least about 0.1 μM or less and at other times at least about 0.01 μM or less. Because of the sequence identity between homologous proteins in different species, specific binding can include an antibody that recognizes a protein (e.g., BMPR1A) in more than one species. Likewise, because of homology within certain regions of polypeptide sequences of different proteins, specific binding can include an antibody (or other polypeptide or binding agent) that recognizes more than one protein (e.g., human BMPR1A and human BMPR1B). It is understood that, in certain embodiments, an antibody or binding moiety that specifically binds a first target may or may not specifically bind to a second target. As such, “specific binding” does not necessarily require (although it can include) exclusive binding, i.e. binding to a single target. Thus, an antibody may, in certain embodiments, specifically bind to more than one target. In certain embodiments, the multiple targets may be bound by the same antigen-binding site on the antibody. For example, an antibody may, in certain instances, comprise two identical antigen-binding sites, each of which specifically binds the same epitope on two or more proteins (e.g., BMPR1A and BMPR1B). In certain alternative embodiments, an antibody may be bispecific and comprise at least two antigen-binding sites with differing specificities. By way of non-limiting example, a bispecific antibody may comprise one antigen-binding site that recognizes an epitope on one protein (e.g., human BMPR1A) and further comprises a second, different antigen-binding site that recognizes a different epitope on a second protein (e.g., human BMPR2). Generally, but not necessarily, reference to binding means specific binding.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification, such as conjugation with a labeling component. Also included within the definition are, for example, polypeptides containing one or more analogs of an amino acid (including, for example, unnatural amino acids), as well as other modifications known in the art. It is understood that, because the polypeptides of this invention are based upon antibodies, in certain embodiments, the polypeptides can occur as single chains or associated chains.

The terms “polynucleotide” and “nucleic acid,” are used interchangeably herein and refer to polymers of nucleotides of any length, and include DNA and RNA. The nucleotides can be deoxyribonucleotides, ribonucleotides, modified nucleotides or bases, and/or their analogs, or any substrate that can be incorporated into a polymer by DNA or RNA polymerase.

“Conditions of high stringency” may be identified by those that: (1) employ low ionic strength and high temperature for washing, for example 15 mM sodium chloride/1.5 mM sodium citrate/0.1% sodium dodecyl sulfate at 50° C.; (2) employ during hybridization a denaturing agent, such as formamide, for example, 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C.; or (3) employ 50% formamide, 5×SSC (0.75M NaCl, 75 mM sodium citrate), 50 mM sodium phosphate (pH 6.8), 0.1% sodium pyrophosphate, 5×Denhardt's solution, sonicated salmon sperm DNA (50 μg/ml), 0.1% SDS, and 10% dextran sulfate at 42° C., with washes at 42° C. in 0.2×SSC and 50% formamide at 55° C., followed by a high-stringency wash consisting of 0.1×SSC containing EDTA at 55° C.

The terms “identical” or percent “identity” in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity may be measured using sequence comparison software or algorithms or by visual inspection. Various algorithms and software that may be used to obtain alignments of amino acid or nucleotide sequences are well-known in the art. These include but are not limited to, BLAST, ALIGN, Megalign, BestFit, GCG Wisconsin Package, etc. In some embodiments, two nucleic acids or polypeptides of the invention are substantially identical, meaning they have at least 70%, at least 75%, at least 80%, at least 85%, at least 90%, and in some embodiments at least 95%, 96%, 97%, 98%, 99% nucleotide or amino acid residue identity, when compared and aligned for maximum correspondence, as measured using a sequence comparison algorithm or by visual inspection. In some embodiments, identity exists over a region of the sequences that is at least about 10, at least about 20, at least about 40-60 residues in length or any integral value therebetween. In some embodiments, identity exists over a longer region than 60-80 residues, such as at least about 90-100 residues, and in some embodiments the sequences are substantially identical over the full length of the sequences being compared, such as the coding region of a nucleotide sequence.

A “conservative amino acid substitution” is one in which one amino acid residue is replaced with another amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). For example, substitution of a phenylalanine for a tyrosine is a conservative substitution. Preferably, conservative substitutions in the sequences of the polypeptides and antibodies of the invention do not abrogate the binding of the polypeptide or antibody containing the amino acid sequence, to the antigen(s), i.e., the one or more BMPR protein(s) to which the polypeptide or antibody binds. Methods of identifying nucleotide and amino acid conservative substitutions which do not eliminate antigen binding are well-known in the art.

The term “vector” as used herein means a construct, which is capable of delivering, and usually expressing, one or more gene(s) or sequence(s) of interest in a host cell. Examples of vectors include, but are not limited to, viral vectors, naked DNA or RNA expression vectors, plasmid, cosmid, or phage vectors, DNA or RNA expression vectors associated with cationic condensing agents, and DNA or RNA expression vectors encapsulated in liposomes.

A polypeptide, antibody, polynucleotide, vector, cell, or composition which is “isolated” is a polypeptide, antibody, polynucleotide, vector, cell, or composition which is in a form not found in nature. Isolated polypeptides, antibodies, polynucleotides, vectors, cell or compositions include those which have been purified to a degree that they are no longer in a form in which they are found in nature. In some embodiments, an antibody, polynucleotide, vector, cell, or composition which is isolated is substantially pure.

The term “substantially pure” as used herein refers to material which is at least 50% pure (i.e., free from contaminants), at least 90% pure, at least 95% pure, at least 98% pure, or at least 99% pure.

The terms “cancer” and “cancerous” as used herein refer to or describe the physiological condition in mammals in which a population of cells are characterized by unregulated cell growth. Examples of cancer include, but are not limited to, carcinoma, blastoma, sarcoma, and hematologic cancers such as lymphoma and leukemia.

The terms “tumor” and “neoplasm” as used herein refer to any mass of tissue that results from excessive cell growth or proliferation, either benign (noncancerous) or malignant (cancerous) including pre-cancerous lesions.

The terms “proliferative disorder” and “proliferative disease” refer to disorders associated with abnormal cell proliferation such as cancer.

The term “metastasis” as used herein refers to the process by which a cancer spreads or transfers from the site of origin to other regions of the body with the development of a similar cancerous lesion at the new location. A “metastatic” or “metastasizing” cell is one that loses adhesive contacts with neighboring cells and migrates via the bloodstream or lymph from the primary site of disease to invade neighboring body structures.

The terms “cancer stem cell” and “CSC” and “tumor stem cell” are used interchangeably herein and refer to cells from a cancer that: (1) have extensive proliferative capacity; 2) are capable of asymmetric cell division to generate one or more kinds of differentiated progeny with reduced proliferative or developmental potential; and (3) are capable of symmetric cell divisions for self-renewal or self-maintenance. These properties confer on the cancer stem cells the ability to form or establish a tumor or cancer upon serial transplantation into an immunocompromised host (e.g., a mouse) compared to the majority of tumor cells that fail to form tumors. Cancer stem cells undergo self-renewal versus differentiation in a chaotic manner to form tumors with abnormal cell types that can change over time as mutations occur.

The terms “cancer cell” and “tumor cell” refer to the total population of cells derived from a cancer or tumor or pre-cancerous lesion, including both non-tumorigenic cells, which comprise the bulk of the cancer cell population, and tumorigenic stem cells (cancer stem cells). As used herein, the terms “cancer cell” or “tumor cell” will be modified by the term “non-tumorigenic” when referring solely to those cells lacking the capacity to renew and differentiate to distinguish those tumor cells from cancer stem cells.

The term “tumorigenic” as used herein refers to the functional features of a cancer stem cell including the properties of self-renewal (giving rise to additional tumorigenic cancer stem cells) and proliferation to generate all other tumor cells (giving rise to differentiated and thus non-tumorigenic tumor cells).

The term “tumorigenicity” as used herein of a tumor refers to the ability of a random sample of cells from the tumor to form palpable tumors upon serial transplantation into immunocompromised hosts (e.g., mice).

The term “subject” refers to any animal (e.g., a mammal), including, but not limited to, humans, non-human primates, canines, felines, rodents, and the like, which is to be the recipient of a particular treatment. Typically, the terms “subject” and “patient” are used interchangeably herein in reference to a human subject.

The term “pharmaceutically acceptable” refers to approved or approvable by a regulatory agency of the Federal or a state government or listed in the U.S. Pharmacopeia or other generally recognized pharmacopeia for use in animals, including humans.

The terms “pharmaceutically acceptable excipient, carrier or adjuvant” or “acceptable pharmaceutical carrier” refer to an excipient, carrier or adjuvant that can be administered to a subject, together with at least one binding agent (e.g., an antibody) of the present disclosure, and which does not destroy the pharmacological activity thereof and is nontoxic when administered in doses sufficient to deliver a therapeutic effect.

The terms “effective amount” or “therapeutically effective amount” or “therapeutic effect” refer to an amount of a binding agent, an antibody, polypeptide, polynucleotide, small organic molecule, or other drug effective to “treat” a disease or disorder in a subject or mammal. In the case of cancer, the therapeutically effective amount of a drug (e.g., an antibody) has a therapeutic effect and as such can reduce the number of cancer cells; decrease tumorigenicity, tumorigenic frequency or tumorigenic capacity; reduce the number or frequency of cancer stem cells; reduce the tumor size; reduce the cancer cell population; inhibit or stop cancer cell infiltration into peripheral organs including, for example, the spread of cancer into soft tissue and bone; inhibit and stop tumor or cancer cell metastasis; inhibit and stop tumor or cancer cell growth; relieve to some extent one or more of the symptoms associated with the cancer; reduce morbidity and mortality; improve quality of life; or a combination of such effects. To the extent the agent, for example an antibody, prevents growth and/or kills existing cancer cells, it can be referred to as cytostatic and/or cytotoxic.

The terms “treating” or “treatment” or “to treat” or “alleviating” or “to alleviate” refer to both 1) therapeutic measures that cure, slow down, lessen symptoms of, and/or halt progression of a diagnosed pathologic condition or disorder and 2) prophylactic or preventative measures that prevent or slow the development of a targeted pathologic condition or disorder. Thus those in need of treatment include those already with the disorder; those prone to have the disorder; and those in whom the disorder is to be prevented. In some embodiments, a subject is successfully “treated” according to the methods of the present invention if the patient shows one or more of the following: a reduction in the number of or complete absence of cancer cells; a reduction in the tumor size; inhibition of or an absence of cancer cell infiltration into peripheral organs including the spread of cancer cells into soft tissue and bone; inhibition of or an absence of tumor or cancer cell metastasis; inhibition or an absence of cancer growth; relief of one or more symptoms associated with the specific cancer; reduced morbidity and mortality; improvement in quality of life; reduction in tumorigenicity; reduction in the number or frequency of cancer stem cells; or some combination of effects.

As used in the present disclosure and claims, the singular forms “a”, “an”, and “the” include plural forms unless the context clearly dictates otherwise.

It is understood that wherever embodiments are described herein with the language “comprising” otherwise analogous embodiments described in terms of “consisting of” and/or “consisting essentially of” are also provided.

The term “and/or” as used in a phrase such as “A and/or B” herein is intended to include both “A and B,” “A or B,” “A” and “B.” Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### II. BONE MORPHOGENETIC PROTEIN RECEPTOR (BMPR)-BINDING AGENTS

The present invention provides agents that specifically bind at least one BMPR (e.g., BMPR1A, BMPR1B, BMPR2, ACVR2A and/or ACVR2B). These agents are referred to herein as “BMPR-binding agents”. In certain embodiments, the agents bind BMPR1A. In certain embodiments, the agents bind BMPR1B. In certain embodiments, the agents bind BMPR2. In certain embodiments, the agents bind ACVR2A. In certain embodiments, the agents bind ACVR2B. In certain embodiments, the agents bind more than one BMPR. In certain embodiments, the BMPR is a human BMPR (hBMPR). The full-length amino acid (aa) sequences for human BMPR1A, BMPR1B, BMPR2, ACVR2A and ACVR2B are known in the art and are provided herein as SEQ ID NO:1 (BMPR1A aa), SEQ ID NO:2 (BMPR2 aa), SEQ ID NO:3 (BMPR1B aa), SEQ ID NO:4 (ACVR2A aa) and SEQ ID NO:5 (ACVR2B aa). In certain embodiments, the BMPR is a mouse BMPR (mBMPR). The full-length amino acid (aa) sequences for mouse BMPR1A, BMPR1B, BMPR2, ACVR2A and ACVR2B are known in the art and are provided herein as SEQ ID NO:6 (mBMPR1A aa), SEQ ID NO:7 (mBMPR1B aa), SEQ ID NO:8 (mACVR2A aa), SEQ ID NO:9 (mACVR2B aa), and SEQ ID NO:10 (mBMPR2).

In some embodiments, the BMPR-binding agent is an antibody. In some embodiments, the BMPR-binding agent is an antibody that specifically binds at least one BMPR. In some embodiments, the BMPR is a human BMPR. In certain embodiments, the BMPR-binding agent is an antibody that specifically binds BMPR1A. In certain embodiments, the binding agent is an antibody that specifically binds BMPR1B. In some embodiments, the binding agent is an antibody that specifically binds BMPR2. In some embodiments, the binding agent is an antibody that specifically binds ACVR2A. In some embodiments, the binding agent is an antibody that specifically binds ACVR2B. In some embodiments, the binding agent is a bispecific antibody that specifically binds two BMPRs. In some embodiments, the bispecific antibody binds a type I BMPR and a type II BMPR. In certain embodiments, the bispecific antibody binds BMPR1A and a type II BMPR. In certain embodiments, the bispecific antibody binds BMPR and a type II BMPR. In certain embodiments, the bispecific antibody binds BMPR1A and BMPR2. In certain embodiments, the bispecific antibody binds BMPR1A and ACVR2A. In certain embodiments, the bispecific antibody binds BMPR1A and ACVR2B. In certain embodiments, the bispecific antibody binds BMPR1B and BMPR2. In certain embodiments, the bispecific antibody binds BMPR1B and ACVR2A. In certain embodiments, the bispecific antibody binds BMPR1B and ACVR2B.

In certain embodiments, the BMPR-binding agent (e.g., an antibody) specifically binds the extracellular domain (ECD) of a BMPR. In some embodiments, the BMPR-binding agent binds a specific region within the extracellular domain. In certain embodiments, the BMPR-binding agent is an bispecific antibody that binds the extracellular domains of two BMPRs.

In certain embodiments, the BMPR-binding agent (e.g., an antibody) binds an extracellular domain of a BMPR with a dissociation constant (K_{D}) of about 1 μM or less, about 100 nM or less, about 40 nM or less, about 20 nM or less, about 10 nM or less, or about 1 nM or less. In certain embodiments, the BMPR-binding agent or antibody binds a human BMPR with a K_{D} of about 40 nM or less, about 20 nM or less, about 10 nM or less, or about 1 nM or less. In some embodiments, the dissociation constant of the binding agent or antibody to a particular BMPR is the dissociation constant determined using a BMPR fusion protein comprising a BMPR extracellular domain (e.g., a BMPR1A ECD-Fc fusion protein) immobilized on a Biacore chip.

In certain embodiments, the BMPR-binding agent (e.g., an antibody) binds a BMPR with a half maximal effective concentration (EC₅₀) of about 1 μM or less, about 100 nM or less, about 40 nM or less, about 20 nM or less, about 10 nM or less, or about 1 nM or less.

In certain embodiments, the BMPR-binding agent is a polypeptide. In certain embodiments, the BMPR-binding agent or polypeptide is an antibody. In certain embodiments, the antibody is an IgG antibody. In some embodiments, the antibody is an IgG1 antibody, in some embodiments, the antibody is an IgG2 antibody. In certain embodiments, the antibody is a monoclonal antibody. In certain embodiments, the antibody is a humanized antibody. In certain embodiments, the antibody is a human antibody. In certain embodiments, the antibody is an antibody fragment. In certain embodiments, the antibody is a bispecific antibody.

The BMPR-binding agents (e.g., antibodies) of the present invention can be assayed for specific binding by any method known in the art. The immunoassays which can be used include, but are not limited to, competitive and non-competitive assay systems using techniques such as Biacore analysis, FACS analysis, immunofluorescence, immunocytochemistry, Western blot analysis, radioimmunoassay, ELISA, “sandwich” immunoassay, immunoprecipitation assay, precipitation reaction, gel diffusion precipitin reaction, immunodiffusion assay, agglutination assay, complement-fixation assay, immunoradiometric assay, fluorescent immunoassay, and protein A immunoassay. Such assays are routine and well known in the art (see, e.g., Ausubel et al., eds, 1994, Current Protocols in Molecular Biology, Vol. 1, John Wiley & Sons, Inc., New York).

In some embodiments, the specific binding of a BMPR-binding agent (e.g., an antibody) to a human BMPR may be determined using ELISA. An ELISA assay comprises preparing a BMPR antigen, coating wells of a 96 well microtiter plate with antigen, adding to the wells the BMPR-binding agent or antibody conjugated to a detectable compound such as an enzymatic substrate (e.g., horseradish peroxidase or alkaline phosphatase), incubating for a period of time and detecting the presence of the binding agent or antibody. In some embodiments, the BMPR-binding agent or antibody is not conjugated to a detectable compound, but instead a second conjugated antibody that recognizes the BMPR-binding agent or antibody is added to the well. In some embodiments, instead of coating the well with a BMPR antigen, the BMPR-binding agent or antibody can be coated to the well, antigen is added to the coated well and then a second antibody conjugated to a detectable compound is added. One of skill in the art would be knowledgeable as to the parameters that can be modified and/or optimized to increase the signal detected, as well as other variations of ELISAs that can be used (see e.g., Ausubel et al., 1994, Current Protocols in Molecular Biology, Vol. 1, John Wiley & Sons, Inc., New York at 11.2.1).

The binding affinity of an antibody or other binding agent to a BMPR and the on-off rate of an antibody-antigen interaction can be determined by competitive binding assays. In some embodiments, a competitive binding assay is a radioimmunoassay comprising the incubation of labeled antigen (e.g., ³H- or ¹²⁵I-labeled antigen), or fragment or variant thereof, with the antibody of interest in the presence of increasing amounts of unlabeled antigen followed by the detection of the antibody bound to the labeled antigen. The affinity of the antibody for the antigen and the on-off rates can be determined from the data by Scatchard plot analysis. In some embodiments, Biacore kinetic analysis is used to determine the binding affinities and on-off rates of antibodies or agents that bind a BMPR (e.g., BMPR1A, BMPR1B, BMPR2, ACVR2A, ACVR2B). Biacore kinetic analysis comprises analyzing the binding and dissociation of antibodies from antigens (e.g., BMPR proteins) that have been immobilized on the surface of a Biacore chip. In some embodiments, Biacore kinetic analyses can be used to study binding of different antibodies in qualitative epitope competitive binding assays.

In certain embodiments, the invention provides an antibody that specifically binds an extracellular domain of a human BMPR, wherein the antibody comprises one, two, three, four, five and/or six of the CDRs of antibody 5M107. In some embodiments, the antibody comprises one or more of the CDRs of 5M107, two or more of the CDRs of 5M107, three or more of the CDRs of 5M107, four or more of the CDRs of 5M107, five or more of the CDRs of 5M107, or all six of the CDRs of 5M107. In certain embodiments, the polypeptide comprises one, two or three of the CDRs from the heavy chain variable region of 5M107. In some embodiments, the polypeptide comprises one, two or three of the CDRs from the heavy chain variable region of 5M107 and CDRs from the light chain variable region of an antibody different than 5M107. In some embodiments, the antibody comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In certain embodiments, the invention provides an antibody that specifically binds an extracellular domain of human BMPR1A, wherein the antibody comprises: a heavy chain CDR1 comprising TGYYMK (SEQ ID NO:14), a heavy chain CDR2 comprising RINPDNGGRTYNQIFKDK (SEQ ID NO:15), and a heavy chain CDR3 comprising RERGQYGNYGGFSD (SEQ ID NO:16).

In certain embodiments, the invention provides an antibody that specifically binds an extracellular domain of human BMPR1A, wherein the antibody comprises (a) a heavy chain CDR1 comprising TGYYMK (SEQ ID NO:14), or a variant thereof comprising 1, 2, 3, or 4 amino acid substitutions; (b) a heavy chain CDR2 comprising RINPDNGGRTYNQIFKDK (SEQ ID NO:15), or a variant thereof comprising 1, 2, 3, or 4 amino acid substitutions; and (c) a heavy chain CDR3 comprising RERGQYGNYGGFSD (SEQ ID NO:16), or a variant thereof comprising 1, 2, 3, or 4 amino acid substitutions. In some embodiments, the amino acid substitutions are conservative substitutions.

In certain embodiments, the invention provides an antibody that specifically binds an extracellular domain of human BMPR1A, wherein the antibody comprises a heavy chain variable region having at least about 80% sequence identity to SEQ ID NO:12 or SEQ ID NO:13. In certain embodiments, the antibody comprises a heavy chain variable region having at least about 85%, at least about 90%, at least about 95%, at least about 97%, or at least about 99% sequence identity to SEQ ID NO:12 or SEQ ID NO:13. In some embodiments, the antibody is a monoclonal antibody or antibody fragment. In some embodiments, the antibody is a bispecific antibody, wherein the antibody comprises one heavy chain variable region having at least about 80% sequence identity to SEQ ID NO:12 or SEQ ID NO:13.

In some embodiments, the BMPR-binding agent is an antibody, 5M107, produced by the hybridoma cell line (5M107.1) deposited with the ATCC under the conditions of the Budapest Treaty on Mar. 17, 2010 and assigned number PTA-10720. In some embodiments, the antibody is a humanized version of 5M107. In one embodiment, the BMPR-binding agent comprises, consists essentially of, or consists of, an anti-BMPR1A antibody which is a 5M107 IgG antibody.

In other embodiments, the invention provides an antibody that competes with any of the antibodies as described in the aforementioned embodiments and/or aspects, as well as other aspects/embodiments described elsewhere herein, for specific binding to the extracellular domain of a human BMPR.

The invention provides a variety of polypeptides, including but not limited to, antibodies and fragments of antibodies. In certain embodiments, the polypeptide is isolated. In certain alternative embodiments, the polypeptide is substantially pure.

In certain embodiments, the BMPR-binding agent (e.g., an antibody) binds the same epitope that an antibody comprising the heavy chain variable region comprising SEQ ID NO:13 binds. In some embodiments, the BMPR-binding agent or antibody binds the same epitope as the 5M107 antibody. In some embodiments, the BMPR-binding agent (e.g., antibody) binds an overlapping epitope.

In certain embodiments, the BMPR-binding agent competes for specific binding to an extracellular domain of a human BMPR with an antibody, wherein the antibody comprises a heavy chain variable region comprising SEQ ID NO:13. In some embodiments, the BMPR-binding agent or antibody competes for specific binding to an extracellular domain of a human BMPR in a competitive binding assay.

In certain embodiments, the BMPR-binding agent competes with antibody 5M107 for specific binding to human BMPR1A. In some embodiments, the BMPR-binding agent or antibody competes for specific binding to an extracellular domain of human BMPR1A in a competitive binding assay.

The invention provides polypeptides, including, but not limited to, antibodies that specifically bind to a human BMPR. In certain embodiments, the polypeptide comprises one, two, three, four, five and/or six of the CDRs of antibody 5M107. In some embodiments, the polypeptide comprises one or more of the CDRs of 5M107, two or more of the CDRs of 5M107, three or more of the CDRs of 5M107, four or more of the CDRs of 5M107, five or more of the CDRs of 5M107, or all six of the CDRs or 5M107. In certain embodiments, the polypeptide comprises one, two or three of the CDRs from the heavy chain variable region of 5M107. In some embodiments, the polypeptide comprises CDRs with up to four (i.e., 0, 1, 2, 3, or 4) amino acid substitutions per CDR. In certain embodiments, the heavy chain CDR(s) are contained within a heavy chain variable region. In certain embodiments, the light chain CDR(s) are contained within a light chain variable region.

In some embodiments, the invention provides a polypeptide that specifically binds a human BMPR, wherein the polypeptide comprises: an amino acid sequence having at least about 80% sequence identity to SEQ ID NO:12 or SEQ ID NO:13. In certain embodiments, the polypeptide comprises an amino acid sequence having at least about 85%, at least about 90%, at least about 95%, at least about 97%, or at least about 99% sequence identity to SEQ ID NO:12 or SEQ ID NO:13. In certain embodiments, the polypeptide comprises an amino acid sequence having at least about 95% sequence identity to SEQ ID NO:12 or SEQ ID NO:13. In certain embodiments, the polypeptide comprises an amino acid sequence comprising SEQ ID NO:12 or SEQ ID NO:13. In certain embodiments, the polypeptide specifically binds BMPR1A.

Polypeptides comprising one of the individual light chains or heavy chains described herein, as well as polypeptides (e.g., antibodies) comprising both a light chain and a heavy chain described herein are also provided.

In certain embodiments, the BMPR-binding agent comprises the heavy chain and light chain of the 5M107 IgG2 antibody (with or without the leader sequence). In certain embodiments, the BMPR-binding agent comprises the heavy chain of the 5M107 IgG2 antibody (with or without the leader sequence). In certain embodiments, the BMPR-binding agent is the 5M107 IgG2 antibody. The hybridoma cell line (5M107.1) producing the 5M107 IgG2 antibody was deposited with the American Type Culture Collection (ATCC), 10801 University Boulevard, Manassas, Va., USA, under the conditions of the Budapest Treaty on Mar. 17, 2010 and assigned ATCC deposit designation number PTA-10720.

In certain embodiments, the BMPR-binding agent is an agent that competes for specific binding to BMPR1A with an antibody produced by the cell line deposited with ATCC as 5M107.1 (e.g., in a competitive binding assay).

In certain embodiments, the BMPR-binding agent (e.g., an antibody) binds a BMPR and modulates BMP pathway activity. In some embodiments, the BMPR-binding agent is an agonist and modulates BMP pathway activity. In some embodiments, the BMPR-binding agent is an antagonist and modulates BMP pathway activity.

In certain embodiments, the BMPR-binding agent (e.g., an antibody) binds a BMPR and modulates BMPR activity. In some embodiments, the BMPR-binding agent is an agonist and modulates BMPR activity. In some embodiments, the BMPR-binding agent is an antagonist and modulates BMPR activity.

In certain embodiments, the BMPR-binding agent (e.g., an antibody) is an agonist of a human BMPR (e.g., BMPR1A, BMPR1B, BMPR2, ACVR2A, and/or ACVR2B). In certain embodiments, the BMPR-binding agent is an agonist and stimulates BMPR activity. In some embodiments, the BMPR-binding agent is an agonist and increases BMPR activity. In some embodiments, the BMPR-binding agent is an agonist and stimulates BMP pathway activity. In some embodiments, the BMPR-binding agent stimulates human BMPR1A activity. In some embodiments, the BMPR-binding agent stimulates human BMPR1B activity. In some embodiments, the BMPR-binding agent stimulates human BMPR2 activity. In some embodiments, the BMPR-binding agent stimulates human ACVR2A activity. In some embodiments, the BMPR-binding agent stimulates human ACVR2B activity. In certain embodiments, the BMPR-binding agent stimulates and/or increases at least about 10%, at least about 20%, at least about 30%, at least about 50%, at least about 75%, at least about 90%, or about 100% of the activity of the bound human BMPR.

In certain embodiments, the BMPR-binding agent (e.g., an antibody) is an antagonist of a human BMFR (e.g., BMPR1A, BMPR1B, BMPR2, ACVR2A, and/or ACVR2B). In some embodiments, the BMPR-binding agent is an antagonist of a BMPR and inhibits BMPR activity. In some embodiments, the BMPR-binding agent is an antagonist of a BMPR and inhibits BMP pathway activity. In some embodiments, the BMPR-binding agent inhibits human BMPR1A activity. In some embodiments, the BMPR-binding agent inhibits human BMPR1B activity. In some embodiments, the BMPR-binding agent inhibits human ACVR1B activity. In some embodiments, the BMPR-binding agent inhibits human ACVR1C activity. In some embodiments, the BMPR-binding agent inhibits human BMPR2 activity. In some embodiments, the BMPR-binding agent inhibits human ACVR2A activity. In some embodiments, the BMPR-binding agent inhibits human ACVR2B activity. In certain embodiments, the BMPR-binding agent inhibits at least about 10%, at least about 20%, at least about 30%, at least about 50%, at least about 75%, at least about 90%, or about 100% of the activity of the bound human BMPR.

In vivo and in vitro assays for determining whether a BMPR-binding agent (or candidate BMPR-binding agent) inhibits or stimulates the BMP pathway are known in the art. For example in some embodiments, a cell-based, luciferase reporter assay utilizing a BRE-Luc (BMP Responsive Element-Luciferase) reporter vector containing the BMP responsive elements of the mouse Id1 gene upstream of a firefly luciferase reporter gene may be used to measure BMP signaling levels in vitro. The BRE-Luc construct comprises two copies of the Id1-(−1105/−1080) fragment fused to two copies of the Id1-(−1052/−1032) fragment cloned upstream of a minimal promoter. Cells are transfected with the BRE-Luc reporter vector, cells are plated in 96-well plates and incubated overnight. Cells are washed and incubated in media containing BMPR-binding agents or positive/negative controls. After 8-24 hours the cells are lysed, mixed with Luciferase Assay Reagent (Promega) and luminescence is measured using a luminometer. The level of BMPR activation and/or BMP pathway activation induced by a BMPR-binding agent is compared to the level of BMPR activation in the absence of a BMPR-binding agent. In some embodiments, the cells are transiently transfected. In some embodiments, the cells are stably transfected. In some embodiments, the BRE-Luc reporter vector is transfected into mouse C2C12 or human HepG2 cells. In certain embodiments, cells transfected with BRE-Luc reporter vector are used to screen for BMPR agonist antibodies.

Mouse C2C12 and human HepG2 cell lines are known to be responsive to BMPs. Expression levels of each receptor (e.g., BMPR1A, BMPR1B, BMPR2, ACVR2A and ACVR2B) can be evaluated in these cell lines by quantitative PCR. shRNAs can be used to knock-down expression of each BMPR and evaluate the loss of expression on BMP pathway function. In some embodiments, mouse C2C12 and/or human HepG2 cell lines will be used to identify BMPR-binding agents.

In certain embodiments, the BMPR-binding agents (e.g., antibodies) have one or more of the following effects: inhibit proliferation of tumor cells, inhibit tumor cell growth, prevent or reduce metastasis of tumor cells, reduce the frequency of cancer stem cells in a tumor, trigger cell death of tumor cells (e.g., by apoptosis), reduce the tumorigenicity of tumor cells by reducing the frequency of cancer stem cells in the tumor cell population, differentiate tumorigenic cells to a non-tumorigenic state, or increase survival of a patient.

In certain embodiments, the BMPR-binding agents (e.g., antibodies) have one or more of the following effects: inhibit proliferation of cancer cells, inhibit cancer cell growth, prevent or reduce metastasis of cancer cells, reduce the frequency of cancer stem cells in a cancer, trigger cell death of cancer cells (e.g., by apoptosis), reduce the tumorigenicity of cancer cells by reducing the frequency of cancer stem cells in the cancer cell population, differentiate tumorigenic cells to a non-tumorigenic state, or increase survival of a patient.

In certain embodiments, the BMPR-binding agents (e.g., antibodies) are capable of inhibiting tumor cell growth. In certain embodiments, the BMPR-binding agents are capable of inhibiting growth of tumor cells in vitro (e.g., contacting tumor cells with an antibody in vitro). In certain embodiments, the BMPR-binding agents are capable of inhibiting tumor growth in vivo (e.g., in a xenograft mouse model and/or in a human having a tumor).

In certain embodiments, the BMPR-binding agents (e.g., antibodies) are capable of inhibiting cancer cell growth. In certain embodiments, the BMPR-binding agents are capable of inhibiting growth of cancer cells in vitro (e.g., contacting cancer cells with an antibody in vitro). In certain embodiments, the BMPR-binding agents are capable of inhibiting cancer growth in vivo (e.g., in a xenograft mouse model and/or in a human having cancer).

The invention further provides methods of inhibiting the growth of a tumor by administering the BMPR-binding agents to a subject with a tumor. The invention further provides methods of treating cancer by administering the BMPR-binding agents to a subject in need thereof. In some embodiments, the methods of treating cancer or inhibiting tumor growth comprise targeting cancer stem cells with the BMPR-binding agents. In certain embodiments, the methods comprise reducing the frequency of cancer stern cells in a tumor, reducing the number of cancer stem cells in a tumor, reducing the tumorigenicity of a tumor, and/or reducing the tumorigenicity of a tumor by reducing the number or frequency of cancer stem cells in the tumor. The invention also provides methods of using the BMPR-binding agents in the treatment of cancer and/or in the inhibition of the growth of tumors comprising cancer stem cells.

In certain embodiments, the BMPR-binding agents (e.g., antibodies) are capable of reducing the tumorigenicity of a solid tumor. In certain embodiments, the BMPR-binding agent or antibody is capable of reducing the tumorigenicity of a solid tumor comprising cancer stem cells in an animal model, such as a mouse xenograft model. In some embodiments, the BMPR-binding agent is capable of reducing the tumorigenicity of a solid tumor by reducing the frequency of cancer stem cells in the tumor. In certain embodiments, the number or frequency of cancer stem cells in a tumor is reduced by at least about two-fold, about three-fold, about five-fold, about ten-fold, about 50-fold, about 100-fold, or about 1000-fold. In certain embodiments, the reduction in the frequency of cancer stem cells is determined by a limiting dilution assay (LDA) using an animal model. Examples and guidance regarding the use of limiting dilution assays to determine a reduction in the number or frequency of cancer stem cells in a tumor can be found, e.g., in International Pub. No. WO 2008/042236 and U.S. Patent Application Pub. Nos. 2008/0064049 and 2008/0178305.

In certain embodiments, BMPR-binding agents or antibodies mediate cell death of a cell expressing a BMPR via antibody-dependent cellular cytotoxicity (ADCC). ADCC involves cell lysis by effector cells that recognize the Fc portion of an antibody. Many lymphocytes, monocytes, tissue macrophages, granulocytes and eosinophils, for example, have Fc receptors and can mediate cytolysis (Dillman, 1994, J. Clin. Oncol. 12:1497).

In some embodiments, BMPR-binding agents or antibodies trigger cell death of a cell expressing a BMPR by activating complement-dependent cytotoxicity (CDC). CDC involves binding of serum complement to the Fc portion of an antibody and subsequent activation of the complement protein cascade, resulting in cell membrane damage and eventual cell death. Biological activity of antibodies is known to be determined, to a large extent, by the constant domains or Fc region of the antibody molecule (Uananue and Benacerraf, 1984, Textbook of Immunology, 2nd Edition, Williams & Wilkins, p. 218). Antibodies of different classes and subclasses differ in this respect, as do antibodies of the same subclass but from different species. Of human antibodies, IgM is the most efficient class of antibodies to bind complement, followed by IgG1, IgG3, and IgG2 whereas IgG4 appears quite deficient in activating the complement cascade (Dillman, 1994, J. Clin. Oncol. 12:1497; Jefferis et al., 1998, Immunol. Rev. 163:59-76). According to the present invention, antibodies of those classes having the desired biological activity can be prepared.

The ability of any particular BMPR-binding agent or antibody to mediate lysis of the target cell by CDC and/or ADCC can be assayed. In some embodiments, the cells of interest are grown and labeled in vitro (target cells) and the antibody is added to the cell culture in combination with either serum complement or immune cells which can be activated by the antigen antibody complexes. Cytolysis of the target cells is detected, for example, by the release of label from the lysed cells. In some embodiments, antibodies can be screened using a patient's own serum as a source of complement and/or immune cells. The antibody that is capable of activating complement or mediating ADCC in the in vitro test can then be used therapeutically in that particular patient.

In certain embodiments, the BMPR-binding agent (e.g., an antibody) has a circulating half-life in a subject or mammal (e.g., mice, rats, cynomolgus monkeys, or humans) of at least about 5 hours, at least about 10 hours, at least about 24 hours, at least about 3 days, at least about 1 week, or at least about 2 weeks. In certain embodiments, the BMPR-binding agent is an IgG (e.g., IgG1 or IgG2) antibody that has a circulating half-life in a subject or mammal (e.g., mice, rats, cynomolgus monkeys, or humans) of at least about 5 hours, at least about 10 hours, at least about 24 hours, at least about 3 days, at least about 1 week, or at least about 2 weeks. Methods of increasing the half-life of agents such as polypeptides and antibodies are known in the art. In some embodiments, known methods of increasing the circulating half-life of IgG antibodies include the introduction of mutations in the Fc region which increase the pH-dependent binding of the antibody to the neonatal Fc receptor (FcRn) at pH 6.0 (see e.g., U.S. Patent Pub. Nos. 2005/0276799; 2007/0148164; and 2007/0122403). Known methods of increasing the circulating half-life of antibody fragments lacking the Fc region include, but are not limited to, techniques such as PEGylation.

In some embodiments, the BMPR-binding agents are polyclonal antibodies. Polyclonal antibodies can be prepared by any known method. In some embodiments, polyclonal antibodies are raised by immunizing an animal (e.g. a rabbit, rat, mouse, goat, donkey) by multiple subcutaneous or intraperitoneal injections of the relevant antigen (e.g., a purified peptide fragment, full-length recombinant protein, or fusion protein). The antigen can be optionally conjugated to a carrier such as keyhole limpet hemocyanin (KLH) or serum albumin. The antigen (with or without a carrier protein) is diluted in sterile saline and usually combined with an adjuvant (e.g., Complete or Incomplete Freund's Adjuvant) to form a stable emulsion. After a sufficient period of time, polyclonal antibodies are recovered from blood, ascites and the like, of the immunized animal. The polyclonal antibodies can be purified from serum or ascites according to standard methods in the art including, but not limited to, affinity chromatography, ion-exchange chromatography, gel electrophoresis, and dialysis.

In some embodiments, the BMPR-binding agents are monoclonal antibodies. Monoclonal antibodies can be prepared using hybridoma methods known to one of skill in the art (see e.g., Kohler and Milstein, 1975, Nature 256:495-497). In some embodiments, using the hybridoma method, a mouse, hamster, or other appropriate host animal, is immunized as described above to elicit from lymphocytes the production of antibodies that will specifically bind the immunizing antigen. In some embodiments, lymphocytes can be immunized in vitro. In some embodiments, the immunizing antigen can be a human protein or a portion thereof. In some embodiments, the immunizing antigen can be a mouse protein or a portion thereof. In some embodiments, the immunizing agent is the ECD, or a portion thereof, of a human BMPR. In some embodiments, the immunizing agent is the ECD, or a portion thereof, of a mouse BMPR.

Following immunization, lymphocytes are isolated and fused with a suitable myeloma cell line using, for example, polyethylene glycol, to form hybridoma cells that can then be selected away from unfused lymphocytes and myeloma cells. Hybridomas that produce monoclonal antibodies directed specifically against a chosen antigen may be identified by a variety of methods including, but not limited to, immunoprecipitation, immunoblotting, and in vitro binding assay (e.g., flow cytometry, enzyme-linked immunosorbent assay (ELISA), and radioimmunoassay (RIA)). The hybridomas can be propagated either in in vitro culture using, standard methods (Goding, Monoclonal Antibodies: Principles and Practice, Academic Press, 1986) or in in vivo as ascites tumors in an animal. The monoclonal antibodies can be purified from the culture medium or ascites fluid according to standard methods in the art including, but not limited to, affinity chromatography, ion-exchange chromatography, gel electrophoresis, and dialysis.

In certain embodiments, monoclonal antibodies can be made using recombinant DNA techniques as known to one skilled in the art (see e.g., U.S. Pat. No. 4,816,567). The polynucleotides encoding a monoclonal antibody are isolated from mature B-cells or hybridoma cells, such as by RT-PCR using oligonucleotide primers that specifically amplify the genes encoding the heavy and light chains of the antibody, and their sequence is determined using conventional techniques. The isolated polynucleotides encoding the heavy and light chains are then cloned into suitable expression vectors which produce the monoclonal antibodies when transfected into host cells such as E. coli, simian COS cells, Chinese hamster ovary (CHO) cells, or myeloma cells that do not otherwise produce immunoglobulin protein. In other embodiments, recombinant monoclonal antibodies, or fragments thereof, can be isolated from phage display libraries expressing CDRs of the desired species (see e.g., McCafferty et al., 1990, Nature, 348:552-554; Clackson et al., 1991, Nature, 352:624-628; and Marks et al., 1991, J. Mol. Biol., 222:581-597).

The polynucleotide(s) encoding a monoclonal antibody can further be modified using recombinant DNA technology to generate alternative antibodies. In some embodiments, the constant domains of the light and heavy chains of, for example, a mouse monoclonal antibody can be substituted 1) for those regions of, for example, a human antibody to generate a chimeric antibody or 2) for a non-immunoglobulin polypeptide to generate a fusion antibody. In other embodiments, the constant regions are truncated or removed to generate the desired antibody fragment of a monoclonal antibody. Site-directed or high-density mutagenesis of the variable region can be used to optimize specificity, affinity, and/or other biological characteristics of a monoclonal antibody. In some embodiments, site-directed mutagenesis of the CDRs can be used to optimize specificity, affinity, and/or other biological characteristics of a monoclonal antibody.

In some embodiments, the BMPR-binding agent is a humanized antibody. Typically, humanized antibodies are human immunoglobulins in which residues from the CDRs are replaced by residues from a CDR of a non-human species (e.g., mouse, rat, rabbit, hamster, etc.) that have the desired specificity, affinity, and/or capability using methods known to one skilled in the art. In some embodiments, the Fv framework region residues of a human immunoglobulin are replaced with the corresponding residues in an antibody from a non-human species that has the desired specificity, affinity, and/or capability. In some embodiments, the humanized antibody can be further modified by the substitution of additional residues either in the Fv framework region and/or within the replaced non-human residues to refine and optimize antibody specificity, affinity, and/or capability. In general, the humanized antibody will comprise substantially all of at least one, and typically two or three, variable domains containing all, or substantially all, of the CDR regions that correspond to the non-human immunoglobulin whereas all, or substantially all, of the framework regions are those of a human immunoglobulin consensus sequence. In some embodiments, the humanized antibody can also comprise at least a portion of an immunoglobulin constant region or domain (Fc), typically that of a human immunoglobulin. In certain embodiments, such humanized antibodies are used therapeutically because they may reduce antigenicity and HAMA (human anti-mouse antibody) responses when administered to a human subject. One skilled in the art would be able to obtain a functional humanized antibody with reduced immunogenicity following known techniques (see e.g., U.S. Pat. Nos. 5,225,539; 5,585,089; 5,693,761; and 5,093,762).

In certain embodiments, the BMPR-binding agent is a human antibody. Human antibodies can be directly prepared using various techniques known in the art. In some embodiments, immortalized human lymphocytes immunized in vitro or isolated from an immunized individual that produces an antibody directed against a target antigen can be generated (see, e.g., Cole et al., Monoclonal Antibodies and Cancer Therapy, Alan R. Liss, p. 77 (1985); Boerner et al., 1991, J. Immunol., 147 (1):86-95; and U.S. Pat. Nos. 5,750,373; 5,567,610 and 5,229,275). In some embodiments, the human antibody can be selected from a phage library, where that phage library expresses human antibodies (Vaughan et al., 1996, Nature Biotechnology, 14:309-314; Sheets et al., 1998, PNAS, 95:6157-6162; Hoogenboom and Winter, 1991, J. Biol., 227:381; Marks et al., 1991, J. Mol. Biol., 222:581). Alternatively, phage display technology can be used to produce human antibodies and antibody fragments in vitro, from immunoglobulin variable (V) domain gene repertoires from unimmunized donors. Phage display technology can be used to produce human antibodies and antibody fragments in vitro from immunoglobulin variable (V) domain gene repertoires from synthetic libraries. Techniques for the generation and use of antibody phage libraries are also described in U.S. Pat. Nos. 5,969,108; 6,172,197; 5,885,793; 6,521,404; 6,544,731; 6,555,313; 6,582,915; 6,593,081; 6,300,064; 6,653,068; 6,706,484; and 7,264,963; and Rothe et al., 2008, J. Mol. Bio., 376:1182-1200. Affinity maturation strategies, such as chain shuffling (Marks et al., 1992, Bio/Technology, 10:779-783), are known in the art and may be employed to generate high affinity human antibodies.

In some embodiments, human antibodies can be made in transgenic mice containing human immunoglobulin loci that are capable, upon immunization, of producing the full repertoire of human antibodies in the absence of endogenous immunoglobulin production. This approach is described in U.S. Pat. Nos. 5,545,807; 5,545,806; 5,569,825; 5,625,126; 5,633,425; and 5,661,016.

In some embodiments, the BMPR-binding agent is a bispecific antibody. Bispecific antibodies are antibodies that are capable of specifically recognizing and binding at least two different epitopes, or have “dual specificity” (see, e.g., Wu et al., 2007, Nature Biotech., 25:1290-97). The different epitopes can either be within the same molecule (e.g., a BMPR1A) or on different molecules (e.g., BMPR1A and BMPR2) such that both, for example, can be specifically recognized and bound by the antibody. Bispecific antibodies can be intact antibodies or antibody fragments. In some embodiments, the bispecific antibodies are monoclonal human or humanized antibodies. In some embodiments, the antibodies can specifically recognize and bind a first antigen target, (e.g., a type I BMPR) as well as a second antigen target (e.g., a type II BMPR). In certain embodiments, a bispecific antibody specifically binds BMPR1A, as well as at least one additional BMPR selected from the group consisting of BMPR2, ACVR2A and ACVR2B. In certain embodiments, a bispecific antibody specifically binds BMPR1B, as well as at least one additional BMPR selected from the group consisting of BMPR2, ACVR2A and ACVR2B. In some embodiments, a bispecific antibody binds BMPR1A and BMPR2. In some embodiments, a bispecific antibody binds BMPR1B and BMPR2.

Antibodies with a dual specificity in their binding arms usually do not occur in nature and, therefore, have been developed through recombinant DNA or cell-fusion technology. Some bispecific antibodies were designed to recruit cytotoxic effector cells of the immune system effectively against pathogenic target cells. These antibodies possess an antigen-binding arm and an arm which binds a cytotoxic agent or a radionuclide chelator, such as EOTUBE, DPTA, DOTA, or TETA. Other bispecific antibodies were designed to redirect T cells against cancer target cells. Target cells are killed when cytotoxic T lymphocytes (CTLs) are tethered to tumor cells and simultaneously triggered by one arm of the bispecific antibody that interact with the T-cell receptor (TCR)-CD3 complex. CTLs, which are considered to be the most potent killer cells of the immune system, cannot be engaged by monoclonal antibodies because they lack Fey-receptors.

Techniques for making bispecific antibodies are known to those skilled in the art, see for example, Millstein et al., 1983, Nature, 305:537-539; Brennan et al., 1985, Science, 229:81; Suresh et al., 1986, Methods in Enzymol., 121:120; Traunecker et al., 1991, EMBO J., 10:3655-3659; Shalaby et al., 1992, J. Exp. Med., 175:217-225; Kostelny et al., 1992, J. Immunol., 148:1547-1553; Gruber et al., 1994, J. Immunol., 152:5368; and U.S. Pat. No. 5,731,168). Bispecific antibodies can be intact antibodies or antibody fragments. Antibodies with more than two valencies are also contemplated. For example, trispecific antibodies can be prepared (Tat et al., 1991, J. Immunol., 147:60). Thus, in certain embodiments the antibodies to BMPR are multispecific.

One method for generating bispecific antibodies has been termed the “knobs-into-holes” strategy (see, e.g., WO 2006/028936). The mispairing of Ig heavy chains is reduced in this technology by mutating selected amino acids forming the interface of the CH3 domains in human IgG. At positions within the CH3 domain at which the two heavy chains interact directly, an amino acid with a small side chain (hole) is introduced into the sequence of one heavy chain and an amino acid with a large side chain (knob) into that of the other one. As a result, the protein interaction between knobs and holes has been described as leading to the formation of up to 90% of the correct bispecific human IgG by transfected mammalian host cells.

Another method for generating bispecific antibodies comprises mutating selected amino acids that interact at the interface between two immunoglobulin CH3 domain-containing polypeptides by replacing an amino acid residue involved in hydrophilic interactions with a more hydrophobic amino acid residue and/or replacing an amino acid involved in a charge interaction with another amino acid. This technique utilizes the novel amino acid substitution within the interface region of the CH3 domain of the antibody constant domain to create a pair of variant antibody heavy chains that favor heterodimerization. In some embodiments, by using an invariant light chain and two heavy chain variable regions targeting distinct targets, it is possible to generate unique bispecific antibodies. (See, e.g. U.S. Ser. No. 12/768,650, hereby incorporated by reference in its entirety).

In certain embodiments, the BMPR-binding agent or antibody described herein may be monospecific. For example, in certain embodiments, each of the one or more antigen-binding sites that an antibody contains is capable of binding (or binds) a homologous epitope on BMPR. In certain embodiments, an antigen-binding site of a monospecific antibody described herein is capable of binding (or binds) BMPR1A and a second BMPR such as BMPR1B (i.e., the same epitope is found on BMPR1A and, for example, on BMPR1B).

In certain embodiments, the BMPR-binding agent is an antibody fragment. Antibody fragments may have different functions or capabilities than intact antibodies; for example, antibody fragments can have increased tumor penetration. Various techniques are known for the production of antibody fragments including, but not limited to, proteolytic digestion of intact antibodies. In some embodiments, antibody fragments include a F(ab′)2 fragment produced by pepsin digestion of an antibody molecule. In some embodiments, antibody fragments include a Fab fragment generated by reducing the disulfide bridges of an F(ab′)2 fragment. In other embodiments, antibody fragments include a Fab fragment generated by the treatment of the antibody molecule with papain and a reducing agent. In certain embodiments, antibody fragments are produced recombinantly. In some embodiments, antibody fragments include Fv or single chain Fv (scFv) fragments. Fab, Fv, and scFv antibody fragments can be expressed in and secreted from E. coli or other host cells, allowing for the production of large amounts of these fragments. In some embodiments, antibody fragments are isolated from antibody phage libraries as discussed herein. For example, methods can be used for the construction of Fab expression libraries (Huse et al., 1989, Science, 246:1275-1281) to allow rapid and effective identification of monoclonal Fab fragments with the desired specificity for a BMPR protein or derivatives, fragments, analogs or homologs thereof. In some embodiments, antibody fragments are linear antibody fragments as described in U.S. Pat. No. 5,641,870. In certain embodiments, antibody fragments are monospecific or bispecific. In certain embodiments, the BMPR-binding agent is a scFv. Various techniques can be used for the production of single-chain antibodies specific to a BMPR (see, e.g., U.S. Pat. No. 4,946,778).

It can further be desirable, especially in the case of antibody fragments, to modify an antibody in order to increase its serum half-life. This can be achieved, for example, by incorporation of a salvage receptor binding epitope into the antibody fragment by mutation of the appropriate region in the antibody fragment or by incorporating the epitope into a peptide tag that is then fused to the antibody fragment at either end or in the middle (e.g., by DNA or peptide synthesis).

For the purposes of the present invention, it should be appreciated that modified antibodies, or fragments thereof, can comprise any type of variable region that provides for the association of the antibody with the extracellular domain of a BMPR. In this regard, the variable region may be derived from any type of mammal that can be induced to mount a humoral response and generate immunoglobulins against a desired antigen (e.g., a BMPR). As such, the variable region of the modified antibodies can be, for example, of human, murine, non-human primate (e.g., cynomolgus monkeys, macaques, etc.) or rabbit origin. In some embodiments, both the variable and constant regions of the modified immunoglobulins are human. In other embodiments, the variable regions of compatible antibodies (usually derived from a non-human source) can be engineered or specifically tailored to improve the binding properties or reduce the immunogenicity of the molecule. In this respect, variable regions useful in the present invention can be humanized or otherwise altered through the inclusion of imported amino acid sequences.

In some embodiments, the variable domains in both the heavy and light chains are altered by at least partial replacement of one or more CDRs and, if necessary, by partial framework region replacement and sequence modification. Although the CDRs may be derived from an antibody of the same class or even subclass as the antibody from which the framework regions are derived, it is envisaged that the CDRs will be derived from an antibody of different class and preferably from an antibody from a different species. It may not be necessary to replace all of the CDRs with all of the CDRs from the donor variable region to transfer the antigen binding capacity of one variable domain to another. Rather, it may only be necessary to transfer those residues that are necessary to maintain the activity of the antigen binding site.

Alterations to the variable region notwithstanding, those skilled in the art will appreciate that the modified antibodies of this invention will comprise antibodies (e.g., full-length antibodies or antigen-binding fragments thereof) in which at least a fraction of one or more of the constant region domains has been deleted or otherwise altered so as to provide desired biochemical characteristics, such as increased cancer cell localization, increased tumor penetration, reduced serum half-life or increased serum half-life, when compared with an antibody of approximately the same immunogenicity comprising a native or unaltered constant region. In some embodiments, the constant region of the modified antibodies comprises a human constant region. Modifications to the constant region include additions, deletions or substitutions of one or more amino acids in one or more domains. The modified antibodies disclosed herein may comprise alterations or modifications to one or more of the three heavy chain constant domains (CH1, CH2 or CH3) and/or to the light chain constant domain (CL). In some embodiments, one or more domains are partially or entirely deleted from the constant regions of the modified antibodies. In other embodiments, the entire CH2 domain is removed (ΔCH2 constructs). In some embodiments, the omitted constant region domain is replaced by a short amino acid spacer (e.g., 10 aa residues) that provides some of the molecular flexibility typically imparted by the absent constant region.

In some embodiments, the modified antibodies are engineered to fuse the CH3 domain directly to the hinge region of the antibody. In other embodiments, a peptide spacer is inserted between the hinge region and the modified CH2 and/or CH3 domains. For example, constructs may be expressed wherein the CH2 domain has been deleted and the remaining CH3 domain (modified or unmodified) is joined to the hinge region with a 5-20 amino acid spacer. Such a spacer may be added to ensure that the regulatory elements of the constant domain remain free and accessible or that the hinge region remains flexible. However, it should be noted that amino acid spacers may, in some cases, prove to be immunogenic and elicit an unwanted immune response against the construct. Accordingly, in certain embodiments, any spacer added to the construct will be relatively non-immunogenic so as to maintain the desired biological qualities of the modified antibodies.

In some embodiments, the modified antibodies may have only a partial deletion of a constant domain or substitution of a few or even a single amino acid. For example, the mutation of a single amino acid in selected areas of the CH2 domain may be enough to substantially reduce Fc binding and thereby increase cancer cell localization and/or tumor penetration. Similarly, it may be desirable to simply delete that part of one or more constant region domains that control a specific effector function (e.g. complement C1q binding) to be modulated. Such partial deletions of the constant regions may improve selected characteristics of the antibody (serum half-life) while leaving other desirable functions associated with the subject constant region domain intact. Moreover, as alluded to above, the constant regions of the disclosed antibodies may be modified through the mutation or substitution of one or more amino acids that enhances the profile of the resulting construct. In this respect it may be possible to disrupt the activity provided by a conserved binding site (e.g., Fc binding) while substantially maintaining the configuration and immunogenic profile of the modified antibody. In certain embodiments, the modified antibodies comprise the addition of one or more amino acids to the constant region to enhance desirable characteristics such as decreasing or increasing effector function or provide for more cytotoxin or carbohydrate attachment.

It is known in the art that the constant region mediates several effector functions. For example, binding of the C1 component of complement to the Fc region of IgG or IgM antibodies (bound to antigen) activates the complement system. Activation of complement is important in the opsonization and lysis of cell pathogens. The activation of complement also stimulates the inflammatory response and can also be involved in autoimmune hypersensitivity. In addition, the Fc region of an antibody can bind to a cell expressing a Fc receptor (FcR). There are a number of Fc receptors which are specific for different classes of antibody, including IgG (gamma receptors), IgE (epsilon receptors), IgA (alpha receptors) and IgM (mu receptors). Binding of antibody to Fc receptors on cell surfaces triggers a number of important and diverse biological responses including engulfment and destruction of antibody-coated particles, clearance of immune complexes, lysis of antibody-coated target cells by killer cells (ADCC), release of inflammatory mediators, placental transfer and control of immunoglobulin production.

In some embodiments, the BMPR-binding agents or antibodies provide for altered effector functions that, in turn, affect the biological profile of the administered antibody. For example, in some embodiments, the deletion or inactivation (through point mutations or other means) of a constant region domain may reduce Fc receptor binding of the circulating modified antibody (e.g., BMPR-antibody) thereby increasing cancer cell localization and/or tumor penetration. In other embodiments, the constant region modifications increase or reduce the serum half-life of the antibody. In some embodiments, the constant region is modified to eliminate disulfide linkages or oligosaccharide moieties allowing for enhanced cancer cell localization.

In certain embodiments, a BMPR-binding agent or antibody does not have one or more effector functions. In some embodiments, the antibody has no ADCC activity, and/or no CDC activity. In certain embodiments, the antibody does not bind to the Fc receptor and/or complement factors. In certain embodiments, the antibody has no effector function.

The present invention further embraces variants and equivalents which are substantially homologous to the chimeric, humanized and/or human antibodies, or antibody fragments thereof, set forth herein. These can contain, for example, conservative substitution mutations, i.e. the substitution of one or more amino acids by similar amino acids.

Thus, the present invention provides methods for generating an antibody that binds a BMPR. In some embodiments, the BMPR that is used to generate an antibody is selected from the group consisting of BMPR1A, BMPR1B, BMPR2, ACVR2A and ACVR2B. In some embodiments, the method for generating an antibody that binds a BMPR comprises using hybridoma techniques. In some embodiments, the method comprises using an extracellular domain of a human or mouse BMPR as an immunizing antigen. In some embodiments, the method of generating an antibody that binds a BMPR comprises screening a human phage library. The present invention further provides methods of identifying an antibody that binds a BMPR. In some embodiments, the antibody is identified by screening for binding to the BMPR with flow cytometry (FACS). In some embodiments, the antibody is identified by screening for stimulation or an increase in BMP pathway activation or signaling. In some embodiments, the antibody is identified by screening for stimulation or an increase of BMPR signaling. In some embodiments, the antibody is identified by screening for inhibition or blocking of BMP pathway activation or signaling. In some embodiments, the antibody is identified by screening for inhibition or blocking of BMPR signaling.

In certain embodiments, the antibodies as described herein are isolated. In certain embodiments, the antibodies as described herein are substantially pure.

In some embodiments of the present invention, the BMPR-binding agents are polypeptides. The polypeptides can be recombinant polypeptides, natural polypeptides, or synthetic polypeptides that bind the extracellular domain of a human BMPR. In some embodiments, the polypeptides comprise an antibody or fragment thereof that binds the extracellular domain of a human BMPR. It will be recognized by those of skill in the art that some amino acid sequences of a polypeptide can be varied without significant effect of the structure or function of the protein. Thus, the BMPR-binding polypeptides further include variations of the polypeptides which show substantial binding activity to the extracellular domain of a human BMPR. In some embodiments, amino acid sequence variations of BMPR-binding polypeptides include deletions, insertions, inversions, repeats, and/or type substitutions.

The polypeptides and variants thereof, can be further modified to contain additional chemical moieties not normally part of the polypeptide. The derivatized moieties can improve the solubility, the biological half life and/or absorption of the polypeptide. The moieties can also reduce or eliminate any undesirable side effects of the polypeptides and variants. An overview for chemical moieties can be found in Remington: The Science and Practice of Pharmacy, 21^{st} Edition, University of the Sciences Philadelphia 2005.

The polypeptides described herein can be produced by any suitable method known in the art. Such methods range from direct protein synthesis methods to constructing a DNA sequence encoding polypeptide sequences and expressing those sequences in a suitable host. In some embodiments, a DNA sequence is constructed using recombinant technology by isolating or synthesizing a DNA sequence encoding a wild-type protein of interest. Optionally, the sequence can be mutagenized by site-specific mutagenesis to provide functional analogs thereof. (See, e.g., Zoeller et al., 1984, PNAS, 81:5662-5066 and U.S. Pat. No. 4,588,585.)

In some embodiments, a DNA sequence encoding a polypeptide of interest may be constructed by chemical synthesis using an oligonucleotide synthesizer. Oligonucleotides can be designed based on the amino acid sequence of the desired polypeptide and by selecting those codons that are favored in the host cell in which the recombinant polypeptide of interest will be produced. Standard methods can be applied to synthesize a polynucleotide sequence encoding a polypeptide of interest. For example, a complete amino acid sequence can be used to construct a back-translated gene. Further, a DNA oligomer containing a nucleotide sequence coding for the particular isolated polypeptide can be synthesized. For example, several small oligonucleotides coding for portions of the desired polypeptide can be synthesized and then ligated. The individual oligonucleotides typically contain 5′ or 3′ overhangs for complementary assembly.

Once assembled (by synthesis, site-directed mutagenesis or another method), the polynucleotide sequences encoding a particular polypeptide of interest can be inserted into an expression vector and operatively linked to an expression control sequence appropriate for expression of the polypeptide in a desired host. Proper assembly can be confirmed by nucleotide sequencing, restriction mapping, and/or expression of a biologically active polypeptide in a suitable host. As is well-known in the art, in order to obtain high expression levels of a transfected gene in a host, the gene must be operatively linked to transcriptional and translational expression control sequences that are functional in the chosen expression host.

In certain embodiments, recombinant expression vectors are used to amplify and express DNA encoding BMPR-binding agents such as polypeptides or antibodies or fragments thereof. For example, recombinant expression vectors can be replicable DNA constructs which have synthetic or cDNA-derived DNA fragments encoding a polypeptide chain of a BMPR-binding agent or antibody or fragment thereof, operatively linked to suitable transcriptional or translational regulatory elements derived from mammalian, microbial, viral or insect genes. A transcriptional unit generally comprises an assembly of (1) a genetic element or elements having a regulatory role in gene expression, for example, transcriptional promoters or enhancers, (2) a structural or coding sequence which is transcribed into mRNA and translated into protein, and (3) appropriate transcription and translation initiation and termination sequences. Regulatory elements can include an operator sequence to control transcription. The ability to replicate in a host, usually conferred by an origin of replication, and a selection gene to facilitate recognition of transformants can additionally be incorporated. DNA regions are “operatively linked” when they are functionally related to each other. For example, DNA for a signal peptide (secretory leader) is operatively linked to DNA for a polypeptide if it is expressed as a precursor which participates in the secretion of the polypeptide; a promoter is operatively linked to a coding sequence if it controls the transcription of the sequence; or a ribosome binding site is operatively linked to a coding sequence if it is positioned so as to permit translation. In some embodiments, structural elements intended for use in yeast expression systems include a leader sequence enabling extracellular secretion of translated protein by a host cell. In other embodiments, where recombinant protein is expressed without a leader or transport sequence, it can include an N-terminal methionine residue. This residue can optionally be subsequently cleaved from the expressed recombinant protein to provide a final product.

The choice of expression control sequence and expression vector depends upon the choice of host. A wide variety of expression host/vector combinations can be employed. Useful expression vectors for eukaryotic hosts include, for example, vectors comprising expression control sequences from SV40, bovine papilloma virus, adenovirus and cytomegalovirus. Useful expression vectors for bacterial hosts include known bacterial plasmids, such as plasmids from E. coli, including pCR1, pBR322, pMB9 and their derivatives, and wider host range plasmids, such as M13 and other filamentous single-stranded DNA phages.

Suitable host cells for expression of a BMPR-binding agent or antibody (or a BMPR polypeptide to use as an antigen) include prokaryotes, yeast, insect or higher eukaryotic cells under the control of appropriate promoters. Prokaryotes include gram-negative or gram-positive organisms, for example, E. coli or Bacillus. Higher eukaryotic cells include established cell lines of mammalian origin as described below. Cell-free translation systems could also be employed.

Various mammalian or insect cell culture systems are used to express recombinant polypeptides. Expression of recombinant proteins in mammalian cells can be performed because such proteins are generally correctly folded, appropriately modified and completely functional. Examples of suitable mammalian host cell lines include COS-7 (monkey kidney-derived), L-929 (murine fibroblast-derived), C127 (murine mammary tumor-derived), 3T3 (murine fibroblast-derived), CHO (Chinese hamster ovary-derived), HeLa (human cervical cancer-derived) and BHK (hamster kidney fibroblast-derived) cell lines. Mammalian expression vectors can comprise non-transcribed elements such as an origin of replication, a suitable promoter and enhancer linked to the gene to be expressed, and other 5′ or 3′ flanking non-transcribed sequences, and 5′ or 3′ non-translated sequences, such as necessary ribosome binding sites, a polyadenylation site, splice donor and acceptor sites, and transcriptional termination sequences. Baculovirus systems for production of heterologous proteins in insect cells are well-known to those of skill in the art (see, e.g. Luckow and Summers, 1988, Bio/Technology, 6:47).

The proteins produced by a transformed host can be purified according to any suitable method. Such standard methods include chromatography (e.g., ion exchange, affinity and sizing column chromatography), centrifugation, differential solubility, or by any other standard technique for protein purification. Affinity tags such as hexahistidine, maltose binding domain, influenza coat sequence and glutathione-S-transferase can be attached to the protein to allow easy purification by passage over an appropriate affinity column. Isolated proteins can also be physically characterized using such techniques as proteolysis, high performance liquid chromatography (HPLC), nuclear magnetic resonance (NMR), and x-ray crystallography.

In some embodiments, supernatants from expression systems which secrete recombinant protein into culture media can be first concentrated using a commercially available protein concentration filter, for example, an Amicon or Millipore Pellicon ultrafiltration unit. Following the concentration step, the concentrate can be applied to a suitable purification matrix. In other embodiments, an anion exchange resin can be employed, for example, a matrix or substrate having pendant diethylaminoethyl (DEAE) groups. The matrices can be acrylamide, agarose, dextran, cellulose or other types commonly employed in protein purification. In some embodiments, a cation exchange step can be employed. Suitable cation exchangers include various insoluble matrices comprising sulfopropyl or carboxymethyl groups. In some embodiments, a hydroxyapatite (CHT) media can be employed, including but not limited to, ceramic hydroxyapatite. In some embodiments, one or more reversed-phase HPLC steps employing hydrophobic RP-HPLC media, e.g., silica gel having pendant methyl or other aliphatic groups, can be employed to further purify a recombinant protein. Some or all of the foregoing purification steps, in various combinations, can also be employed to provide a homogeneous recombinant protein.

In some embodiments, recombinant protein produced in bacterial culture can be isolated, for example, by initial extraction from cell pellets, followed by one or more concentration, salting-out, aqueous ion exchange or size exclusion chromatography steps. HPLC can be employed for final purification steps. Microbial cells employed in expression of a recombinant protein can be disrupted by any convenient method, including freeze-thaw cycling, sonication, mechanical disruption, or use of cell lysing agents.

Methods known in the art for purifying antibodies and other proteins also include, for example, those described in U.S. Patent Appl. Nos. 2008/0312425; 2008/0177048; and 2009/0187005.

In certain embodiments, the BMPR-binding agent is a polypeptide that is not an antibody. A variety of methods for identifying and producing non-antibody polypeptides that bind with high affinity to a protein target are known in the art. See, e.g., Skerra, 2007, Curr. Opin. Biotechnol., 18:295-304; Hosse et al., 2006, Protein Science, 15:14-27; Gill et al., 2006, Curr. Opin. Biotechnol., 17:653-658; Nygren, 2008, FEBS J., 275:2668-76; and Skerra, 2008, FEBS J., 275:2677-83. In certain embodiments, phage display technology may be used to produce and/or identify a BMPR-binding polypeptide. In certain embodiments, the BMPR-binding polypeptide comprises a protein scaffold of a type selected from the group consisting of protein A, protein G, a lipocalin, a fibronectin domain, an ankyrin consensus repeat domain, and thioredoxin.

In certain embodiments, the BMPR-binding agents or antibodies can be used in any one of a number of conjugated (i.e. an immunoconjugate or radioconjugate) or non-conjugated forms. In certain embodiments, the antibodies can be used in a non-conjugated form to harness the subject's natural defense mechanisms including complement-dependent cytotoxicity and antibody dependent cellular toxicity to eliminate the malignant or cancer cells.

In some embodiments, the BMPR-binding agent (e.g., an antibody or polypeptide) is conjugated to a cytotoxic agent. In some embodiments, the cytotoxic agent is a chemotherapeutic agent including, but not limited to, methotrexate, adriamicin, doxorubicin, melphalan, mitomycin C, chlorambucil, daunorubicin or other intercalating agents. In some embodiments, the cytotoxic agent is an enzymatically active toxin of bacterial, fungal, plant or animal origin, or fragments thereof, including, but not limited to, diphtheria A chain, nonbinding active fragments of diphtheria toxin, exotoxin A chain, ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes. In some embodiments, the cytotoxic agent is a radioisotope to produce a radioconjugate or a radioconjugated antibody. A variety of radionuclides are available for the production of radioconjugated antibodies including, but not limited to, ⁹⁰Y, ¹²⁵I, ¹³¹I, ¹²³I, ¹¹¹In, ¹³¹In, ¹⁰⁵Rh, ¹⁵³Sm, ⁶⁷Cu, ⁶⁷Ga, ¹⁶⁶Ho, ¹⁷⁷Lu, ¹⁸⁶Re, ¹⁸⁸Re and ²¹²Bi. Conjugates of an antibody and one or more small molecule toxins, such as a calicheamicin, maytansinoids, a trichothene, and CC1065, and the derivatives of these toxins that have toxin activity, can also be used. Conjugates of an antibody and cytotoxic agent are made using a variety of bifunctional protein-coupling agents such as N-succinimidyl-3-(2-pyridyldithiol) propionate (SPDP), iminothiolane (IT), bifunctional derivatives of imidoesters (such as dimethyl adipimidate HCL), active esters (such as disuccinimidyl suberate), aldehydes (such as glutareldehyde), bis-azido compounds (such as bis(p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene).

Heteroconjugate antibodies are also within the scope of the present invention. Heteroconjugate antibodies are composed of two covalently joined antibodies. Such antibodies have, for example, been proposed to target immune cells to unwanted cells (U.S. Pat. No. 4,676,980). It is contemplated that the antibodies can be prepared in vitro using known methods in synthetic protein chemistry, including those involving crosslinking agents.

### III. POLYNUCLEOTIDES

In certain embodiments, the invention encompasses polynucleotides comprising polynucleotides that encode a polypeptide that specifically binds a BMPR or a fragment of such a polypeptide. The term “polynucleotides that encode a polypeptide” encompasses a polynucleotide which includes only coding sequences for the polypeptide as well as a polynucleotide which includes additional coding and/or non-coding sequences. For example, the invention provides a polynucleotide comprising a nucleic acid sequence that encodes an antibody to a human BMPR or encodes a fragment of such an antibody. The polynucleotides of the invention can be in the form of RNA or in the form of DNA. DNA includes cDNA, genomic DNA, and synthetic DNA; and can be double-stranded or single-stranded, and if single stranded can be the coding strand or non-coding (anti-sense) strand.

In certain embodiments, a polynucleotide comprising a polynucleotide encoding a polypeptide comprising a sequence of SEQ ID NO:12 or SEQ ID NO:13 is provided. In some embodiments, a polynucleotide sequence encoding a polypeptide (with or without the signal sequence) comprising a sequence of SEQ ID NO:12 or SEQ ID NO:13 is provided.

In some embodiments, a polynucleotide comprising the nucleotide sequence (with or without the signal sequence) of SEQ ID NO:11 is provided.

In certain embodiments, a polynucleotide comprising a polynucleotide (with or without the signal sequence) having a nucleotide sequence at least 80% identical, at least 85% identical, at least 90% identical, at least 95% identical, and in some embodiments, at least 96%, 97%, 98% or 99% identical to a polynucleotide comprising the sequence of SEQ ID NO:11 is provided. In some embodiments, the polynucleotides have a nucleotide sequence at least 90% identical to SEQ ID NO:11.

Also provided is a polynucleotide that comprises a polynucleotide that hybridizes to SEQ ID NO:11, and/or to a polynucleotide encoding a polypeptide having the sequence of SEQ ID NO:12 or SEQ ID NO:13. In certain embodiments, the hybridization is under conditions of high stringency.

In certain embodiments, the polynucleotides comprise the coding sequence for the mature polypeptide fused in the same reading frame to a polynucleotide which aids, for example, in expression and secretion of a polypeptide from a host cell (e.g., a leader sequence which functions as a secretory sequence for controlling transport of a polypeptide from the cell). The polypeptide having a leader sequence is a preprotein and can have the leader sequence cleaved by the host cell to produce the mature form of the polypeptide. The polynucleotides can also encode for a proprotein which is the mature protein plus additional 5′ amino acid residues. A mature protein having a prosequence is a proprotein and is an inactive form of the protein. Once the prosequence is cleaved an active mature protein remains.

In certain embodiments, the polynucleotides comprise the coding sequence for the mature polypeptide fused in the same reading frame to a marker sequence that allows, for example, for purification and/or identification of the encoded polypeptide. For example, the marker sequence can be a hexa-histidine tag supplied by a pQE-9 vector to provide for purification of the mature polypeptide fused to the marker in the case of a bacterial host, or the marker sequence can be a hemagglutinin (HA) tag derived from the influenza hemagglutinin protein when a mammalian host (e.g., COS-7 cells) is used. In some embodiments, the marker sequence is a FLAG-tag, a peptide of sequence DYKDDDK (SEQ ID NO:17) which can be used in conjunction with other affinity tags.

The present invention further relates to variants of the hereinabove described polynucleotides encoding, for example, fragments, analogs, and/or derivatives.

In certain embodiments, the present invention provides polynucleotides comprising polynucleotides having a nucleotide sequence at least 80% identical, at least 85% identical, at least 90% identical, at least 95% identical, and in some embodiments, at least 96%, 97%, 98% or 99% identical to a polynucleotide encoding a polypeptide comprising an antibody, or fragment thereof, to a human BMPR described herein.

As used herein, the phrase a polynucleotide having a nucleotide sequence at least, for example, 95% “identical” to a reference nucleotide sequence is intended to mean that the nucleotide sequence of the polynucleotide is identical to the reference sequence except that the polynucleotide sequence can include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence can be deleted or substituted with another nucleotide, or a number of nucleotides up to 5% of the total nucleotides in the reference sequence can be inserted into the reference sequence. These mutations of the reference sequence can occur at the 5′ or 3′ terminal positions of the reference nucleotide sequence or anywhere between those terminal positions, interspersed either individually among nucleotides in the reference sequence or in one or more contiguous groups within the reference sequence.

The polynucleotide variants can contain alterations in the coding regions, non-coding regions, or both. In some embodiments, the polynucleotide variants contain alterations which produce silent substitutions, additions, or deletions, but do not alter the properties or activities of the encoded polypeptide. In some embodiments, polynucleotide variants contain “silent” substitutions due to the degeneracy of the genetic code. Polynucleotide variants can be produced for a variety of reasons, for example, to optimize codon expression for a particular host (e.g., change codons in the human mRNA to those preferred by a bacterial host such as E. coli).

In some embodiments, polynucleotides may comprise modified nucleotides, such as methylated nucleotides and their analogs. If present, modification to the nucleotide structure may be imparted before or after assembly of the polymer. The sequence of nucleotides may be interrupted by non-nucleotide components. A polynucleotide may be further modified after polymerization, such as by conjugation with a labeling component. Other types of modifications include, for example, “caps”; substitution of one or more of the naturally occurring nucleotides with an analog; internucleotide modifications such as uncharged linkages (e.g., methyl phosphonates, phosphotriesters, phosphoamidates, and cabamates) and charged linkages (e.g., phosphorothioates and phosphorodithioates); pendant moieties, such as proteins (e.g., nucleases, toxins, antibodies, signal peptides, and poly-L-lysine); intercalators (e.g., acridine and psoralen); chelators (e.g., metals, radioactive metals, boron, and oxidative metals); alkylators; modified linkages (e.g., alpha anomeric nucleic acids); as well as unmodified forms of the polynucleotide(s). Further, any of the hydroxyl groups ordinarily present in the sugars may be replaced, for example, by phosphonate groups, phosphate groups, protected by standard protecting groups, or activated to prepare additional linkages to additional nucleotides, or may be conjugated to solid supports. The 5′ and 3′ terminal OH can be phosphorylated or substituted with amines or organic capping group moieties of from 1 to 20 carbon atoms. Other hydroxyls may also be derivatized to standard protecting groups. Polynucleotides can also contain analogous forms of ribose or deoxyribose sugars that are generally known in the art, including, for example, 2′-O-methyl-, 2′-O-allyl, 2′-fluoro- or 2′-azido-ribose, carbocyclic sugar analogs, alpha-anomeric sugars, epimeric sugars such as arabinose, xyloses or lyxoses, pyranose sugars, furanose sugars, heptuloses, acyclic analogs and abasic nucleoside analogs such as methyl riboside. One or more phosphodiester linkages may be replaced by alternative linking groups. These alternative linking groups include, but are not limited to, embodiments wherein phosphate is replaced by P(O)S (“thioate”), P(S)S (“dithioate”), (O)NR₂ (“amidate”), P(O)R, P(O)OR′, CO or CH₂ (“formacetal”), in which each R or R′ is independently H or a substituted or unsubstituted alkyl (1-20 C) optionally containing an ether (—O—) linkage, aryl, alkenyl, cycloalkyl, cycloalkenyl or araldyl. Not all linkages in a polynucleotide need be identical.

In certain embodiments, the polynucleotides as described herein are isolated. In certain embodiments, the polynucleotides as described herein are substantially pure.

Vectors and cells comprising the polynucleotides described herein are also provided. In some embodiments, an expression vector comprises a polynucleotide molecule. In some embodiments, a host cell comprises an expression vector comprising the polynucleotide molecule. In some embodiments, a host cell comprises a polynucleotide molecule.

### IV. METHODS OF USE AND PHARMACEUTICAL COMPOSITIONS

The BMPR-binding agents (e.g., polypeptides and/or antibodies) of the invention are useful in a variety of applications including, but not limited to, therapeutic treatment methods, such as the treatment of cancer. In certain embodiments, the agents are useful for modulating BMP pathway activity, stimulating and/or increasing BMP pathway activity, stimulating or increasing BMP pathway signaling, increasing and/or enhancing interactions between BMPR type I and II receptors or a combination thereof. In some embodiments, the BMPR-binding agents are useful in inhibiting tumor growth, reducing tumor volume, reducing the tumorigenicity of a tumor, reducing the frequency of cancer stem cells in a tumor, inducing death of tumor cells, inducing differentiation of tumor cells, inhibiting angiogenesis, and/or interfering with angiogenesis. The methods of use may be in vitro, ex vivo, or in vivo methods. In some embodiments, the BMPR-binding agent (e.g., polypeptide and/or antibody) is an agonist of BMPR activation. In certain embodiments, the BMPR-binding agent is an agonist of BMPR1A BMPR1B. In some embodiments, the BMPR-binding agent is an agonist of BMPR2, ACVR2A and/or ACVR2B. In certain embodiments, the BMPR-binding agent is an agonist of the BMP signaling pathway. In certain embodiments, the BMPR-binding agent is an agonist of BMP pathway activation.

In certain embodiments, BMPR-binding agents are used in the treatment of a disease associated with the BMP pathway and/or activation and signaling of the BMP pathway. In particular embodiments, the disease is a disease associated with a BMP signaling pathway. In some embodiments, the disease is a tumor. In some embodiments, the disease is cancer. In some embodiments, tumor growth is associated with an inhibition of a BMP signaling pathway. In some embodiments, tumor growth is associated with an inhibition of BMPR activation. In certain embodiments, tumor growth is associated with an over-expression of a BMP. In certain embodiments, tumor growth is associated with increased activity of the BMP pathway.

The present invention further provides methods for inhibiting tumor growth using the BMPR-binding agents described herein. In certain embodiments, the method of inhibiting tumor growth comprises contacting tumor cells with a BMPR-binding agent (e.g., an antibody) in vitro. For example, an immortalized cell line or a cancer cell line that expresses a BMPR on the cell surface is cultured in medium to which is added the antibody or other agent to inhibit tumor cell growth. In some embodiments, tumor cells are isolated from a patient sample such as, for example, a tissue biopsy, pleural effusion, or blood sample and cultured in medium to which is added a BMPR-binding agent to inhibit tumor growth.

In some embodiments, the method of inhibiting tumor growth comprises contacting the tumor or tumor cells with a BMPR-binding agent (e.g., an antibody) in vivo. In certain embodiments, contacting a tumor or tumor cells with a BMPR-binding agent is undertaken in an animal model. For example, BMPR-binding agents are administered to immunocompromised mice (e.g., NOD/SCID mice) that have xenograft tumors expressing at least one BMPR. After administration of BMPR-binding agents, the mice are observed for inhibition of tumor growth. In some embodiments, cancer stem cells are isolated from a patient sample such as, for example, a tissue biopsy, pleural effusion, or blood sample and injected into immunocompromised mice that are then administered a BMPR-binding agent to inhibit tumor growth. In some embodiments, the BMPR-binding agent is administered at the same time or shortly after introduction of tumorigenic cells (CSCs) into the animal to prevent tumor growth. In some embodiments, the BMPR-binding agent is administered as a therapeutic after the tumorigenic cells have grown to a specified size.

In certain embodiments, the method of inhibiting tumor growth comprises administering to a subject a therapeutically effective amount of a BMPR-binding agent. In certain embodiments, the subject is a human. In certain embodiments, the subject has a tumor. In certain embodiments, the subject has had a tumor removed. In some embodiments, the BMPR-binding agent is an antibody. In some embodiments, the BMPR-binding agent is antibody 5M107. In some embodiments, the BMPR-binding agent comprises the heavy chain variable region of antibody 5M107. In some embodiments, the BMPR-binding agent is a bispecific antibody comprising the heavy chain variable region of antibody 5M107. In some embodiments, the BMPR-binding agent is a bispecific antibody comprising the heavy chain variable region CDRs of 5M107. In some embodiments, the BMPR-binding agent is a bispecific antibody which specifically binds BMPR1A and BMPR2. In some embodiments, the BMPR-binding agent is a bispecific antibody which specifically binds BMPR1B and BMPR2. In some embodiments, the BMPR-binding agent is a bispecific antibody which specifically binds BMPR1A and ACVR2A. In some embodiments, the BMPR-binding agent is a bispecific antibody which specifically binds BMPR1B and ACVR2A. In some embodiments, the BMPR-binding agent is a bispecific antibody which specifically binds BMPR1A and ACVR2B. In some embodiments, the BMPR-binding agent is a bispecific antibody which specifically binds BMPR1B and ACVR2B.

In certain embodiments, the tumor expresses at least one BMPR to which the BMPR-binding agent or antibody binds. In certain embodiments, the tumor over-expresses a human BMPR. In certain embodiments, the tumor expresses at least one BMPR (e.g., BMPR1A, BMPR1B, BMPR2, ACVR2A or ACVR2B) with which a BMP interacts. In some embodiments, the BMPR-binding agent binds at least one BMPR and inhibits or reduces growth of the tumor. In some embodiments, the BMPR-binding agent binds at least one BMPR, enhances BMPR type I/type II interactions and inhibits or reduces growth of the tumor. In some embodiments, the BMPR-binding agent binds at least one BMPR, stimulates and/or increases BMPR activation and inhibits or reduces growth of the tumor. In some embodiments, the BMPR-binding agent binds at least one BMPR, and reduces the frequency of cancer stem cells in the tumor.

In certain embodiments, the tumor is a tumor selected from the group consisting of colorectal tumor, pancreatic tumor, lung tumor, ovarian tumor, liver tumor, breast tumor, kidney tumor, prostate tumor, gastrointestinal tumor, melanoma, cervical tumor, bladder tumor, glioblastoma, and head and neck tumor. In certain embodiments, the tumor is a colorectal tumor. In certain embodiments, the tumor is a pancreatic tumor. In certain embodiments, the tumor is a breast tumor. In certain embodiments, the tumor is a prostate tumor. In certain embodiments, the tumor is a lung tumor. In certain embodiments, the tumor is a glioblastoma. In certain embodiments, the subject is a human.

The present invention further provides methods for treating cancer using the BMPR-binding agents described herein. In certain embodiments, the cancer is characterized by cells expressing at least one BMPR to which the BMPR-binding agent (e.g., antibody) binds. In certain embodiments, the cancer over-expresses a human BMPR. In some embodiments, the BMPR-binding agent binds at least one BMPR and inhibits or reduces growth of the cancer. In some embodiments, the BMPR-binding agent binds at least one BMPR, enhances BMPR type I/type II interactions and inhibits or reduces growth of the cancer. In some embodiments, the BMPR-binding agent binds at least one BMPR, stimulates and/or increases BMP pathway activation and inhibits or reduces growth of the cancer. In some embodiments, the BMPR-binding agent binds at least one BMPR, and reduces the frequency of cancer stem cells in the cancer.

The present invention provides for methods of treating cancer comprising administering a therapeutically effective amount of a BMPR-binding agent to a subject (e.g., a subject in need of treatment). In certain embodiments, the subject is a human. In certain embodiments, the subject has a cancerous tumor. In certain embodiments, the subject has had a tumor removed. In some embodiments, the BMPR-binding agent is an antibody. In some embodiments, the BMPR-binding agent is antibody 5M107. In some embodiments, the BMPR-binding agent is a bispecific antibody comprising the heavy chain variable region (with or without the signal sequence) of 5M107. In some embodiments, the BMPR-binding agent is a bispecific antibody comprising the heavy chain variable region CDRs of 5M107. In some embodiments, the BMPR-binding agent is a bispecific antibody which specifically binds BMPR1A and BMPR2. In some embodiments, the BMPR-binding agent is a bispecific antibody which specifically binds BMPR1B and BMPR2. In some embodiments, the BMPR-binding agent is a bispecific antibody which specifically binds BMPR1A and ACVR2A. In some embodiments, the BMPR-binding agent is a bispecific antibody which specifically binds BMPR1B and ACVR2A. In some embodiments, the BMPR-binding agent is a bispecific antibody which specifically binds BMPR1A and ACVR2B. In some embodiments, the BMPR-binding agent is a bispecific antibody which specifically binds BMPR1B and ACVR2B.

In certain embodiments, the cancer is a cancer selected from the group consisting of colorectal cancer, pancreatic cancer, lung cancer, ovarian cancer, liver cancer, breast cancer, kidney cancer, prostate cancer, gastrointestinal cancer, melanoma, cervical cancer, bladder cancer, glioblastoma, and head and neck cancer. In certain embodiments, the cancer is pancreatic cancer. In certain embodiments, the cancer is colorectal cancer. In certain embodiments, the cancer is breast cancer. In certain embodiments, the cancer is prostate cancer. In certain embodiments, the cancer is lung cancer. In certain embodiments, the cancer is a glioblastoma.

The invention also provides a method of stimulating or increasing BMP pathway signaling or BMP pathway activation in a cell comprising contacting the cell with an effective amount of a BMPR-binding agent. In certain embodiments, the cell is a tumor cell. In certain embodiments, the method is an in vivo method wherein the step of contacting the cell with the BMPR-binding agent comprises administering a therapeutically effective amount of the BMPR-binding agent to the subject. In some embodiments, the method is an in vitro or ex vivo method. In certain embodiments, the BMPR-binding agent stimulates or increases BMPR signaling. In some embodiments, the BMPR-binding agent stimulates or increases BMPR activation. In certain embodiments, the BMPR-binding agent stimulates or increases BMP pathway signaling. In some embodiments, the BMPR-binding agent stimulates or increases BMP pathway activation. In certain embodiments, the BMPR-binding agent stimulates or increases a BMPR/BMP interaction. In certain embodiments, the BMPR signaling is signaling by BMPR1A or BMPR1B. In some embodiments, the BMPR-binding agent is an antibody. In some embodiments, the BMPR-binding agent is antibody 5M107.

The invention also provides a method of inhibiting growth of a tumor, comprising contacting the tumor with an effective amount of an agonist of the BMP pathway. In some embodiments, the invention provides a method of inhibiting growth of a tumor in a subject, comprising administering an effective amount of an agonist of the BMP pathway to the subject. In some embodiments, a method of treating cancer in a subject, comprising administering an effective amount of an agonist of the BMP pathway to the subject is provided. In some embodiments, the agonist is a BMPR-binding agent. In some embodiments, the agonist is a BMP molecule. In some embodiments, the agonist is an antibody. In some embodiments, the agonist increases BMP (e.g., BMP4) expression. In some embodiments, the method decreases the frequency of cancer stem cells in the tumor or cancer. In some embodiments, the tumor or cancer expresses BMPR2 or over-expresses BMPR2.

The invention also provides a method of inhibiting BMP pathway signaling or BMP pathway activation in a cell comprising contacting the cell with an effective amount of a BMPR-binding agent. In certain embodiments, the cell is a tumor cell. In certain embodiments, the method is an in vivo method wherein the step of contacting the cell with the BMPR-binding agent comprises administering a therapeutically effective amount of the BMPR-binding agent to the subject. In some embodiments, the method is an in vitro or ex vivo method. In certain embodiments, the BMPR-binding agent inhibits BMPR signaling. In some embodiments, the BMPR-binding agent inhibits BMPR activation. In certain embodiments, the BMPR-binding agent interferes with a BMPR/BMP interaction. In certain embodiments, the BMPR signaling is signaling by BMPR1A or BMPR1B. In some embodiments, the BMPR-binding agent is an antibody. In some embodiments, the BMPR-binding agent is antibody 5M107.

In addition, the invention provides a method of reducing the tumorigenicity of a tumor in a subject, comprising administering a therapeutically effective amount of a BMPR-binding agent to the subject. In certain embodiments, the tumor comprises cancer stem cells. In certain embodiments, the frequency of cancer stem cells in the tumor is reduced by administration of the BMPR-binding agent. The invention also provides a method of reducing the frequency of cancer stem cells in a tumor, comprising contacting the tumor with an effective amount of a BMPR-binding agent (e.g., an anti-BMPR1A antibody). In some embodiments, the BMPR-binding agent is antibody 5M107. In some embodiments, the BMPR-binding agent is a bispecific antibody comprising the heavy chain variable region CDRs of antibody 5M107.

The invention also provides a method of treating a disease or disorder in a subject, wherein the disease or disorder is characterized by an increased level of stem cells and/or progenitor cells. In some embodiments, the treatment methods comprise administering a therapeutically effective amount of the BMPR-binding agent, polypeptide, or antibody to the subject.

The invention also provides methods of inhibiting tumor growth in a subject, comprising (a) determining if the tumor expresses BMPR2 or over-expresses BMPR2, and (b) administering to the subject a therapeutically effective amount of an agonist of the BMP pathway to the subject. Also provided are methods of treating cancer in a subject, comprising: (a) selecting a subject for treatment based, at least in part, on the subject having a cancer that expresses BMPR2 or over-expresses BMPR2, and (b) administering to the subject a therapeutically effective amount of an agonist of the BMP pathway to the subject. In some embodiments, the agonist of the BMP pathway is a BMPR-binding agent. In some embodiments, the agonist of the BMP pathway is a BMP molecule. In some embodiments, the agonist of the BMP pathway is an antibody.

The present invention further provides pharmaceutical compositions comprising one or more of the BMPR-binding agents described herein. In certain embodiments, the pharmaceutical compositions further comprise a pharmaceutically acceptable vehicle. These pharmaceutical compositions find use in inhibiting tumor growth and treating cancer in a subject (e.g., a human patient).

In certain embodiments, formulations are prepared for storage and use by combining a purified antibody or agent of the present invention with a pharmaceutically acceptable vehicle (e.g., a carrier or excipient). Suitable pharmaceutically acceptable vehicles include, but are not limited to, nontoxic buffers such as phosphate, citrate, and other organic acids; salts such as sodium chloride; antioxidants including ascorbic acid and methionine; preservatives such as octadecyldimethylbenzyl ammonium chloride, hexamethonium chloride, benzalkonium chloride, benzethonium chloride, phenol, butyl or benzyl alcohol, alkyl parabens, such as methyl or propyl paraben, catechol, resorcinol, cyclohexanol, 3-pentanol, and m-cresol; low molecular weight polypeptides (e.g., less than about 10 amino acid residues); proteins such as serum albumin, gelatin, or immunoglobulins; hydrophilic polymers such as polyvinylpyrrolidone; amino acids such as glycine, glutamine, asparagine, histidine, arginine, or lysine; carbohydrates such as monosaccharides, disaccharides, glucose, mannose, or dextrins; chelating agents such as EDTA; sugars such as sucrose, mannitol, trehalose or sorbitol; salt-forming counter-ions such as sodium; metal complexes such as Zn-protein complexes; and non-ionic surfactants such as TWEEN or polyethylene glycol (PEG). (Remington: The Science and Practice of Pharmacy, 21st Edition, University of the Sciences in Philadelphia, 2005).

The pharmaceutical compositions of the present invention can be administered in any number of ways for either local or systemic treatment. Administration can be topical by epidermal or transdermal patches, ointments, lotions, creams, gels, drops, suppositories, sprays, liquids and powders; pulmonary by inhalation or insufflation of powders or aerosols, including by nebulizer, intratracheal, and intranasal; oral; or parenteral including intravenous, intraarterial, intratumoral, subcutaneous, intraperitoneal, intramuscular (e.g., injection or infusion), or intracranial (e.g., intrathecal or intraventricular).

The therapeutic formulation can be in unit dosage form. Such formulations include tablets, pills, capsules, powders, granules, solutions or suspensions in water or non-aqueous media, or suppositories. In solid compositions such as tablets the principal active ingredient is mixed with a pharmaceutical carrier. Conventional tableting ingredients include corn starch, lactose, sucrose, sorbitol, talc, stearic acid, magnesium stearate, dicalcium phosphate or gums, and diluents (e.g., water). These can be used to form a solid preformulation composition containing a homogeneous mixture of a compound of the present invention, or a non-toxic pharmaceutically acceptable salt thereof. The solid preformulation composition is then subdivided into unit dosage forms of a type described above. The tablets, pills, etc. of the formulation or composition can be coated or otherwise compounded to provide a dosage form affording the advantage of prolonged action. For example, the tablet or pill can comprise an inner composition covered by an outer component. Furthermore, the two components can be separated by an enteric layer that serves to resist disintegration and permits the inner component to pass intact through the stomach or to be delayed in release. A variety of materials can be used for such enteric layers or coatings, such materials include a number of polymeric acids and mixtures of polymeric acids with such materials as shellac, cetyl alcohol and cellulose acetate.

The BMPR-binding agents or antibodies described herein can also be entrapped in microcapsules. Such microcapsules are prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nanoparticles and nanocapsules) or in macroemulsions as described in Remington: The Science and Practice of Pharmacy, 21st Edition, University of the Sciences in Philadelphia, 2005.

In certain embodiments, pharmaceutical formulations include BMPR-binding agents (e.g., an antibody) of the present invention complexed with liposomes. Methods to produce liposomes are known to those of skill in the art. For example, some liposomes can be generated by reverse phase evaporation with a lipid composition comprising phosphatidylcholine, cholesterol, and PEG-derivatized phosphatidylethanolamine (PEG-PE). Liposomes can be extruded through filters of defined pore size to yield liposomes with the desired diameter.

In certain embodiments, sustained-release preparations can be produced. Suitable examples of sustained-release preparations include semi-permeable matrices of solid hydrophobic polymers containing the BMPR-binding agent (e.g., an antibody), where the matrices are in the form of shaped articles (e.g., films or microcapsules). Examples of sustained-release matrices include polyesters, hydrogels such as poly(2-hydroxyethyl-methacrylate) or poly(vinyl alcohol), polylactides, copolymers of L-glutamic acid and 7 ethyl-L-glutamate, non-degradable ethylene-vinyl acetate, degradable lactic acid-glycolic acid copolymers such as the LUPRON DEPOT™ (injectable microspheres composed of lactic acid-glycolic acid copolymer and leuprolide acetate), sucrose acetate isobutyrate, and poly-D-(−)-3-hydroxybutyric acid.

In certain embodiments, in addition to administering a BMPR-binding agent, the method or treatment further comprises administering at least one additional therapeutic agent. An additional therapeutic agent can be administered prior to, concurrently with, and/or subsequently to, administration of the BMPR-binding agent. Pharmaceutical compositions comprising the BMPR-binding agent and the additional therapeutic agent(s) are also provided. In some embodiments, the at least one additional therapeutic agent comprises 1, 2, 3, or more additional therapeutic agents.

Combination therapy with at least two therapeutic agents often uses agents that work by different mechanisms of action, although this is not required. Combination therapy using agents with different mechanisms of action may result in additive or synergetic effects. Combination therapy may allow for a lower dose of each agent than is used in monotherapy, thereby reducing toxic side effects. Combination therapy may decrease the likelihood that resistant cancer cells will develop. In some embodiments, combination therapy comprises a therapeutic agent that affects (e.g., inhibits or kills) non-tumorigenic cells and a therapeutic agent that affects (e.g., inhibits or kills) tumorigenic CSCs.

It will be appreciated that the combination of a BMPR-binding agent and an additional therapeutic agent may be administered in any order or concurrently. In some embodiments, the BMPR-binding agents will be administered to patients that have previously undergone treatment with a second therapeutic agent. In certain other embodiments, the BMPR-binding agent and a second therapeutic agent will be administered substantially simultaneously or concurrently. For example, a subject may be given the BMPR-binding agent (e.g., an antibody) while undergoing a course of treatment with a second therapeutic agent (e.g., chemotherapy). In certain embodiments, the BMPR-binding agent will be administered within 1 year of the treatment with a second therapeutic agent. In certain alternative embodiments, the BMPR binding agent will be administered within 10, 8, 6, 4, or 2 months of any treatment with a second therapeutic agent. In certain other embodiments, the BMPR-binding agent will be administered within 4, 3, 2, or 1 weeks of any treatment with a second therapeutic agent. In some embodiments, the BMPR-binding agent will be administered within 5, 4, 3, 2, or 1 days of any treatment with a second therapeutic agent. It will further be appreciated that the two (or more) agents or treatments may be administered to the subject within a matter of hours or minutes (i.e., substantially simultaneously).

Useful classes of therapeutic agents include, for example, antitubulin agents, auristatins, DNA minor groove binders, DNA replication inhibitors, alkylating agents (e.g., platinum complexes such as cisplatin, mono(platinum), bis(platinum) and tri-nuclear platinum complexes and carboplatin), anthracyclines, antibiotics, antifolates, antimetabolites, chemotherapy sensitizers, duocarmycins, etoposides, fluorinated pyrimidines, ionophores, lexitropsins, nitrosoureas, platinols, purine antimetabolites, puromycins, radiation sensitizers, steroids, taxanes, topoisomerase inhibitors, vinca alkaloids, or the like. In certain embodiments, the second therapeutic agent is an antimetabolite, an antimitotic, a topoisomerase inhibitor, or an angiogenesis inhibitor.

Therapeutic agents that may be administered in combination with the BMPR-binding agents include chemotherapeutic agents. Thus, in some embodiments, the method or treatment involves the combined administration of a BMPR-binding agent or antibody of the present invention and a chemotherapeutic agent or cocktail of multiple different chemotherapeutic agents. Treatment with an antibody can occur prior to, concurrently with, or subsequent to administration of chemotherapies. Combined administration can include co-administration, either in a single pharmaceutical formulation or using separate formulations, or consecutive administration in either order but generally within a time period such that all active agents can exert their biological activities simultaneously. Preparation and dosing schedules for such chemotherapeutic agents can be used according to manufacturers' instructions or as determined empirically by the skilled practitioner. Preparation and dosing schedules for such chemotherapy are also described in Chemotherapy Service Ed., M. C. Perry, Williams & Wilkins, Baltimore, Md. (1992).

Chemotherapeutic agents useful in the instant invention include, but are not limited to, alkylating agents such as thiotepa and cyclosphosphamide (CYTOXAN); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamime; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tabercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytosine arabinoside, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenishers such as folinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK; razoxane; sizofuran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C); taxoids, e.g. paclitaxel (TAXOL) and docetaxel (TAXOTERE); chlorambucil; gemcitabine; 6-thioguanine; mercaptopurine; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT11; topoisomerase inhibitor RFS 2000; difluoromethylornithine (DMFO); retinoic acid; esperamicins; capecitabine; and pharmaceutically acceptable salts, acids or derivatives of any of the above. Chemotherapeutic agents also include anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY117018, onapristone, and toremifene (Fareston); and anti-androgens such as flutamide, nilutamide, bicalutamide, leuprolide, and goserelin; and pharmaceutically acceptable salts, acids or derivatives of any of the above.

In certain embodiments, the chemotherapeutic agent is a topoisomerase inhibitor. Topoisomerase inhibitors are chemotherapy agents that interfere with the action of a topoisomerase enzyme (e.g., topoisomerase I or II). Topoisomerase inhibitors include, but are not limited to, doxorubicin HCL, daunorubicin citrate, mitoxantrone HCl, actinomycin D, etoposide, topotecan HCl, teniposide (VM-26), and irinotecan, as well as pharmaceutically acceptable salts, acids, or derivatives of any of these. In certain embodiments, the second therapeutic agent is irinotecan.

In certain embodiments, the chemotherapeutic agent is an anti-metabolite. An anti-metabolite is a chemical with a structure that is similar to a metabolite required for normal biochemical reactions, yet different enough to interfere with one or more normal functions of cells, such as cell division. Anti-metabolites include, but are not limited to, gemcitabine, fluorouracil, capecitabine, methotrexate sodium, ralitrexed, pemetrexed, tegafur, cytosine arabinoside, THIOGUANINE, 5-azacytidine, 6-mercaptopurine, azathioprine, 6-thioguanine, pentostatin, fludarabine phosphate, and cladribine, as well as pharmaceutically acceptable salts, acids, or derivatives of any of these. In certain embodiments, the second therapeutic agent is gemcitabine.

In certain embodiments, the chemotherapeutic agent is an antimitotic agent, including, but not limited to, agents that bind tubulin. In some embodiments, the agent is a taxane. In certain embodiments, the agent is paclitaxel or docetaxel, or a pharmaceutically acceptable salt, acid, or derivative of paclitaxel or docetaxel. In certain embodiments, the agent is paclitaxel (TAXOL), docetaxel (TAXOTERE), albumin-bound paclitaxel (ABRAXANE), DHA-paclitaxel, or PG-paclitaxel. In certain alternative embodiments, the antimitotic agent comprises a vinca alkaloid, such as vincristine, binblastine, vinorelbine, or vindesine, or pharmaceutically acceptable salts, acids, or derivatives thereof. In some embodiments, the antimitotic agent is an inhibitor of kinesin Eg5 or an inhibitor of a mitotic kinase such as Aurora A or Plk1. In certain embodiments, where the chemotherapeutic agent administered in combination with the BMPR-binding agent is an anti-mitotic agent, the cancer or tumor being treated is breast cancer or a breast tumor.

In certain embodiments, the treatment involves the combined administration of a BMPR-binding agent (e.g. an antibody) of the present invention and radiation therapy. Treatment with the BMPR-binding agent can occur prior to, concurrently with, or subsequent to administration of radiation therapy. Dosing schedules for such radiation therapy can be determined by the skilled medical practitioner.

In some embodiments, a second therapeutic agent comprises an antibody. Thus, treatment can involve the combined administration of a BMPR-binding agent (e.g. an antibody) of the present invention with other antibodies against additional tumor-associated antigens including, but not limited to, antibodies that bind to EGFR, ErbB2, HER2, DLL4, Notch and/or VEGF. Exemplary, anti-DLL4 antibodies, are described, for example, in U.S. Patent Application Pub. No. 2008/0187532. Additional anti-DLL4 antibodies are described in, e.g., International Patent Pub. Nos. WO 2008/091222 and WO 2008/0793326, and U.S. Patent Application Pub. Nos. 2008/0014196; 2008/0175847; 2008/0181899; and 2008/0107648. Exemplary anti-Notch antibodies are described, for example, in U.S. Patent Application Pub. No. 2008/0131434. In certain embodiments, a second therapeutic agent is an antibody that is an angiogenesis inhibitor (e.g., an anti-VEGF antibody). In certain embodiments, a second therapeutic agent is bevacizumab (AVASTIN), trastuzumab (HERCEPTIN), panitumumab (VECTIBIX), or cetuximab (ERBITUX). Combined administration can include co-administration, either in a single pharmaceutical formulation or using separate formulations, or consecutive administration in either order but generally within a time period such that all active agents can exert their biological activities simultaneously.

Furthermore, treatment with the BMPR-binding agents described herein can include combination treatment with one or more cytokines (e.g., lymphokines, interleukins, tumor necrosis factors, and/or growth factors) or can be accompanied by surgical removal of tumors, cancer cells or any other therapy deemed necessary by a treating physician.

For the treatment of the disease, the appropriate dosage of an BMPR-binding agent (e.g., an antibody) of the present invention depends on the type of disease to be treated, the severity and course of the disease, the responsiveness of the disease, whether the BMPR-binding agent or antibody is administered for therapeutic or preventative purposes, previous therapy, the patient's clinical history, and so on, all at the discretion of the treating physician. The BMPR-binding agent or antibody can be administered one time or over a series of treatments lasting from several days to several months, or until a cure is effected or a diminution of the disease state is achieved (e.g., reduction in tumor size). Optimal dosing schedules can be calculated from measurements of drug accumulation in the body of the patient and will vary depending on the relative potency of an individual antibody or agent. The administering physician can easily determine optimum dosages, dosing methodologies and repetition rates. In certain embodiments, dosage is from 0.01 μg to 100 mg per kg of body weight, and can be given once or more daily, weekly, monthly or yearly. In certain embodiments, the antibody or other BMPR-binding agent is given once every two weeks or once every three weeks. In certain embodiments, the dosage of the antibody or other BMPR-binding agent is from about 0.1 mg to about 20 mg per kg of body weight. The treating physician can estimate repetition rates for dosing based on measured residence times and concentrations of the drug in bodily fluids or tissues.

### EXAMPLES

### Example 1

### Over-Expression of BMP4 in Primary Human Tumors

Lentivial expression of BMP4 in tumor xenografts was used to evaluate the impact of BMP signaling activation on tumor engraftment and tumor growth. BMP4 was over-expressed in a variety of primary human tumors using a lentiviral delivery system. Tumor take and tumor growth from tumors over-expressing BMP4 were evaluated in mouse xenograft models. The primary human tumors used were breast tumors UM-T3 and UM-PE13, colon tumors UM-C6, UM-C8, OMP-C11, OMP-C17 and OMP-C18, pancreatic tumor OMP-PN8 and melanoma tumor OMP-M3.

An HIV-1-based lentiviral vector containing a constitutive BMP2/BMP4 fusion gene-IRES-GFP expression cassette with a CMV promoter (LentiBMP4-GFP) was used to transduce freshly isolated tumor cells ex vivo. The lentiviral vector was constructed as described in Peng et al., 2001, Mol. Therapy. 4:95-104. Single cell suspensions were obtained from minimally passaged xenografts by mechanical dissociation and enzymatic digestion with collagenase III and DNaseI for 2 hours at 37° C. The cell suspensions were incubated with biotinylated anti-mouse H-2Kd and anti-mouse CD45 antibodies on ice for 30 minutes followed by addition of streptavidin-labeled magnetic beads (MagnaBind Streptavidin Beads, ThermoScientific, Rockford, Ill.). Mouse cells bound with biotinylated antibodies were removed with the aid of a magnet. The remaining human tumor cells were infected with 2.5 transducing units per cell of the LentiBMP4-GFP vector or a control vector that expressed only GFP (LentiGFP). The infection medium was replaced with fresh culture medium after one day. The culture medium contained 72% low glucose DMEM, 24% F-12, 1×B-27 supplement, 1 ug/ml hydrocortisone, 1×ITS-X (insulin-transferrin-selenium-X), 1× antibiotics, 20 ng/ml rhEGF, 20 ng/ml bFGF, and heparin. For colon tumor cells, the culture medium was additionally supplemented with hLIF. After 3 days in culture, the transduced cells were sorted by GFP expression using a FACSAria cell sorter (BD Biosciences, San Jose, Calif.). 200-1000 GFP-positive cells were re-suspended in 50 ul HBSS supplemented with 2% FBS and HEPES plus 50 μl Matrigel™. Cells were injected subcutaneously in the flanks of NOD/SCID mice. 10 mice per group each received 100 μl of cell suspension. Tumor take was monitored weekly and engrafted tumors were measured weekly. The experiment was terminated when the fastest growing tumor reached 1500 mm³ in size.

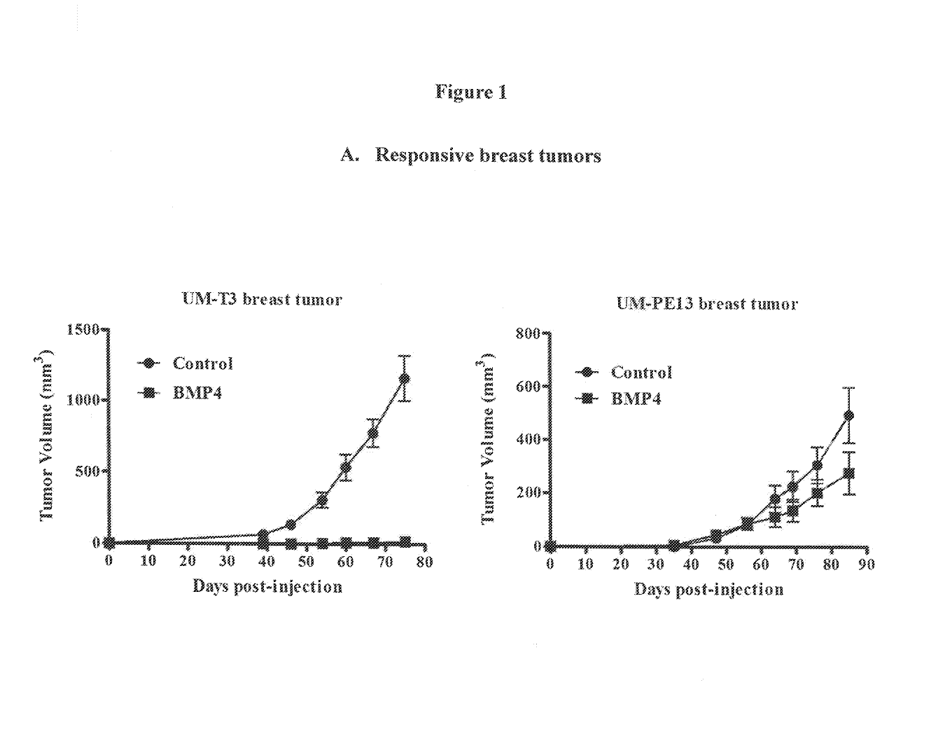

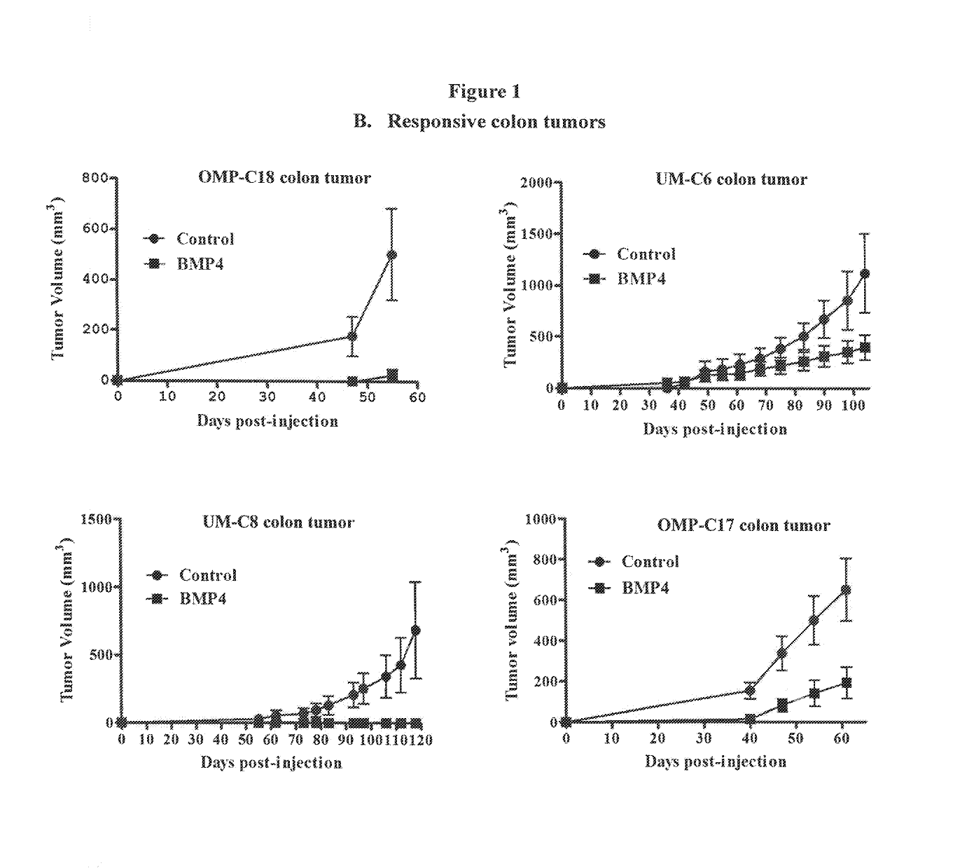

As shown in FIG. 1A, growth of breast tumors UM-T3 and UM-PE13 transduced with LentiBMP4-GFP was inhibited as compared to tumors transduced with control vector LentiGFP. As shown in FIG. 1B, growth of four of the colon tumors, OMP-C6, UM-C8, OMP-C17 and OMP-C18, transduced with LentiBMP4-GFP was inhibited as compared to tumors transduced with control vector. As shown in FIG. 1C, growth of the remaining tumors, colon tumor OMP-C11, pancreatic tumor OMP-PN8 and melanoma tumor OMP-M3 transduced with LentiBMP4-GFP was not inhibited. TaqMan assays were used to demonstrate that BMP4 was expressed in all tumors (data not shown). These data suggested that activation of the BMP pathway could have an inhibitory effect on in vivo tumor growth.

### Example 2

### BMP4 Treatment of Colon Tumor OMP-C18

Single cell suspensions of colon tumor OMP-C18 were obtained from minimally passaged xenografts by mechanical dissociation and enzymatic digestion with collagenase III and DNaseI for 2 hours at 37° C. Approximately 50,000 cells were injected subcutaneously in the flanks of NOD-SCID mice. On day 29 when the tumors reached an average size of 150 mm³, the mice were randomized, and placed in groups of 10. An adenoviral vector was used to deliver a CMV-BMP4 cassette (Ad-BMP4) to the mice and to express BMP4. An adenoviral vector containing a Fc cassette (Ad-Fc) was used as a negative control vector. 10⁹ pfus of the appropriate vector were administered to each mouse through a single tail vein injection. Tumor growth was monitored over the next 11 days and tumors were measured weekly with a digital caliper. BMP4 was detected in mouse sera after adenoviral delivery by Western blot analyses and the amount of BMP4 was found to remain stable for the duration of the experiment (data not shown).

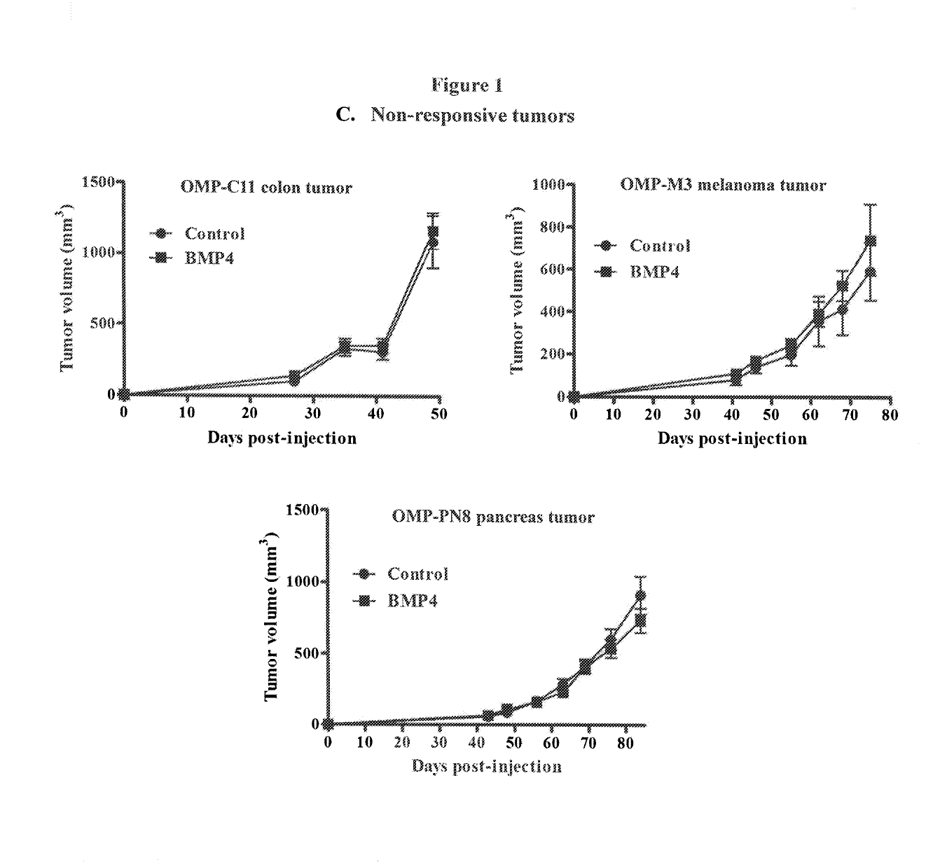

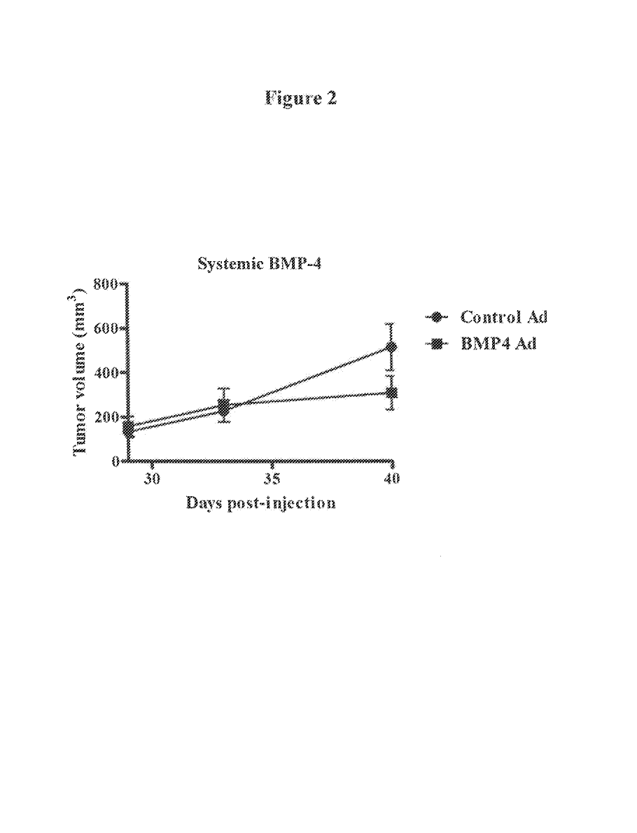

As shown in FIG. 2, growth of colon tumor OMP-C18 was inhibited by adenovirus delivered BMP4. Eleven days after a single virus injection, BMP4-treated tumors were 40% smaller (on average) than the control-treated tumors. The experiment was not extended beyond 11 days post-injection due to the toxic effect of systemic expression of BMP4 in the mice. Control mice injected with the Ad-Fc vector did not show any toxic effects.

### Example 3

### FACS Analysis of BMP4-Treated Colon Tumor OMP-C18

OMP-C18 colon tumors from Example 2 were harvested and analyzed by FACS for the expression of cancer stem cell markers ESA, CD44 and CD166. Single cell suspensions were obtained from the BMP4-treated and the control-treated tumors by mechanical dissociation and enzymatic digestion with collagenase III and DNaseI for 2 hours at 37° C. Approximately 1×10⁶ cells of each tumor were incubated in 100 μl of staining solution with a mixture of the following antibodies: 1 μl biotinylated anti-mouse H-2Kd, 0.5 μl biotinylated anti-mouse CD45, 20 μl phycoerythrin (PE)-conjugated anti-human CD166, 2 μl allophycocyanin (APC)-conjugated anti-human ESA and 2 μl PE-Cy7-conjugated anti-human and anti-mouse CD44. A second incubation with 0.5 μl PE-Cy5.5-conjugated streptavidin was performed to detect the mouse cells bound with biotinylated antibodies. DAPI was added to the final solution to allow for detection of dead cells. The cells were analyzed on a CANTOII FACS instrument (BD Biosciences, San Jose, Calif.) and the data was processed using DIVA software.

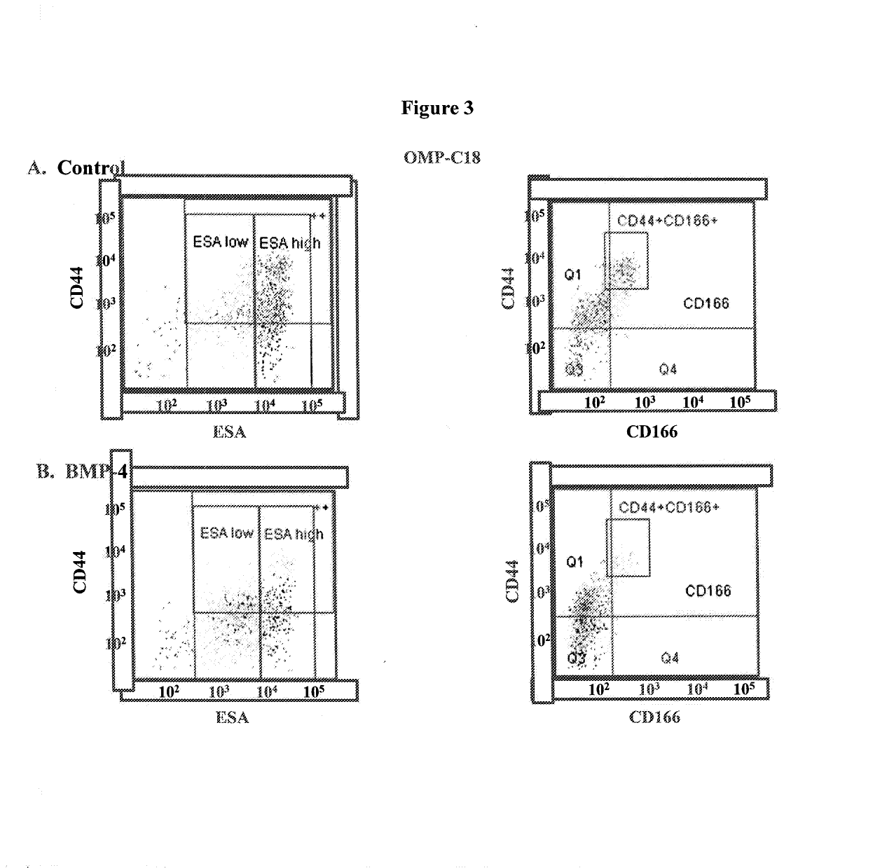

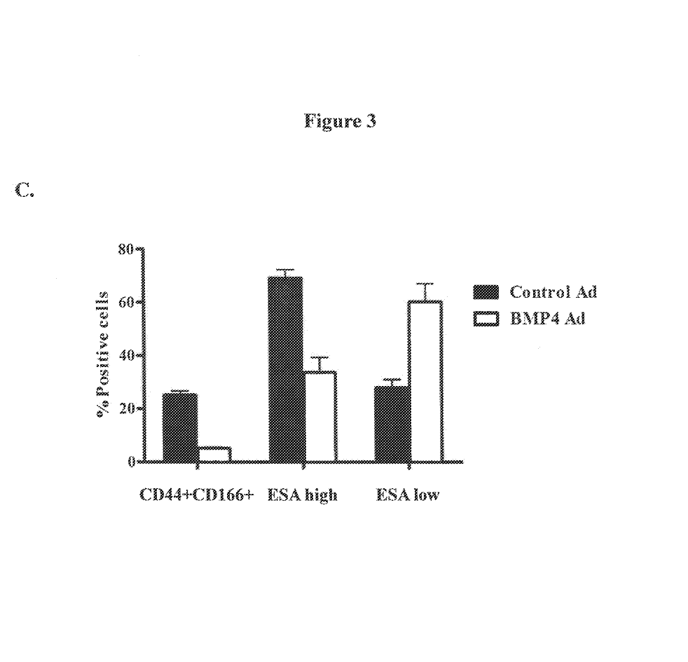

As shown in FIG. 3A, the mean fluorescence intensity of the ESA^{high} signal was reduced in the BMP4-treated tumor cells as compared to the control-treated tumor cells. The mean fluorescence intensity of the CD44+CD166+ signal was also reduced in the BMP-4-treated tumor cells as compared to the control-treated tumor cells (FIG. 3B). Analysis of the FACS data revealed that there was approximately a 50% reduction in the ESA^{high} cells in the BMP4-treated tumor cells. The ESA^{high} cells appeared to shift to an ESA^{low} phenotype. Analysis of the FACS data also revealed that there was approximately a 5-fold reduction in the amount of CD44+CD166+ cells in the BMP4-treated tumor cells. The percentage of marker-positive cells obtained for each tumor was averaged for the BMP4-treated group and the control-treated group and a clear reduction in CD44+CD166+ cells in the BMP4-treated group was observed. A reduction in the percentage of ESA^{high} positive cells with a corresponding increase in the percentage of ESA^{low} positive cells in the BMP4-treated group was also observed (FIG. 3C). These results suggest a decrease in the number or frequency of cancer stem cells in the BMP4-treated tumors.

### Example 4

### Limiting Dilution Analysis of BMP4-Treated Colon Tumor OMP-C18 Cells

Control and treated tumors from the OMP-C18 xenograft study described above (Example 2) were harvested at the end of the study. Three OMP-C18 colon tumors from the control-treated group and three tumors from the BMP4-treated group were pooled and analyzed by limiting dilution analysis. As previously described, single cell suspensions were obtained by mechanical dissociation and enzymatic digestion with collagenase III and DNaseI for 2 hours at 37° C. The cell suspensions were incubated with biotinylated mouse antibodies (anti-H-2Kd and anti-CD45) on ice for 30 minutes followed by addition of streptavidin-labeled magnetic beads (MagnaBind Streptavidin Beads, ThermoScientific, Rockford, Ill.). Mouse cells were removed with the aid of a magnet.

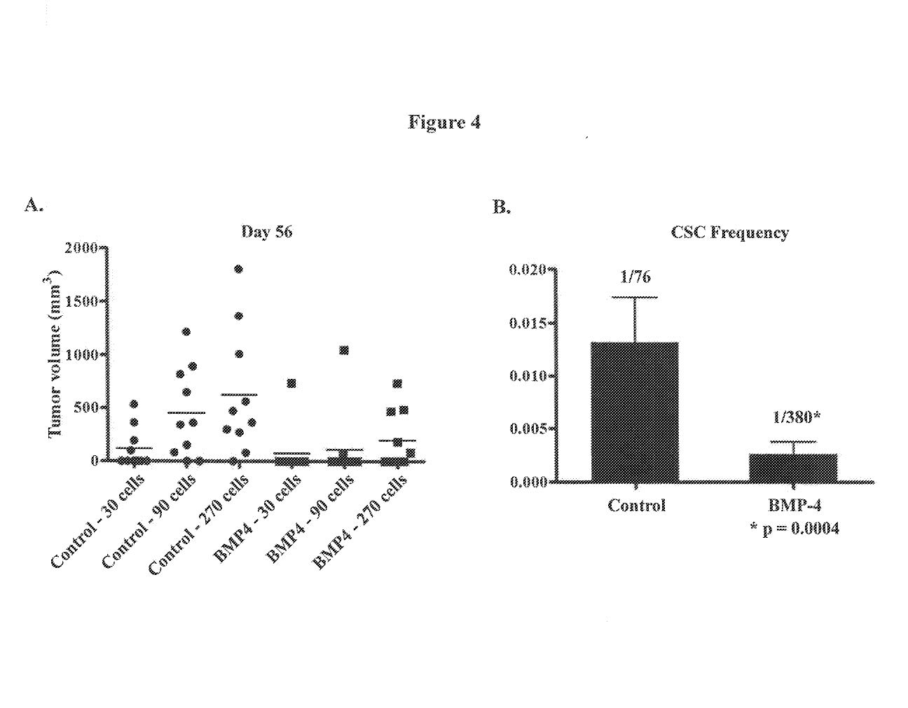

For the limiting dilution assay (LDA), the human tumor cells in the suspension were harvested, counted, and a series of cell doses (30, 90, and 270 cells) were injected subcutaneously in the flanks of a series of NOD/SCID mice (10 mice per cell dose per treatment group). Tumor take and tumor volume were assessed after 56 days. As shown in FIG. 4A, fewer tumors were observed in the animals injected with cells from the BMP4-treated tumors at all three doses as compared to the control animals injected with cells from the control-treated tumors at equivalent doses. In addition, the average tumor volume was smaller in the animals injected with cells from BMP4-treated tumors as compared to animals injected with cells from control-treated tumors. The percentage of mice with detectable tumors was determined in all groups injected with cells from BMP4-treated tumors and compared to percentage of mice with detectable tumors in all groups injected with cells from control-treated tumors. For example, the number of mice injected with 90 control-treated tumor cells that had detectable tumors was determined and compared to the number of mice injected with 90 BMP4-treated tumor cells that had detectable tumors. The cancer stem cell frequency was calculated using L-Calc™ software (StemCell Technologies Inc., Vancouver, BC). Briefly, based on Poisson statistics, exactly one cancer stem cell exists among the known number of injected cells if 37% of the animals fail to develop tumors. As shown in FIG. 4B, the cancer stem cell frequency of the BMP4-treated tumor cells was 1/380 while the cancer stem frequency of the control-treated tumor cells was 1/76. Thus, the CSC frequency in the BMP4-treated tumors was decreased by greater than 5-fold (p=0.0004) as compared to control-treated tumors.

### Example 5

### Dose Response of BMP4 Treatment in Colon Tumor OMP-C18

Systemic administration of a single high dose of BMP4 induced toxicity in mice along with anti-tumor activity (see Example 2). Mice lost weight and presented with mild GI tract symptoms. Six doses of AdBMP4 were used to set up a dose response experiment to identify the highest active dose and to identify a range of AdBMP4 doses which demonstrated anti-tumor activity without systemic toxicity.

Single cell suspensions of colon tumor OMP-C18 were obtained from minimally passaged xenografts by mechanical dissociation and enzymatic digestion with collagenase III and DNaseI for 2 hours at 37° C. Approximately 50,000 cells were injected subcutaneously in the flanks of NOD-SCID mice. When the tumors reached an average size of 150 mm³ the mice were randomized, and placed in groups of 5. As described above in Example 2, an adenoviral vector was used to deliver a CMV-BMP4 cassette (AdBMP4) to the mice and express BMP4. 3.5×10⁸, 1.75×10⁸, 8.75×10⁷, 4.38×10⁷, 2.19×10⁷ and 1.09×10⁷ pfu of AdBMP4 were administered to each mouse through a single tail vein injection. AdFc was used as a negative control vector at 3.5×10⁸ pfu. Tumor growth was monitored for 18 days post-injection, and tumors were measured with a digital caliper.

At day 42 the tumors were harvested and analyzed by FACS for the expression of cancer stem markers ESA and CD44. Single cell suspensions were obtained from the BMP4-treated and the control-treated tumors by mechanical dissociation and enzymatic digestion with collagenase III and DNaseI for 2 hours at 37° C. Approximately 1×10⁶ cells of each tumor were incubated in 100 μl of staining solution with a mixture of the following antibodies: 1 μl biotinylated anti-mouse H-2Kd, 0.5 μl biotinylated anti-mouse CD45, 2 μl allophycocyanin (APC)-conjugated anti-human ESA and 2 μl PE-Cy7-conjugated anti-human and anti-mouse CD44. A second incubation with 0.5 μl PE-Cy5.5-conjugated streptavidin was performed to detect the mouse cells bound with biotinylated antibodies. DAPI was added to the final solution to allow for detection of dead cells. The cells were analyzed on a CANTOII FACS instrument (BD Biosciences, San Jose, Calif.) and the data was processed using DIVA software.

The health status of the mice was carefully monitored during the experiment. The mice were weighed once weekly, and an anatomopathology study was performed on a few tissues in which BMP4 is known to exert a morphogenetic activity. Pancreas, muscle and intestine tissues were harvested from all mice at the time of sacrifice and the tissues were fixed in 10% formalin for 24 hours. Additional tissues were harvested from mice injected with the highest dose of AdDMP4. Fixed tissues were embedded in paraffin, 5 μm sections were cut, mounted on slides and hematoxylin and eosin staining was performed. Gross and microscopic anatomies were evaluated by a pathologist.

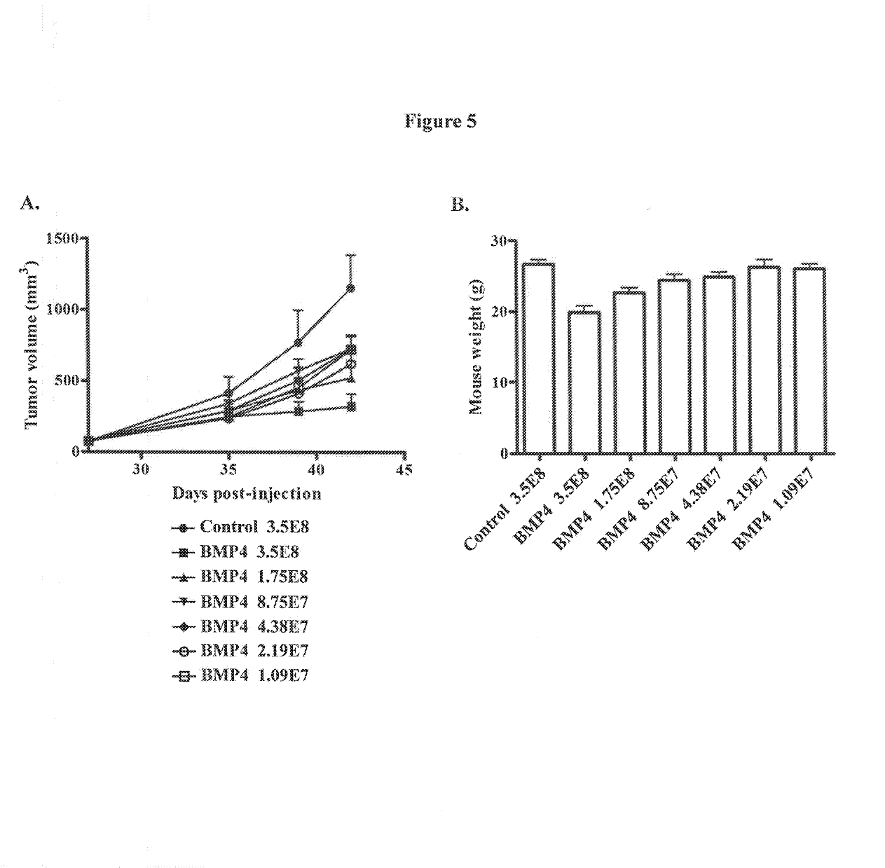

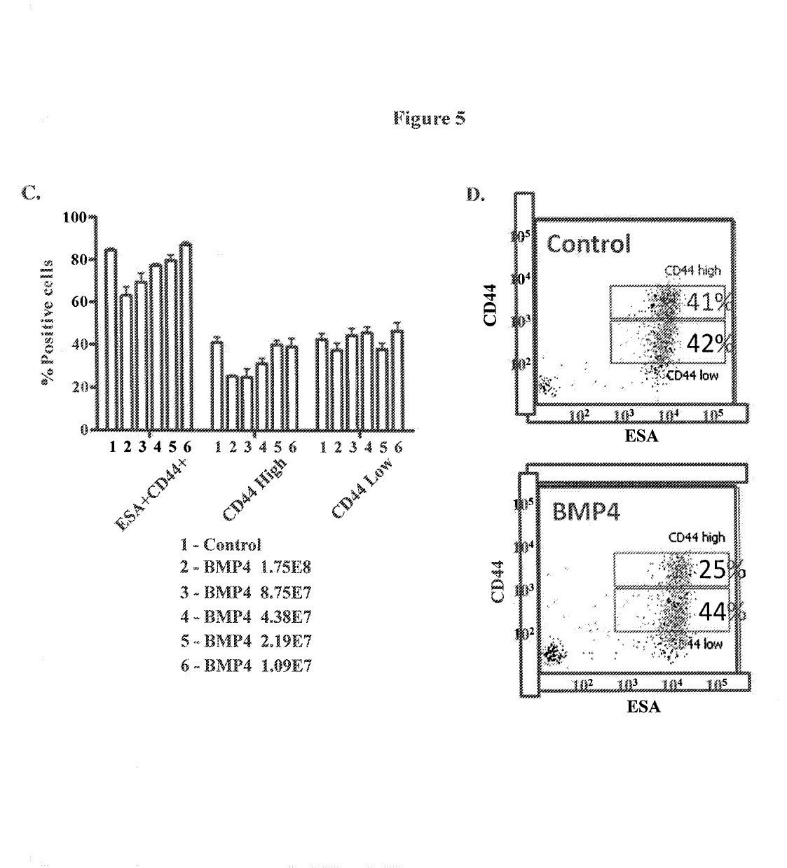

Growth of colon tumor OMP-C18 was inhibited by adenovirus directed BMP4 expression at all virus doses as compared to treatment with the control vector (FIG. 5A). In addition, FACS analysis showed that the 2 to 3 highest virus doses significantly decreased ESA and CD44 expression levels on the BMP4-treated tumor cells (dose 1.75×10⁸ p=0.0004; dose 8.75×10⁷ p=0.009; dose 4.38×10⁷ p=0.0004). The reduction in the number of CD44^{high} cells was not mirrored by an increase in CD44^{low} cells (FIG. 5C). However, the reduction in CD44^{high} cells appeared to correlate with the appearance of double negative cells (FIG. 5D).

The two highest doses of Ad-BMP4, 3.5×10⁸ and 1.75×10⁸ pfus, had a toxic effect on the mice. This was demonstrated by weight loss (FIG. 5B) and also by the early deaths of mice in these two groups. The main pathology seen in the BMP4-treated animals was a mild depletion of the pancreatic zymogen granules in the three highest doses, 3.5×10⁸, 1.75×10⁸, and 8.75×10⁷ (Table 1). This effect was absent from all three lower dose groups, correlating with the weight loss observations. The bone pathology observed in the highest dose animals was not analyzed in lower dose samples, and thus no correlation with BMP4 doses was established. Importantly, the three lowest doses of AdBMP4 inhibited the growth of the colon tumor OMP-C18 relative to the control vector and showed no toxicity, demonstrating that there were effective and safe doses of BMP4 expression in this model.

TABLE 1

| | Days Post | | | |
|---|---|---|---|---|
| Group | Injection of | | | |
| Dose (pfu) | Virus | Animal ID | Tissue(s) | Change Description |
| AdFc | 18 | 202, 218, 229, 246, 253 | Pancreas, muscle, | No Significant Findings |
| 3.5 × 10⁸ | | | intestine | |
| AdBMP4 | 18 | 206, 217, 219, 231, 227 | Pancreas, muscle, | No Significant Findings |
| 1.09 × 10⁷ | | | intestine | |
| AdBMP4 | 18 | 211, 220, 225, 234, 255 | Pancreas, muscle, | No Significant Findings |
| 2.19 × 10⁷ | | | intestine | |
| AdBMP4 | 18 | 223, 226, 232, 249, 252 | Pancreas, muscle, | No Significant Findings |
| 4.38 × 10⁷ | | | intestine | |
| AdBMP4 | 18 | 222, 239, 241, 248, 254 | Pancreas, muscle | Mild to moderate depletion |
| 8.75 × 10⁷ | | | intestine | of pancreatic zymogen |
| | | | | granules (4 of 5 animals) |
| AdBMP4 | 18 | 221, 236 | Pancreas, muscle, | Mild depletion of pancreatic |
| 1.75 × 10⁸ | | | intestine | zymogen granules |
| | | | | (2 of 2 animals) |
| AdBMP4 | 7 | 233, 247 | Pancreas | No Significant Findings |
| 3.50 × 10⁸ | 14 | 210, 216, 224 | Pancreas | Minimal pancreatic |
| | | | | zymogen depletion |
| | | | | (3 of 3 animals) |
| | 7, 14 | 233, 247 (7) | Bone (femur and | Exostosis extending to |
| | | 210, 216, 224 (14) | spinal column) | surrounding muscle |
| | | | | (4 of 5 animals); |
| | | | | 1 poor sample—mild |
| | | | | periosteal |
| | | | | hypertrophy/hyperplasia |
| | | 233, 247 (7) | Small intestine | Minimal focal epithelial |
| | | 210 (14) | | degeneration |
| | | | Brain, kidneys, | No Significant Findings |
| | | | spleen, heart, lungs, | |
| | | | isolated muscle, | |
| | | | intestine | |

### Example 6

### Establishment of a BMP Gene Signature in Colon Tumor

The effect of BMP4 over-expression on colon tumor cell gene expression was tested in colon tumor OMP-C18. Control and treated tumors from the OMP-C18 xenograft study described above (Example 2) were harvested at the end of the study and stored in RNAlater® (Qiagen, Valencia, Calif.). Total RNA was extracted from homogenized whole tumors using Qiagen's RNeasy® mini-prep kit. The global gene expression profiling analysis was performed on Affymetrix Human Genome U133 Plus 2.0 and Mouse Genome 430 2.0 array chips (Affymetrix, Santa Clara, Calif.). Three independent RNA samples of xenograft whole tumors from Ad-BMP4-treated tumors and Ad-Fc-treated (control) tumors were isolated and hybridized to the microarrays according to the manufacturer's instructions. Scanned array background adjustment and signal intensity normalization were performed with GCRMA algorithm in the open-source bioconductor software (www.bioconductor.org). Genes differentially expressed between the two groups were identified with Bayesian t-test (Baldi and Long, Bioinformatics 17:509, 2001). The data were expressed in “fold-change” relative to control tumors. A gene was considered regulated when the treatment changed the expression level at least 2-fold with a P value less than or equal to 0.05.

The microarray analysis showed that 899 and 1136 human genes were up-regulated and down-regulated, respectively in the MIN-treated tumors. The regulation of several BMP target genes is evidence that the BMP signaling pathway is activated in response to BMP4 over-expression. The induction of well-known BMP pathway inhibitors such as Gremlin, Smad7 and Smurf1 may be evidence of an induction of a negative feed-back loop, further supporting the activity of adenovirus delivered BMP4. Additional major pathways were affected, for example, Nodal and Writ family members were mostly down-regulated, while several members of the TGE-β pathway were up-regulated. It addition, BMP4 induced the activation of the COX/PGE2 pathway, which has been associated with anti-tumor activity. The BMP4 treatment also impacted many genes involved in cell adhesion/mobility. Most adhesion molecules were significantly up-regulated along with some epithelial-mesenchymal transition (EMT) markers.

A selection of genes affected by BMP4 expression in OMP-C18 tumor cells is presented in Table 2 and is classified by biological pathway.

TABLE 2

| | | Expression | |
|---|---|---|---|
| Pathway | Gene Symbol | Regulation | Function |
| BMP | GREM1 | UP | BMP antagonist |
| | SMAD7 | UP | Intracellular BMP |
| | | | inhibitor |
| | | | Beta-catenin regulator |
| | SMURF1 | UP | Intracellular BMP |
| | | | inhibitor |
| | MSX2 | UP | BMP and Wnt target |
| | | | gene |
| Nodal | ACVR1C | DOWN | Receptor type 1 for |
| | | | Nodal |
| | TDGF-1 | DOWN | Co-receptor for Nodal |
| | | | BMP and Wnt target |
| | | | gene |
| TGF-beta | ACVRL1 | UP | TGF-beta receptor type 1 |
| | | | EC-specific |
| | | | Angiogenesis |
| | CDKN2B | UP | TGF-beta target gene |
| | | | Cell cycle arrest |
| | TGIF2 | DOWN | TGF-beta/Nodal/BMP |
| | | | inhibitor |
| | TIAF1 | UP | TGF-beta target gene |
| | | | Apoptosis |
| | DAB2 | UP | Signal transduction |
| | | | Wnt inhibitor |
| Wnt | DKK1 | UP | Wnt antagonist |
| | DVL2 | DOWN | Signal transduction |
| | FRAT2 | DOWN | Signal transduction |
| | APC | UP | Beta-catenin |
| | | | degradation |
| | ROR1 | DOWN | Non-canonical receptor |
| | LGR5 | DOWN | Wnt target gene |
| | DEFA5 | DOWN | Wnt target gene |
| | APCDD1 | DOWN | Wnt target gene |
| | EPHB2 | DOWN | Wnt target gene |
| | VANGL2 | DOWN | Wnt target gene |
| | ROBO1 | DOWN | Wnt target gene |
| COX2/PGE₂ | PTGDR | DOWN | PG receptor |
| | PTGER4 | DOWN | PG receptor |
| | PTGS2 | DOWN | PG receptor |
| Adhesion/ | CEACAM1, 6, 7 | UP | |
| mobility | and 8 | | |
| | ITGA6, B1, B4, | UP | |
| | B5 and B6 | | |
| | COL1A1, 17A1 | UP | EMT |
| | TWIST1 | UP | EMT |
| | VIM | UP | EMT |
| | MMP1, 14, 15 | UP | TGF-beta activation |
| | & 28 | | |
| Ligand/receptor | ADAM9, 10 and | UP | Ligand and/or receptor |
| processing | 19 | | processing |

### Example 7

### Generation of Anti-BMPR1A Monoclonal Antibodies

Antibodies were generated against an extracellular domain of human BMPR1A. Standard recombinant DNA technology was used to isolate polynucleotides encoding the extracellular domain of human BMPR1A (aa 1-152 of SEQ ID NO:1). The polynucleotide was ligated in-frame N-terminal to either a human Fc-tag, histidine-tag or FLAG-tag. The construct was cloned into a transfer plasmid vector for baculovirus-mediated expression in insect cells. Standard transfection, infection, and cell culture protocols were used to produce recombinant insect cells expressing the human BMPR1A polypeptide corresponding to the extracellular domain of human BMPR1A comprising amino acids 1-152 of SEQ ID NO:1) (O'Reilly et al., 1994, Baculovirus Expression Vectors: A Laboratory Manual, Oxford: Oxford University Press).

The extracellular domain of human BMPR1A polypeptide with His-tag was purified from insect cell supernatant using Protein A and Ni⁺⁺-chelate affinity chromatography as known to one skilled in the art. Purified human BMPR1A polypeptide was dialyzed against PBS (pH=7), concentrated to approximately 1 mg/ml, and sterile filtered in preparation for immunization.

Mice (n=3) were immunized with the purified human BMPR1A antigen protein described above using standard techniques. Blood from individual mice was screened approximately 70 days after initial immunization for antigen recognition using FACS analysis. The animal with the highest antibody titer was selected for final antigen boost after which spleen cells were isolated for hybridoma production. SP2/0 cells were used as fusion partners for the mouse spleen cells. Hybridoma cells were plated at 1 cell per well in 96 well plates, and the supernatant from each well screened by FACS analysis against human BMPR1A polypeptide. Several hybridomas with high antibody titer were selected and scaled up in static flask culture. Antibodies were purified from the hybridoma supernatant using protein A or protein G agarose chromatography. Purified monoclonal antibodies were assayed again by FACS and were isotyped to select for IgG antibodies.

Several antibodies that recognized the extracellular domain of human BMPR1A were isolated. A hydridoma cell line expressing antibody 5M107 was deposited under the conditions of the Budapest Treaty on Mar. 17, 2010 and assigned ATCC Patent Deposit Designation PTA-10720 The nucleotide and predicted protein sequences of the heavy chain variable region (SEQ ID NO:11 (nt) and SEQ ID NO:12 (aa)) of antibody 5M107 were determined.

### Example 8

### Effect of Anti-BMPR1A Antibody 5M107 on BMP4-Induced Gene Expression

The activity of anti-BMPR1A antibody 5M107 and BMPR1A-Fc was tested in a BMP4-induced differentiation assay. Mouse C2C12 cells were seeded into 24 well plates at 8×10⁴ cells per well in 0.5 ml medium (DMEM, 10% FBS, 5% NZ Cosmic calf serum). BMP4, a combination of BMP4 and human BMPR1A-Fc, or a combination of BMP4, BMPR1A-Fc and antibody 5M107 were tested in the assay. BMPR1A-Fc was pre-incubated for 30 minutes at 37° C. with a control antibody or 5M107, BMP4 was then added and incubated for an additional 30 minutes. The mixture was then added to the cells which were incubated for 24 hours at 37° C. and 5% CO₂. The final concentrations were: BMP4 at 200 ng/ml, BMPR1A-Fc at 5 ug/ml and 5M107 at 20 ug/ml. After incubation, RNA was extracted using a Qiagen RNeasy™ mini kit according to the manufacturer's protocol. 50 ng of RNA were analyzed by qPCR for Sp7 and GusB gene expression levels. The data were analyzed and Sp7 mRNA levels were calculated relative to GusB mRNA levels using SBI SDS2.2.1 software. The results (relative quantity) were compared to a BMP4 alone control sample.

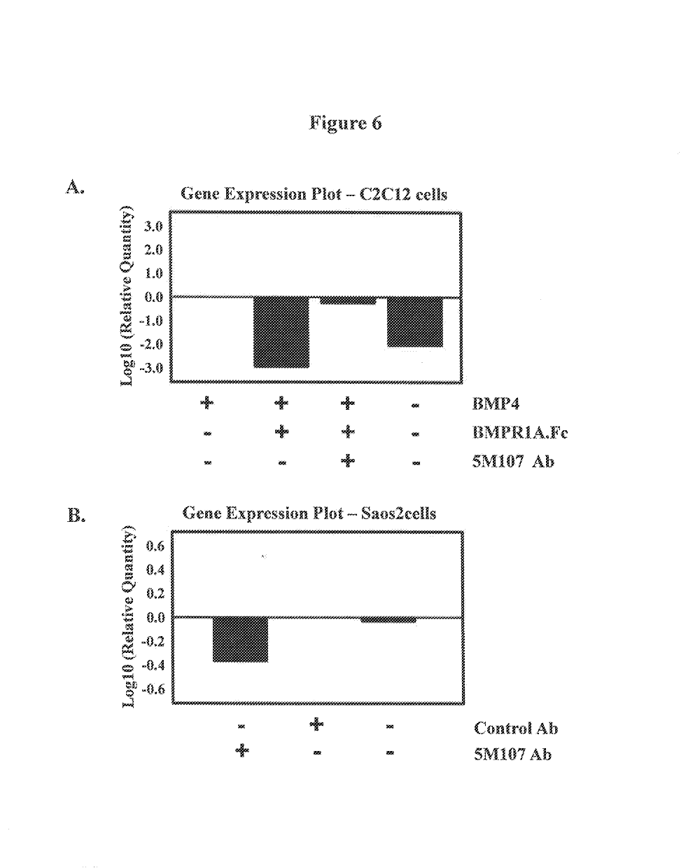

The BMPR1A-Fc fusion protein completely abrogated BMP4-induced Sp7 gene expression in C2C12 cells (FIG. 6A). These results translated into an inhibition of cell differentiation, most likely due to binding of the BMP4 to BMPR1A-Fc acting as a decoy receptor. This effect was completely reversed by the presence of anti-BMPR1A antibody 5M107. This experiment demonstrated binding of 5M107 to human BMPR1A (e.g. BMPR1A-Fc and/or cell surface BMPR1A) and that 5M107 competed with BMP4 for binding of BMPR1A.

The ability of anti-BMPR1A antibody 5M107 to block basal BMP signaling in a human system was evaluated in Saos2 cells. The cells were cultured in Macoy5A medium, 5% FBS and display detectable levels of Sp7 in the absence of BMP4. 8×10⁴ cells were seeded in 24-well plates and 20 μg/ml control antibody or anti-BMPR1A antibody 5M107 were added to the cells. After 24 hours, RNA was extracted using a Qiagen RNeasy™ mini kit according to the manufacturer's protocol. The data were analyzed and Sp7 mRNA levels were calculated relative to GusB mRNA levels using SBI SDS2.2.1 software. The results (relative quantity) were compared to a BMP4 alone control sample.

Anti-BMPR1A antibody 5M107 decreased Sp7 gene expression levels 2.4 fold relative to the control antibody (FIG. 6B). These results demonstrated that anti-BMPR1A antibody 5M107 inhibited endogenous BMP signaling in human cells.

### Example 9

### Anti-BMPR1A Antibody Treatment of OMP-C18 Tumors

Single cell suspensions of colon tumor OMP-C18 were obtained from minimally passaged xenografts by mechanical dissociation and enzymatic digestion with collagenase III and DNaseI for 2 hours at 37° C. Approximately 50,000 cells were injected subcutaneously in the flanks of NOD-SCID mice. On day 29 when the tumors reached an average size of 150 mm³, the mice were randomized, and placed in groups of 10. 15 mg/kg of anti-BMPR1A antibody 5M107 or control antibody LZ1 were injected intraperitoneally weekly. Tumor growth was monitored weekly and tumors were measured with a digital caliper. The experiment was terminated when the fastest growing tumor reached 1500 mm³ in size. Tumors were harvested and individually processed for FACS analysis, limiting dilution analysis and microarray gene expression profiling.

Single cell suspensions were obtained from the anti-BMPR1A antibody-treated and the control-treated tumors by mechanical dissociation and enzymatic digestion with collagenase III and DNaseI for 2 hours at 37° C. Approximately 1×10⁶ cells of each tumor were incubated in 100 μl of staining solution with a mixture of the following antibodies: 1 μl biotinylated anti-mouse H-2Kd, 0.5 μl biotinylated anti-mouse CD45, 2 μl allophycocyanin (APC)-conjugated anti-human ESA and 2 μl PE-Cy7-conjugated anti-human and anti-mouse CD44. A second incubation with 0.5 μl PE-Cy5.5-conjugated streptavidin was performed to detect the mouse cells bound with biotinylated antibodies. DAPI was added to the final solution to allow for detection of dead cells. The cells were analyzed on a CANTOII FACS instrument (BD Biosciences, San Jose, Calif.) and the data was processed using DIVA software.

For the limiting dilution assay (LDA), the human tumor cells in a cell suspension prepared as described above, were counted, and a series of cell doses (30, 90, and 270 cells) were injected subcutaneously in the flanks of a series of NOD/SCID mice (10 mice per cell dose per treatment group). Tumor take and tumor volume were assessed after 49 days.

For microarray analysis, total RNA was extracted from homogenized whole tumors using Qiagen's RNeasy® mini-prep kit. The global gene expression profiling analysis was performed on Affymetrix Human Genome U133 Plus 2.0 and Mouse Genome 430 2.0 array chips (Affymetrix, Santa Clara, Calif.). Three independent RNA samples of xenograft whole tumors from anti-BMPR1A antibody-treated tumors and control-treated tumors were isolated and hybridized to the microarrays according to the manufacturer's instructions. Scanned array background adjustment and signal intensity normalization were performed with GCRMA algorithm in the open-source bioconductor software (www.bioconductor.org). Genes differentially expressed between the two groups were identified with Bayesian t-test (Baldi and Long, Bioinformatics 17:509, 2001). The data were expressed in “fold-change” relative to control tumors. A gene was considered regulated when the treatment changed the expression level at least 2-fold with a P value less than or equal to 0.05.

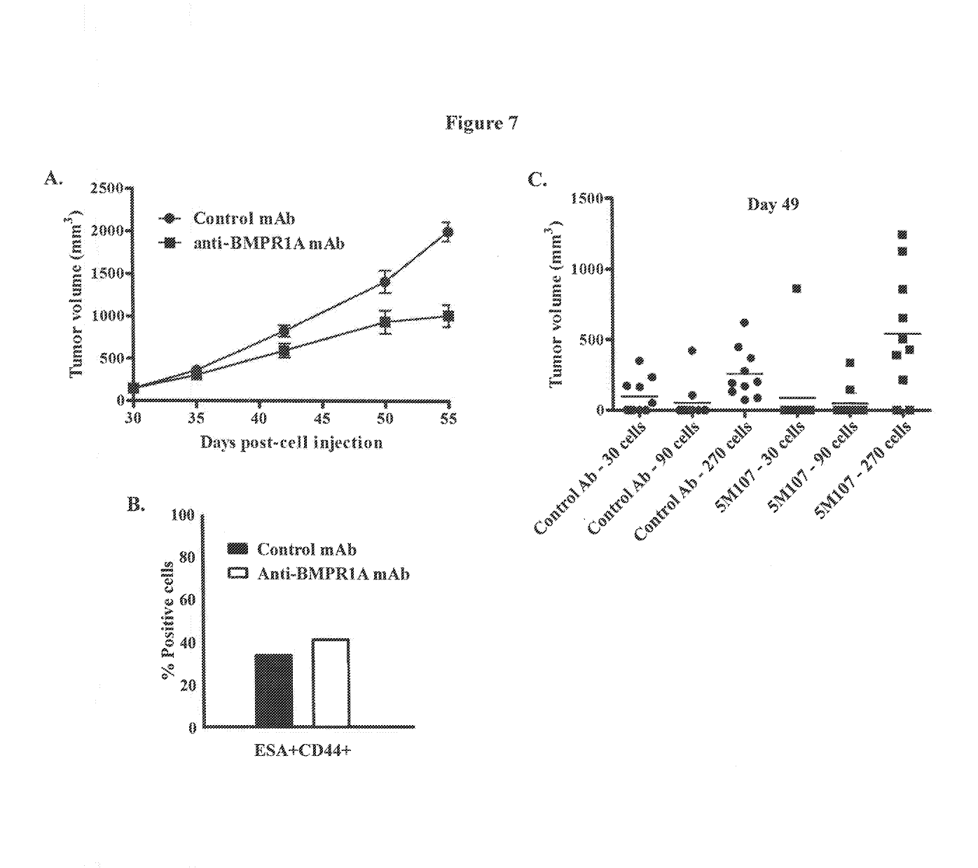

As shown in FIG. 7A, treatment with anti-BMPR1A antibody inhibited growth of the OMP-C18 tumor as compared to treatment with the control antibody. However, treatment with anti-BMPR1A antibody did not appear to have any effect on the percentage of ESA-expressing or CD44-expressing tumor cells (FIG. 7B). Treatment with anti-BMPR1A antibody 5M107 did not appear to reduce CSC frequency in this study (FIG. 7C). Thus, 5M107 inhibited tumor growth by inhibiting BMP pathway signaling and without reducing the frequency of CSC cells in a colon tumor xenograft model.

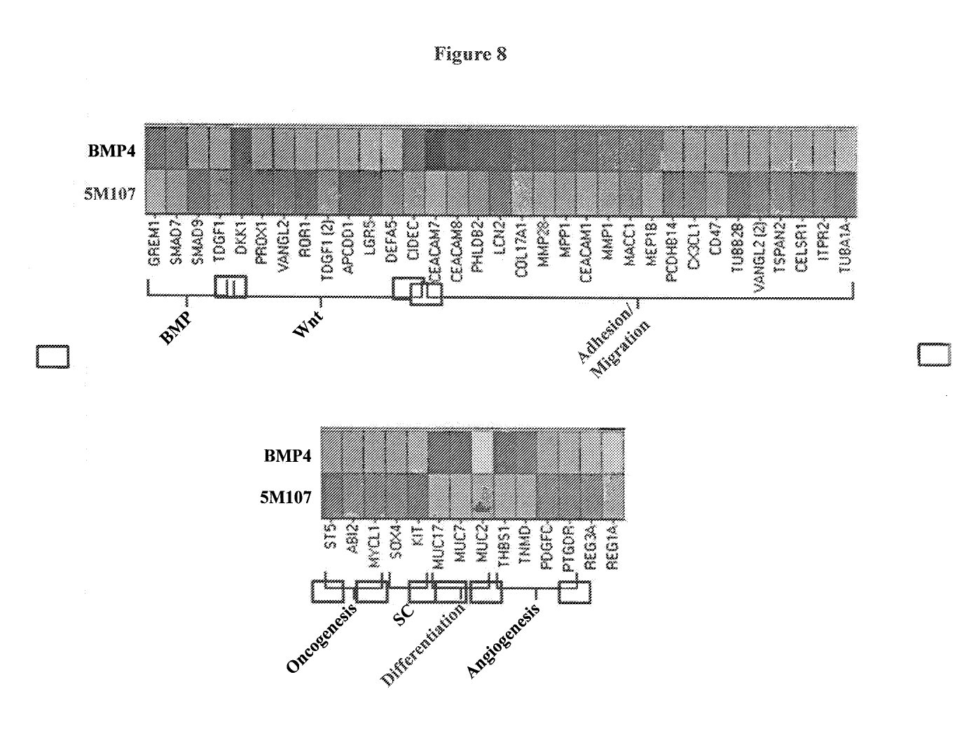

The microarray gene profile of the anti-BMPR1A antibody 5M107-treated tumors was compared to the microarray gene profile of OMP-C18 tumors over-expressing BMP4 (see Example 6). As shown in FIG. 8, the expression of BMP, Wnt, adhesion, oncogenesis, stem cell, differentiation and angiogenesis-related genes was regulated in an opposite fashion by the two treatments. These results indicate that over-expression of BMP4 and treatment with anti-BMPR1A antibody 5M107 inhibited tumor growth by different mechanisms.

### Example 10

### BMPR2 Expression Levels from Human Colon Tumors

Total RNA was extracted from primary human colon tumors OMP-C11, OMP-C17, OMP-C18, UM-C6 and OMP-C8. Gene expression profiles were established for each tumor type sample using Affymetrix microarray technology. The global gene expression profiling analysis was performed on Affymetrix Human Genome U133 Plus 2.0 and Mouse Genome 430 2.0 array chips (Affymetrix, Santa Clara, Calif.).

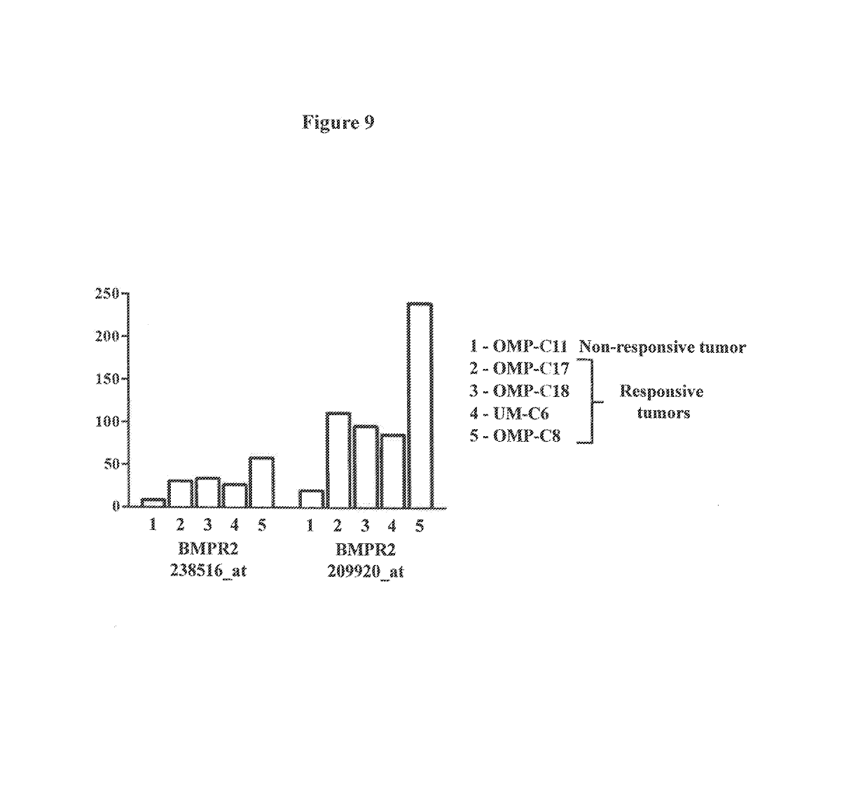

The data corresponding to two different BMPR2 probes were extracted and are shown in FIG. 9. Expression levels of BMPR2 were much lower in tumor OMP-C11 as compared to the other colon tumors. Interestingly, OMP-C11 was the only colon tumor studied that was non-responsive to treatment with BMP4 in an in vivo xenograft model (see Example 1), suggesting that BMPR2 may be involved in BMP4-induced tumor growth inhibition.

### Example 11

### Reporter Cell Lines

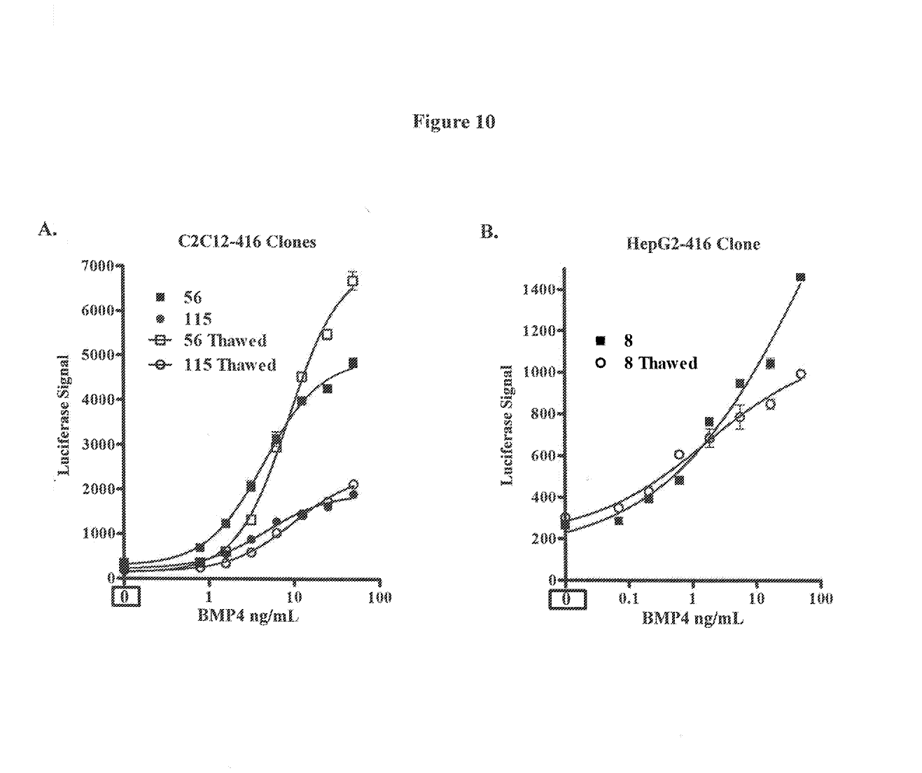

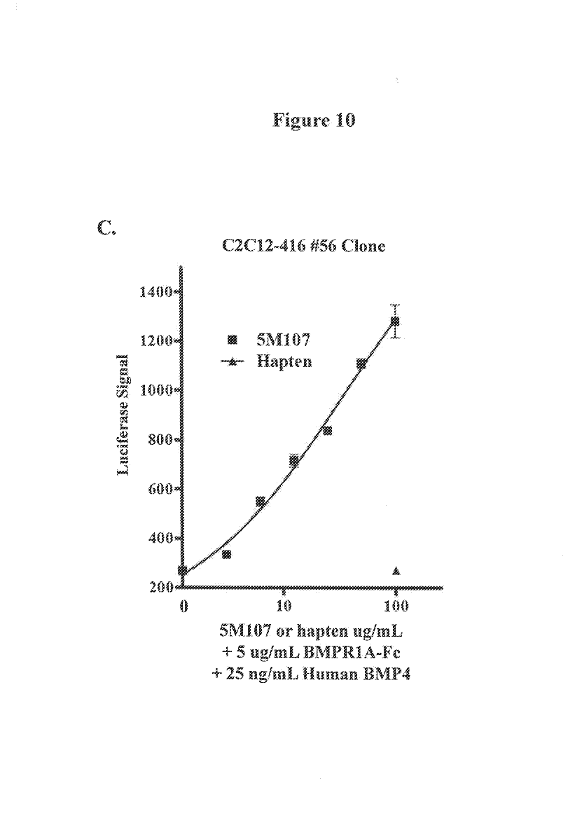

Two cell lines were established that express a luciferase reporter gene in response to BMP activation. A BRE-luciferase (BMP Responsive Element-Luciferase) cassette was assembled as described by Korchynskyi and Dijke, 2002, JBC, 277:4883-4891. The cassette was stably introduced into mouse C2C12 cells and human HepG2 cells using G418 as a selection marker. A clone was identified for each cell line that responded to BMP4 in a dose-dependent manner. Reporter cells (C2C12 and HepG2) were plated on day 0 and a concentration range of purified BMP4 (R&D Systems, Minneapolis, Minn.) was added to the wells. The cells were incubated overnight and assayed for luciferase expression using a Promega Steady Glow kit. As shown in FIG. 10, both C2C12 reporter cells (FIG. 10A) and HepG2 reporter cells (FIG. 10B) demonstrated a dose-dependent increase in luciferase expression in response to increasing concentrations of BMP4.

In an additional experiment, C2C12 reporter cells were plated on day 0, and 25 ng/ml human BMP4 and 5 μg/ml BMPR1A-Fc were added to the wells. BMPR1A-Fc is a decoy receptor which was capable of inhibiting the activation of the luciferase activity by BMP4 (FIG. 10C). Importantly, anti-BMPR1A antibody 5M107 was able to block the inhibition of BMPR1A-Fc in a dose-dependent manner demonstrating the specificity of the reporter system (FIG. 10C).

These cells are used to screen BMPR bispecific antibodies for agonist activity and their ability to stimulate the BMP pathway.

It is understood that the examples and embodiments described herein are for illustrative purposes only and that various modifications or changes in light thereof will be suggested to persons skilled in the art and are to be included with the spirit and purview of this application.

All publications, patents, patent applications, internet sites, and accession numbers/database sequences including both polynucleotide and polypeptide sequences cited herein are hereby incorporated by reference herein in their entirety for all purposes to the same extent as if each individual publication, patent, patent application, internet site, or accession number/database sequence were specifically and individually indicated to be so incorporated by reference.

SEQUENCES

Human EMPR1A (predicted signal sequence underlined) (SEQ ID NO: 1)
MPQLYIYIRLLGAYLFIISRVQGQNLDSMLHGTGMKSDSDQKKSENGVTLAPEDTLPFLK
CYCSGHCPDDAINNTCITNGHCFAIIEEDDQGETTLASGCMKYEGSDFQCKDSPKAQLRR
TIECCRTNLCNQYLQPTLPPVVIGPFFDGSIRWLVLLISMAVCIIAMIIFSSCFCYKHYC
KSISSRRRYNRDLEQDEAFIPVGESLKDLIDQSQSSGSGSGLPLLVQRTIAKQIQMVRQV
GKGRYGEVWMGKWRGEKVAVKVFFTTEEASWFRETEIYQTVLMRHENILGFIAADIKGTG
SWTQLYLITDYHENGSLYDFLKCATLDTRALLKLAYSAACGLCHLHTEIYGTQGKPAIAH
RDLKSKNILIKKNGSCCIADLGLAVKFNSDTNEVDVPLNTRVGTKRYMAPEVLDESLNKN
HFQPYIMADIYSFGLIIWEMARRCITGGIVEEYQLPYYNMVPSDPSYEDMREVVCVKRLR
PIVSNRWNSDECLRAVLKLMSECWAHNPASRLTALRIKKTLAKMVESQDVKI
Human BMPR2 (predicted signal sequence underlined) (SEQ ID NO: 2)
MTSSLQRPWRVPWLPWTILLVSTAAASQNQERLCAFKDPYQQDLGIGESRISHENGTILC
SKGSTCYGLWEKSKGDINLVKQGCWSHIGDPQECHYEECVVTTTPPSIQNGTYRFCCCST
DLCNVNFTENFPPPDTTPLSPPHSFNRDETIIIALASVSVLAVLIVALCFGYRMLTGDRK
QGLHSMNMMEAAASEPSLDLDNLKLLELIGRGRYGAVYKGSLDERPVAVKVFSFANRQNF
INEKNIYRVPLMEHDNIARFIVGDERVTADGRMEYLLVMEYYPNGSLCKYLSLHTSDWVS
SCRLAHSVTRGLAYLHTELPRGDHYKPAISHRDLNSRNVLVKNDGTCVISDFGLSMRLTG
NRLVRPGEEDNAAISEVGTIRYMAPEVLEGAVNLRDCESALKQVDMYALGLIYWEIFMRC
TDLFPGESVPEYQMAFQTEVGNHPTFEDMQVLVSREKQRPKEPEAWKENSLAVRSLKETI
EDCWDQDAEARLTAQCAEERMAELMMIWERNKSVSPTVNPMSTAMQNERNLSHNRRVPKI
GPYPDYSSSSYIEDSIHHTDSIVKNISSEHSMSSTPLTIGEKNRNSINYERQQAQARIPS
PETSVTSLSTNTTTTNTTGLTPSTGMTTISEMPYPDETNLHTTNVAQSIGPTPVCLQLTE
EDLETNKLDPKEVDKNLKESSDENLMEHSLKQFSGPDPLSSTSSSLLYPLIKLAVEATGQ
QDFTQTANGQACLIPDVLPTQIYPLPKQQNLPKRPTSLPLNTKNSTKEPRLKFGSKHKSN
LKQVETGVAKMNTINAAEPHVVTVTMNGVAGRNHSVNSHAATTQYANGTVLSGQTTNIVT
HRAQEMLQNQFIGEDTRLNINSSPDEHEPLLRREQQAGHDEGVLDRLVDRRERPLEGGRT
NSNNNNSNPCSEQDVLAQGVPSTAADPGPSKPRRAQRPNSLDLSATNVLDGSSIQIGEST
QDGKSGSGEKIKKRVKTPYSLKRWRPSTWVISTESLDCEVNNNGSNRAVHSKSSTAVYLA
EGGTATTMVSKDIGMNCL
Human BMPR1B (predicted signal sequence underlined) (SEQ ID NO: 3)
MLLRSAGKLNVGTKKEDGESTAPTPRPKVLRCKCHHHCPEDSVNNICSTDGYCFTMIEED
DSGLPVVTSGCLGLEGSDFQCRDTPIPHQRRSIECCTERNECNKDLHPTLPPLKNRDFVD
GPIHHRALLISVTVCSLLLVLIILFCYFRYKRQETRPRYSIGLEQDETYIPPGESLRDLI
EQSQSSGSGSGLPLLVQRTIAKQIQMVKQIGKGRYGEVWMGKWRGEKVAVKVFFTTEEAS
WFRETEIYQTVLMRHENILGFIAADIKGTGSWTQLYLITDYHENGSLYDYLKSTTLDAKS
MLKLAYSSVSGLCHLHTEIFSTQGKPAIAHRDLKSKNILVKKNGTCCIADLGLAVKFISD
TNEVDIPPNTRVGTKRYMPPEVLDESLNRNHFQSYIMADMYSFGLILWEVARRCVSGGIV
EEYQLPYHDLVPSDPSYEDMREIVCIKKLRPSFPNRWSSDECLRQMGKLMTECWAHNPAS
RLTALRVKKTLAKMSESQDIKL
Human ACVR2A (predicted signal sequence underlined) (SEQ ID NO: 4)
MGAAAKLAFAVFLISCSSGAILGRSETQECLRFNANWEKDRTNQTGVEPCYGDKDKRRHC
FATWKNISGSIEIVKQGCWLDDINCYDRTDCVEKKDSPEVYFCCCEGNMCNEKFSYFPEM
EVTQPTSNPVTPKPPYYNILLYSLVPLMLIAGIVICAFWVYRHHKMAYPPVLVPTQDPGP
PPPSPLLGLKPLQLLEVKARGRFGCVWKAQLLNEYVAVKIFPIQDKQSWQNEYEVYSLPG
MKHENILQFIGAEKRGTSVDVDLWLITAFHEKGSLSDFLKANVVSWNELCHIAETMARGL
AYLHEDIPGLKDGHKPAISHRDIKSKNVLLKNNLTACIADFGLALKFEAGKSAGLTHGQV
GTRRYMAPEVLEGAINFQRDAFLRIDMYAMGLVLWELASRCTAADGPVDEYMLPFEEEIG
QHPSLEDMQEVVVHKKKRPVLRDYWQKHAGMAMLCETIEECWDHDAEARLSAGCVGERIT
QMQRLTNIITTEDIVTVVTMVTNVDFPPKESSL
Human ACVR2B (predicted signal sequence underlined) (SEQ ID NO: 5)
MTAPWVALALLWGSLCAGSGRGEAETRECIYYNANWELERTNQSGLERCEGEQDKRLHCY
ASWRNSSGTIELVKKGCWLDDFNCYDRQECVATEENPQVYFCCCEGNFCNERFTHLPEAG
GPEVTYEPPPTAPTLLTVLAYSLLPIGGLSLIVLLAFWMYRHRKPPYGHVDIHEDPGPPP
PSPLVGLKPLQLLEIKARGRFGCVWKAQLMNDFVAVKIFPLQDKQSWQSEREIFSTPGMK
HENLLQFIAAEKRGSNLEVELWLITAFHDKGSLTDYLKGNIITWNELCHVAETMSRGLSY
LHEDVPWCRGEGHKPSIAHRDFKSKNVLLKSDLTAVLADFGLAVRFEPGKPPGDTHGQVG
TRRYMAPEVLEGAINFQRDAFLRIDMYAMGLVLWELVSRCKAADGPVDEYMLPEEEEIGQ
HPSLEELQEVVVHKKMRPTIKDHWLKHPGLAQLCVTIEECWDHDAEARLSAGCVEERVSL
IRRSVNGTTSDCLVSLVTSVTNVDLPPKESSI
Mouse BMPR1A (predicted signal sequence underlined) (SEQ ID NO: 6)
MTQLYTYIRLLGACLFIISHVQGQNLDSMLHGTGMKSDLDQKKPENGVTLAPEDTLPFLK
CYCSGHCPDDAINNTCITNGHCFAIIEEDDQGETTLTSGCMKYEGSDFQCKDSPKAQLRR
TIECCRTNLCNQYLQPTLPPVVIGPFFDGSIRWLVVLISMAVCIVAMIIFSSCFCYKHYC
KSISSRGRYNRDLEQDEAFIPVGESLKDLIDQSQSSGSGSGLPLLVQRTIAKQIQMVRQV
GKGRYGEVWMGKWRGEKVAVKVFFTTEEASWFRETEIYQTVLMRHENILGFIAADIKGTG
SWTQLYLITDYHENGSLYDFLKCATLDTRALLKLAYSAACGLCHLHTEIYGTQGKPAIAH
RDLKSKNILIKKNGSCCIADLGLAVKFNSDTNEVDIPLNTRVGTKRYMAPEVLDESLNKN
HFQPYIMADIYSFGLIIWEMARRCITGGIVEEYQLPYYNMVPSDPSYEDMREVVCVKRLR
PIVSNRWNSDECLRAVLKLMSECWAHNPASRLTALRIKKTLAKMVESQDVKI
Mouse BMPR1B (predicted signal sequence underlined) (SEQ ID NO: 7)
MLLRSSGKLNVGTKKEDGESTAPTPRPKILRCKCHHHCPEDSVNNICSTDGYCFTMIEED
DSGMPVVTSGCLGLEGSDFQCRDTPIPHQRRSIECCTERNECNKDLHPTLPPLKDRDFVD
GPIHHKALLISVTVCSLLLVLIILFCYFRYKRQEARPRYSIGLEQDETYIPPGESLRDLI
EQSQSSGSGSGLPLLVQRTIAKQIQMVKQIGKGRYGEVWMGKWRGEKVAVKVFFTTEEAS
WFRETEIYQTVLMRHENILGFIAADIKGTGSWTQLYLITDYHENGSLYDYLKSTTLDAKS
MLKLAYSSVSGLCHLHTEIFSTQGKPAIAHRDLKSKNILVKKNGTCCIADLGLAVKFISD
TNEVDIPPNTRVGTKRYMPPEVLDESLNRNHFQSYIMADMYSFGLILWEIARRCVSGGIV
EEYQLPYHDLVPSDPSYEDMREIVCMKKLRPSFPNRWSSDECLRQMGKLMTECWAQNPAS
RLTALRVKKTLAKMSESQDIKL
Mouse ACVR2A (predicted signal sequence underlined) (SEQ ID NO: 8)
MGAAAKLAFAVFLISCSSGAILGRSETQECLFFNANWERDRTNQTGVEPCYGDKDKRRHC
FATWKNISGSIEIVKQGCWLDDINCYDRTDCIEKKDSPEVYFCCCEGNMCNEKFSYFPEM
EVTQPTSNPVTPKPPYYNILLYSLVPLMLIAGIVICAFWVYRHHKMAYPPVLVPTQDPGP
PPPSPLLGLKPLQLLEVKARGRFGCVWKAQLLNEYVAVKIFPIQDKQSWQNEYEVYSLPG
MKHENILQFIGAEKRGTSVDVDLWLITAFHEKGSLSDFLKANVVSWNELCHIAETMARGL
AYLHEDIPGLKDGHKPAISHRDIKSKNVLLKNNLTACIADFGLALKFEAGKSAGDTHGQV
GTRRYMAPEVLEGAINFQRDAFLRIDMYAMGLVLWELASRCTAADGPVDEYMLPFEEEIG
QHPSLEDMQEVVVHKKKRPVLRDYWQKHAGMAMLCETIEECWDHDAEARLSAGCVGERIT
QMQRLTNIITTEDIVTVVTMVTNVDFPPKESSL
Mouse ACVR2B (predicted signal sequence underlined) (SEQ ID NO: 9)
MTAPWAALALLWGSLCAGSGRGEAETRECIYYNANWELERTNQSGLERCEGEQDKRLHCY
ASWRNSSGTIELVKKGCWLDDFNCYDRQECVATEENPQVYFCCCEGNFCNERFTHLPEPG
GPEVTYEPPPTAPTLLTVLAYSLLPIGGLSLIVLLAFWMYRHRKPPYGHVDIHEDPGPPP
PSPLVGLKPLQLLEIKARGRFGCVWKAQLMNDFVAVKIFPLQDKQSWQSEREIFSTPGMK
HENLLQFIAAEKRGSNLEVELWLITAFHDKGSLTDYLKGNIITWNELCHVAETMSRGLSY
LHEDVPWCRGEGHKPSIAHRDFKSKNVLLKSDLTAVLADFGLAVRFEPGKPPGDTHGQVG
TRRYMAPEVLEGAINFQRDAFLRIDMYAMGLVLWELVSRCKAADGPVDEYMLPFEEEIGQ
HPSLEELQEVVVHKKMRPTIKDHWLKHPGLAQLCVTIEECWDHDAEARLSAGCVEERVSL
IRRSVNGTTSDCLVSLVTSVTNVDLLPKESSI
Mouse BMPR2 (predicted signal sequence underlined) (SEQ ID NO: 10)
MTSSLHRPFRVPWLLWAVLLVSTTAASQNQERLCAFKDPYQQDLGIGESRISHENGTILC
SKGSTCYGLWEKSKGDINLVKQGCWSHIGDPQECHYEECVVTTTPPSIQNGTYRFCCCST
DLCNVNFTENFPPPDTTPLSPPHSFNRDETIIIALASVSVLAVLIVALCFGYRMLTGDRK
QGLHSMNMMEAAAAEPSLDLDNLKLLELIGRGRYGAVYKGSLDERPVAVKVFSFANRQNF
INEKNIYRVPLMEHDNIARFIVGDERLTADGRMEYLLVMEYYPNGSLCKYLSLHTSDWVS
SCRLAHSVTRGLAYLHTELPRGDHYKPAISHRDLNSRNVLVKNDGACVISDFGLSMRLTG
NRLVRPGEEDNAAISEVGTIRYMAPEVLEGAVNLRDCESALKQVDMYALGLIYWEVFMRC
TDLFPGESVPDYQMAFQTEVGNHPTFEDMQVLVSREKQRPKFPEAWKENSLAVRSLKETI
EDCWDQDAEARLTAQCAEERMAELMMIWERNKSVSPTVNPMSTAMQNERNLSHNRRVPKI
GPYPDYSSSSYIEDSIHHTDSIVKNISSEHSMSSTPLTIGEKNRNSINYERQQAQARIPS
PETSVTSLSTNTTTTNTTGLTPSTGMTTISEMPYPDETHLHATNVAQSIGPTPVCLQLTE
EDLETNKLDPKEVDKNLKESSDENLMEHSLKQFSGPDPLSSTSSSLLYPLIKLAVEVTGQ
QDFTQAANGQACLIPDVPPAQIYPLPKQQNLPKRPTSLPLNTKNSTKEPRLKFGNKHKSN
LKQVETGVAKMNTINAAEPHVVTVTMNGVAGRSHNVNSHAATTQYANGAVPAGQAANIVA
HRSQEMLQNQFIGEDTRLNINSSPDEHEPLLRREQQAGHDEGVLDRLVDRRERPLEGGRT
NSNNNNSNPCSEQDILTQGVTSTAADPGPSKPRRAQRPNSLDLSATNILDGSSIQIGEST
QDGKSGSGEKIKRRVKTPYSLKRWRPSTWVISTEPLDCEVNNNGSDRAVHSKSSTAVYLA
EGGTATTTVSKDIGMNCL
5M107 Heavy chain variable region (predicted signal sequence underlined) (SEQ ID NO: 11)
ATGGAATGGAGCTGGATCTTTCTCTTTCTCCTGTCAGGAACTGCAGGTGTCCTCTCTGAG
GTCCAGCTGCAACAGTCTGGACCTGAGCTGGTGAAGCCTGGGACTTCAGTGAAGATATCC
TGCAAGGCTTCTGGTTACTCATTCACTGGCTACTACATGCACTGGGTGAAACAAAGTCAG
GTAAAGAGCCTTGAGTGGATTGGACGTATTAATCCTGACAATGGTGGTCGTACTTACAAC
CAGATTTTCAAGGACAAGGCCAGCTTGACTGTCCATAAGTCCTCCAGCACAGCCTACATG
GAGCTCCACAGCCTGACATCTGACGACTCTGCAGTCTATTATTGTACAAGAGAGAGGGGC
CAATATGGTAACTACGGGGGATTTTCTGACTGGGGCCAAGGGACTCTGGTCACT
5M107 Heavy chain variable region (predicted signal sequence underlined) (SEQ ID NO: 12)
MEWSWIFLFLLSGTAGVLSEVQLQQSGPELVKPGTSVKISCKASGYSFTGYYMHWVKQSQ
VKSLEWIGRINPDNGGRTYNQIFKDKASLTVHKSSSTAYMELHSLTSDDSAVYYCTRERG
QYGNYGGFSDWGQGTLVT
SM107 Heavy chain variable region without predicted signal sequence (SEQ ID NO: 13)
EVQLQQSGPELVKPGTSVKISCKASGYSFTGYYMHWVKQSQVKSLEWIGRINPDNGGRTY
NQIFKDKASLTVHKSSSTAYMELHSLTSDDSAVYYCTRERGQYGNYGGFSDWGQGTLVT
5M107 Heavy chain CDR1 (SEQ ID NO: 14)
TGYYMH
5M107 Heavy chain CDR2 (SEQ ID NO: 15)
RINPDNGGRTYNQIFKDK
5MI07 Heavy chain CDR3 (SEQ ID NO: 16)
RERGQYGNYGGFSD
FLAG Tag (SEQ ID NO: 17)
DYKDDDK

1-106. (canceled)

107. An isolated monoclonal antibody that specifically binds an extracellular domain of at least one bone morphogenetic protein receptor (BMPR) selected from the group consisting of: BMPR1A, BMPR1B, BMPR2, ACVR2A and ACVR2B, wherein the antibody is:
(i) an agonist of the BMP pathway, or
(ii) an antagonist of the BMP pathway.

108. The antibody of claim 107, which is a recombinant antibody, a monoclonal antibody, a chimeric antibody, a bispecific antibody, a humanized antibody, a human antibody, or an antibody fragment.

109. The antibody of claim 107, which:
(i) modulates BMP pathway activity;
(ii) stimulates BMP pathway activity,
(iii) increases BMPR activation, and/or
(iv) inhibits tumor growth.

110. A pharmaceutical composition comprising the antibody of claim 107 and a pharmaceutically acceptable carrier.

111. A cell comprising or producing the antibody of claim 107.

112. An isolated polynucleotide molecule comprising a polynucleotide that encodes the antibody of claim 107.

113. A method of modulating BMP pathway signaling in a cell, comprising contacting the cell with an effective amount of the antibody of claim 107.

114. The method of claim 113, wherein the modulation is a stimulation of BMP pathway signaling.

115. A method of inhibiting tumor growth in a subject, comprising administering to the subject a therapeutically effective amount of the antibody of claim 107.

116. The method of claim 115, wherein the tumor is a colorectal tumor, a breast tumor, a prostate tumor, a pancreatic tumor, a lung tumor, a head and neck tumor, a glioblastoma tumor or a melanoma tumor.

117. The method of claim 115, further comprising administering to the subject at least one additional therapeutic agent.

118. A method of treating cancer in a subject, comprising administering to the subject a therapeutically effective amount of the antibody of claim 107.

119. The method of claim 118, wherein the cancer is colorectal cancer, breast cancer, prostate cancer, pancreatic cancer, lung cancer, glioblastoma, head and neck cancer or melanoma.

120. A bispecific antibody that specifically binds an extracellular domain of at least one BMPR selected from the group consisting of: BMPR1A, BMPR1B, BMPR2, ACVR2A and ACVR2B, wherein the antibody is an agonist of the BMP pathway.

121. The bispecific antibody of claim 120, which binds a type I BMPR and a type II BMPR.

122. The bispecific antibody of claim 121, wherein the type I BMPR is selected from the group consisting of: BMPR1A and BMPR1B, and the type II BMPR is selected from the group consisting of: BMPR2, ACVR2A and ACVR2B.

123. An isolated antibody that specifically binds an extracellular domain of BMPR1A, wherein the antibody comprises: a heavy chain CDR1 comprising TGYYMK (SEQ ID NO:14), a heavy chain CDR2 comprising RINPDNGGRTYNQIFKDK (SEQ ID NO:15), and a heavy chain CDR3 comprising RERGQYGNYGGFSD (SEQ ID NO:16).

124. The antibody of claim 123, which comprises a heavy chain variable region having at least about 90% sequence identity to SEQ ID NO:13.

125. A monoclonal antibody produced by the hybridoma cell line 5M107 on deposit as ATCC Patent Deposit no. PTA-10720.

126. A humanized form of the antibody of claim 125.


SEQUENCE LISTING (17 sequences)

SEQ ID NO: 1 - protein, 532 aa, Homo sapiens
  BMPR1A
MPQLYIYIRLLGAYLFIISRVQGQNLDSMLHGTGMKSDSDQKKSENGVTLAPEDTLPFLKCYCSGHCPDDAINNTCITNGHCFAIIEEDDQGETTLASGCMKYEGSDFQCKDSPKAQLRRTIECCRTNLCNQYLQPTLPPVVIGPFFDGSIRWLVLLISMAVCIIAMIIFSSCFCYKHYCKSISSRRRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDVPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 2 - protein, 1038 aa, Homo sapiens
  BMPR2
MTSSLQRPWRVPWLPWTILLVSTAAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAASEPSLDLDNLKLLELIGRGRYGAVYKGSLDERPVAVKVFSFANRQNFINEKNIYRVPLMEHDNIARFIVGDERVTADGRMEYLLVMEYYPNGSLCKYLSLHTSDWVSSCRLAHSVTRGLAYLHTELPRGDHYKPAISHRDLNSRNVLVKNDGTCVISDFGLSMRLTGNRLVRPGEEDNAAISEVGTIRYMAPEVLEGAVNLRDCESALKQVDMYALGLIYWEIFMRCTDLFPGESVPEYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETNLHTTNVAQSIGPTPVCLQLTEEDLETNKLDPKEVDKNLKESSDENLMEHSLKQFSGPDPLSSTSSSLLYPLIKLAVEATGQQDFTQTANGQACLIPDVLPTQIYPLPKQQNLPKRPTSLPLNTKNSTKEPRLKFGSKHKSNLKQVETGVAKMNTINAAEPHVVTVTMNGVAGRNHSVNSHAATTQYANGTVLSGQTTNIVTHRAQEMLQNQFIGEDTRLNINSSPDEHEPLLRREQQAGHDEGVLDRLVDRRERPLEGGRTNSNNNNSNPCSEQDVLAQGVPSTAADPGPSKPRRAQRPNSLDLSATNVLDGSSIQIGESTQDGKSGSGEKIKKRVKTPYSLKRWRPSTWVISTESLDCEVNNNGSNRAVHSKSSTAVYLAEGGTATTMVSKDIGMNCL

SEQ ID NO: 3 - protein, 502 aa, Homo sapiens
  BMPR1B
MLLRSAGKLNVGTKKEDGESTAPTPRPKVLRCKCHHHCPEDSVNNICSTDGYCFTMIEEDDSGLPVVTSGCLGLEGSDFQCRDTPIPHQRRSIECCTERNECNKDLHPTLPPLKNRDFVDGPIHHRALLISVTVCSLLLVLIILFCYFRYKRQETRPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEVARRCVSGGIVEEYQLPYHDLVPSDPSYEDMREIVCIKKLRPSFPNRWSSDECLRQMGKLMTECWAHNPASRLTALRVKKTLAKMSESQDIKL

SEQ ID NO: 4 - protein, 513 aa, Homo sapiens
  ACVR2A
MGAAAKLAFAVFLISCSSGAILGRSETQECLFFNANWEKDRTNQTGVEPCYGDKDKRRHCFATWKNISGSIEIVKQGCWLDDINCYDRTDCVEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 5 - protein, 512 aa, Homo sapiens
  ACVR2B
MTAPWVALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEAGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLPPKESSI

SEQ ID NO: 6 - protein, 532 aa, Mus musculus
  BMPR1A
MTQLYTYIRLLGACLFIISHVQGQNLDSMLHGTGMKSDLDQKKPENGVTLAPEDTLPFLKCYCSGHCPDDAINNTCITNGHCFAIIEEDDQGETTLTSGCMKYEGSDFQCKDSPKAQLRRTIECCRTNLCNQYLQPTLPPVVIGPFFDGSIRWLVVLISMAVCIVAMIIFSSCFCYKHYCKSISSRGRYNRDLEQDEAFIPVGESLKDLIDQSQSSGSGSGLPLLVQRTIAKQIQMVRQVGKGRYGEVWMGKWRGEKVAVKVFFTTEEASWFRETEIYQTVLMRHENILGFIAADIKGTGSWTQLYLITDYHENGSLYDFLKCATLDTRALLKLAYSAACGLCHLHTEIYGTQGKPAIAHRDLKSKNILIKKNGSCCIADLGLAVKFNSDTNEVDIPLNTRVGTKRYMAPEVLDESLNKNHFQPYIMADIYSFGLIIWEMARRCITGGIVEEYQLPYYNMVPSDPSYEDMREVVCVKRLRPIVSNRWNSDECLRAVLKLMSECWAHNPASRLTALRIKKTLAKMVESQDVKI

SEQ ID NO: 7 - protein, 502 aa, Mus musculus
  BMPR1B
MLLRSSGKLNVGTKKEDGESTAPTPRPKILRCKCHHHCPEDSVNNICSTDGYCFTMIEEDDSGMPVVTSGCLGLEGSDFQCRDTPIPHQRRSIECCTERNECNKDLHPTLPPLKDRDFVDGPIHHKALLISVTVCSLLLVLIILFCYFRYKRQEARPRYSIGLEQDETYIPPGESLRDLIEQSQSSGSGSGLPLLVQRTIAKQIQMVKQIGKGRYGEVWMGKWRGEKVAVKVFFTTEEASWFRETEIYQTVLMRHENILGFIAADIKGTGSWTQLYLITDYHENGSLYDYLKSTTLDAKSMLKLAYSSVSGLCHLHTEIFSTQGKPAIAHRDLKSKNILVKKNGTCCIADLGLAVKFISDTNEVDIPPNTRVGTKRYMPPEVLDESLNRNHFQSYIMADMYSFGLILWEIARRCVSGGIVEEYQLPYHDLVPSDPSYEDMREIVCMKKLRPSFPNRWSSDECLRQMGKLMTECWAQNPASRLTALRVKKTLAKMSESQDIKL

SEQ ID NO: 8 - protein, 513 aa, Mus musculus
  ACVR2A
MGAAAKLAFAVFLISCSSGAILGRSETQECLFFNANWERDRTNQTGVEPCYGDKDKRRHCFATWKNISGSIEIVKQGCWLDDINCYDRTDCIEKKDSPEVYFCCCEGNMCNEKFSYFPEMEVTQPTSNPVTPKPPYYNILLYSLVPLMLIAGIVICAFWVYRHHKMAYPPVLVPTQDPGPPPPSPLLGLKPLQLLEVKARGRFGCVWKAQLLNEYVAVKIFPIQDKQSWQNEYEVYSLPGMKHENILQFIGAEKRGTSVDVDLWLITAFHEKGSLSDFLKANVVSWNELCHIAETMARGLAYLHEDIPGLKDGHKPAISHRDIKSKNVLLKNNLTACIADFGLALKFEAGKSAGDTHGQVGTRRYMAPEVLEGAINFQRDAFLRIDMYAMGLVLWELASRCTAADGPVDEYMLPFEEEIGQHPSLEDMQEVVVHKKKRPVLRDYWQKHAGMAMLCETIEECWDHDAEARLSAGCVGERITQMQRLTNIITTEDIVTVVTMVTNVDFPPKESSL

SEQ ID NO: 9 - protein, 512 aa, Mus musculus
  ACVR2B
MTAPWAALALLWGSLCAGSGRGEAETRECIYYNANWELERTNQSGLERCEGEQDKRLHCYASWRNSSGTIELVKKGCWLDDFNCYDRQECVATEENPQVYFCCCEGNFCNERFTHLPEPGGPEVTYEPPPTAPTLLTVLAYSLLPIGGLSLIVLLAFWMYRHRKPPYGHVDIHEDPGPPPPSPLVGLKPLQLLEIKARGRFGCVWKAQLMNDFVAVKIFPLQDKQSWQSEREIFSTPGMKHENLLQFIAAEKRGSNLEVELWLITAFHDKGSLTDYLKGNIITWNELCHVAETMSRGLSYLHEDVPWCRGEGHKPSIAHRDFKSKNVLLKSDLTAVLADFGLAVRFEPGKPPGDTHGQVGTRRYMAPEVLEGAINFQRDAFLRIDMYAMGLVLWELVSRCKAADGPVDEYMLPFEEEIGQHPSLEELQEVVVHKKMRPTIKDHWLKHPGLAQLCVTIEECWDHDAEARLSAGCVEERVSLIRRSVNGTTSDCLVSLVTSVTNVDLLPKESSI

SEQ ID NO: 10 - protein, 1038 aa, Mus musculus
  BMPR2
MTSSLHRPFRVPWLLWAVLLVSTTAASQNQERLCAFKDPYQQDLGIGESRISHENGTILCSKGSTCYGLWEKSKGDINLVKQGCWSHIGDPQECHYEECVVTTTPPSIQNGTYRFCCCSTDLCNVNFTENFPPPDTTPLSPPHSFNRDETIIIALASVSVLAVLIVALCFGYRMLTGDRKQGLHSMNMMEAAAAEPSLDLDNLKLLELIGRGRYGAVYKGSLDERPVAVKVFSFANRQNFINEKNIYRVPLMEHDNIARFIVGDERLTADGRMEYLLVMEYYPNGSLCKYLSLHTSDWVSSCRLAHSVTRGLAYLHTELPRGDHYKPAISHRDLNSRNVLVKNDGACVISDFGLSMRLTGNRLVRPGEEDNAAISEVGTIRYMAPEVLEGAVNLRDCESALKQVDMYALGLIYWEVFMRCTDLFPGESVPDYQMAFQTEVGNHPTFEDMQVLVSREKQRPKFPEAWKENSLAVRSLKETIEDCWDQDAEARLTAQCAEERMAELMMIWERNKSVSPTVNPMSTAMQNERNLSHNRRVPKIGPYPDYSSSSYIEDSIHHTDSIVKNISSEHSMSSTPLTIGEKNRNSINYERQQAQARIPSPETSVTSLSTNTTTTNTTGLTPSTGMTTISEMPYPDETHLHATNVAQSIGPTPVCLQLTEEDLETNKLDPKEVDKNLKESSDENLMEHSLKQFSGPDPLSSTSSSLLYPLIKLAVEVTGQQDFTQAANGQACLIPDVPPAQIYPLPKQQNLPKRPTSLPLNTKNSTKEPRLKFGNKHKSNLKQVETGVAKMNTINAAEPHVVTVTMNGVAGRSHNVNSHAATTQYANGAVPAGQAANIVAHRSQEMLQNQFIGEDTRLNINSSPDEHEPLLRREQQAGHDEGVLDRLVDRRERPLEGGRTNSNNNNSNPCSEQDILTQGVTSTAADPGPSKPRRAQRPNSLDLSATNILDGSSIQIGESTQDGKSGSGEKIKRRVKTPYSLKRWRPSTWVISTEPLDCEVNNNGSDRAVHSKSSTAVYLAEGGTATTTVSKDIGMNCL

SEQ ID NO: 11 - DNA, 414 bp, Artificial sequence
  5M107 Heavy chain variable region
atggaatggagctggatctttctctttctcctgtcaggaactgcaggtgtcctctctgaggtccagctgcaacagtctggacctgagctggtgaagcctgggacttcagtgaagatatcctgcaaggcttctggttactcattcactggctactacatgcactgggtgaaacaaagtcaggtaaagagccttgagtggattggacgtattaatcctgacaatggtggtcgtacttacaaccagattttcaaggacaaggccagcttgactgtccataagtcctccagcacagcctacatggagctccacagcctgacatctgacgactctgcagtctattattgtacaagagagaggggccaatatggtaactacgggggattttctgactggggccaagggactctggtcact

SEQ ID NO: 12 - protein, 138 aa, Artificial sequence
  5M107 Heavy chain variable region
MEWSWIFLFLLSGTAGVLSEVQLQQSGPELVKPGTSVKISCKASGYSFTGYYMHWVKQSQVKSLEWIGRINPDNGGRTYNQIFKDKASLTVHKSSSTAYMELHSLTSDDSAVYYCTRERGQYGNYGGFSDWGQGTLVT

SEQ ID NO: 13 - protein, 119 aa, Artificial sequence
  5M107 Heavy chain variable region without predicted signal sequence
EVQLQQSGPELVKPGTSVKISCKASGYSFTGYYMHWVKQSQVKSLEWIGRINPDNGGRTYNQIFKDKASLTVHKSSSTAYMELHSLTSDDSAVYYCTRERGQYGNYGGFSDWGQGTLVT

SEQ ID NO: 14 - protein, 6 aa, Artificial sequence
  5M107 Heavy chain CDR1
TGYYMH

SEQ ID NO: 15 - protein, 18 aa, Artificial sequence
  5M107 Heavy chain CDR2
RINPDNGGRTYNQIFKDK

SEQ ID NO: 16 - protein, 14 aa, Artificial sequence
  5M107 Heavy chain CDR3
RERGQYGNYGGFSD

SEQ ID NO: 17 - protein, 7 aa, Artificial sequence
  FLAG Tag
DYKDDDK